(19) United States
(12) Patent Application Publication
(10) Pub. No.: US 2025/0270215 A1
(43) Pub. Date: Aug. 28, 2025

(54) IMIDAZOLE DERIVATIVES AS ALK5 INHIBITORS

(71) Applicant: CHIESI FARMACEUTICI S.P.A., Parma (IT)

(72) Inventors: Daniela PIZZIRANI, Parma (IT); Paolo RONCHI, Parma (IT); Matteo BIAGETTI, Parma (IT); Sara GUARIENTO, Parma (IT); Daniele PALA, Parma (IT)

(73) Assignee: CHIESI FARMACEUTICI S.P.A., Parma (IT)

(21) Appl. No.: 18/859,675

(22) PCT Filed: Apr. 26, 2023
(86) PCT No.: PCT/EP2023/060891

§ 371 Date: Oct. 24, 2024

(30) Foreign Application Priority Data
Apr. 27, 2022 (EP) 22170302.8

(51) Int. Cl. C07D 487/04; A61K 31/4188; A61K 31/4365; A61K 31/437; A61K 31/4439; A61K 31/444; C07D 519/00

(52) U.S. Cl. CPC C07D 487/04; A61K 31/4188; A61K 31/4365; A61K 31/437; A61K 31/4439; A61K 31/444; C07D 519/00

(57) ABSTRACT

The present invention relates to compounds inhibiting the transforming growth factor p (TGF f) type I receptor (ALK5) (hereinafter ALK5 inhibitors), methods of preparing such compounds, pharmaceutical compositions containing them and therapeutic use thereof. The compounds of the invention may be useful in the treatment of many diseases, disorders, or conditions associated with ALK5 signaling pathway.

### FIELD OF THE INVENTION

The present invention relates to compounds inhibiting the transforming growth factor B (TGF β) type I receptor (ALK5) (hereinafter ALK5 inhibitors), methods of preparing such compounds, pharmaceutical compositions containing them and therapeutic use thereof. The compounds of the invention may be useful in the treatment of many diseases, disorders, or conditions associated with ALK5 signaling pathway.

### BACKGROUND OF THE INVENTION

The Transforming Growth Factor β (TGF β) is a protein belonging to the TGF β superfamily.

It is involved in several processes, both cellular, such as proliferation, migration and differentiation, and biological, including wound healing, immunosuppression, cancerogenesis and extracellular matrix production.

The TGF β superfamily also includes, among others, other members known as activins (Acts) (see e.g. Hinck A P, FEBS Letters 586 (2012); 1860-1870).

The binding of the peptide initiates the TGF β signaling cascade through the formation of a heterotetrameric complex composed of two different serine/threonine kinases receptors: type 1 (TGFβR1/ALK5) and type 2 (TGFβR2).

TGFβR1/ALK5 is recruited and activated through the phosphorylation of its intracellular domain by TGFβR2, leading in turn to the phosphorylation of the receptor-activated (R)-Smad family, resulting in the activation of target gene transcription (see e.g. Sheppard D., Proc Am Thorac Soc. (2006); (3): 413-417).

Similarily to the TGF β signaling, the type I receptor for activin, ALK4, leads to the activation of target gene transcription (see e.g. Heldin C H et al., Cold Spring Harb Perspect Biol. (2016) August 1; 8 (8)).

Several studies have linked an excessive and/or dysregulated TGFβ activity with many diseases including cancer and fibrosis (see e.g. Syed V, J Cell Biochem. (2016) June; 117 (6): 1279-87; Jakowlew S B. Cancer Metastasis Rev. (2006) September; 25 (3): 435-57). Among fibrotic disorders, a crucial role of TGFβ has been shown in organs such as lung, heart, liver, and kidney (see e.g. Alhamad E H, J Thorac Dis. (2015); 7 (3): 386-93). In particular, TGFβ expression is increased in fibrotic lung diseases, such as idiopathic pulmonary fibrosis (IPF), and in chronic inflammatory conditions, such as chronic obstructive pulmonary disease and asthma (see e.g. Thomas B J et al., Am J Respir Cell Mol Biol. (2016); (55): 759-766).

In lung, TGFB is expressed in several cell types, like epithelial cells, endothelial cells, connective tissue cells, macrophages and fibroblasts.

These cell populations may produce excess of TGFβ in IPF human lung tissue. Moreover, high levels of TGFB have been detected in lung tissue and BAL of IPF patients (see e.g., Bergeron A et al., Eur Respir J (2003); 22:69-76).

TGFβ gene expression and TGFβ protein production have been observed to increase in a variety of animal models of pulmonary fibrosis caused by bleomycin, silica, asbestos, and radiation (see e.g., Wei F et al., Int Immunopharmacol. (2017) July; 48:67-75; Choe J Y et al., Inflamm Res. (2010) March; 59 (3): 177-88; Wang X et al., Respir Res (2009); 10, 36) and it has also been reported how the TGFB expression is sufficient to induce progressive fibrosis in rodents (see e.g., Sime P J et al., J Clin Invest (1997); 100:768-776; Kim K K et al.).

Contrarily, TGFβ signaling inhibition obtained by employing knockout (KO) animals can inhibit fibrosis development through TGFβ-linked mechanisms (see e.g., Bonniaud P et al., Am J Respir Crit Care Med (2005); 171:889-898; 34).

Similar results have been achieved with inhibition of TGFβR1 in mouse bleomycin disease model (see e.g., Wei Y et al., J Clin Invest. (2017); 127 (10): 3675-3688).

Activin signalling dysregulation, similarly to TGFB, is associated to fibroblasts proliferation, myofibroblasts differentiation and accumulation of extracellular matrix (ECM) (see e.g. Yamashita et al., J. Am. Soc. Nephrol. (2004) 15, 91-101). Moreover, overexpression of activin has been linked to pathological conditions and fibrosis development in different organs, such as liver (see e.g., Patella et al., Am. J. Physiol. Gastrointest. Liver Physiol. (2006) 290, G137-G144), kidney (see e.g., Agapova et al., Kidney Int. (2016) 89, 1231-1243), heart (see e.g. Yndestad et al., Circulation (2004) 109, 1379-1385), and lung (see e.g. de Kretser et al., Crit. Care (2013) 17:R263).

Taken together these data suggest the importance of targeting ALK5 receptor to treat pharmacologically the aforementioned diseases linked to dysregulated TGF signaling pathway.

The TGFβ signaling is strongly involved in the cardiovascular homeostasis (see e.g., van Meeteren L A et al, Springer (2013)) Several studies in humans and mice have shown the main role of TGFβ in angiogenesis and vascular morphogenesis. Moreover, TGFβ plays a key role in the development and functionality of cardiac valves. It is therefore clear the importance of a selective regulation of TGFB pathway to target the pathological effects avoiding the suppression of the signaling needed for a correct homeostasis.

The answer to this crucial point could be addressed by using the inhalation route to deliver an antiTGFβ drug.

The inhalatory route would allow the treatment of the affected lung compartment bypassing the issue of the heart exposure.

Various compounds have been described in the literature as ALK5 and/or ALK4 inhibitors.

WO2008/006583, WO2009/087212, WO2009/087224, WO2009/087225, WO2009/133070, WO2009/013335 and WO2009/050183 (Novartis) disclose respectively pyrimidine, pyridine, imidazo pyridine, pyrrolo pyrimidine and pyrrolo pyridine, imidazo pyridazine, imidazo pyridine derivatives for the treatment of ALK4 or ALK5 mediated diseases useful for the treatment of inflammatory or obstructive airways diseases, pulmonary hypertension and pulmonary fibrosis.

WO00/61576 and US2003/0149277 (Smithkline Beecham Corp) disclose triarylimidazole derivatives as ALK5 inhibitors useful for the treatment of, among others, renal disease, wound healing, kidney disease, congestive heart failure, ulcers, impaired neurological function and any disease wherein fibrosis is a major component.

WO01/62756 (Smithkline Beecham P.L.C.) discloses pyridinylimidazole derivatives as ALK5 inhibitors useful for the treatment of, among others, renal disease, wound healing, kidney disease, congestive heart failure, ulcers, impaired neurological function and any disease wherein fibrosis is a major component.

WO03/087304 (Biogen Inc.) discloses tri-substituted heteroaryls as ALK5 and/or ALK4 inhibitors useful for the treatment of, among others, idiopathic pulmonary fibrosis, diabetic nephropathy, hepatic fibrosis, pulmonary fibrosis, acute lung injury, post-infarction cardiac fibrosis, fibrotic cancers and fibroma.

Imidazole derivatives have been disclosed in the literature as TGF-β inhibitors.

WO2013/009140 (SK Chemicals Co) discloses 2-pyridyl substituted imidazole derivatives as ALK5 and/or ALK4 receptors useful for the treatment of, among others, renal-, liver- or pulmonary fibrosis.

WO2016/081364 (Rigel Pharmaceuticals Inc.) discloses imidazole derivatives as TGF-β inhibitors useful for the treatment of fibrotic disorders, such as involved in chronic renal disease and vascular disease.

WO2020/041562 (Clavius Pharmaceuticals LLC) discloses imidazole derivatives as TGF-β inhibitors, useful for the treatment of, among others, multiple sclerosis, idiopathic pulmonary fibrosis, Alzheimer's Disease and chronic kidney disease.

Of note, inhibition of ALK5 receptor may be useful for the treatment of fibrosis and diseases, disorders and conditions that result from fibrosis.

Several efforts have been done in the past years to develop novel ALK5 receptor inhibitors useful for the treatment of several diseases and some of those compounds have shown efficacy also in humans.

However, there remains a potential for developing inhibitors of receptors ALK5 characterized by good potency, useful for the treatment of diseases or conditions associated with a dysregulation of ALK5 signaling pathway, in particular fibrosis.

In particular, there remains a potential for developing inhibitors of receptor ALK5 useful for the treatment of diseases or conditions associated with a dysregulation of ALK5 signaling in the respiratory field, in particular idiopathic pulmonary fibrosis (IPF), to be administered by the inhalation route and characterized by a good inhalatory profile, that corresponds to a good activity in the lung, a good lung retention and to a low metabolic stability in order to minimize the systemic exposure and correlated safety issues.

In this direction, we have surprisingly found a new series of compounds of general formula (I) that solves the problem of providing potent inhibitors of ALK5 receptor for administration by inhalation, that shows, at the same time, a good inhalatory profile, low metabolic stability, low systemic exposure, improved safety and tolerability.

### SUMMARY OF THE INVENTION

In a first aspect the present invention relates to compounds of formula (I)

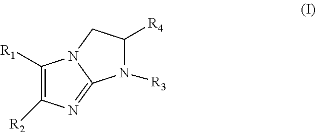

wherein
R₁ is selected from the group consisting of aryl optionally substituted by one or more groups selected from halogen atoms, NH₂, —OH, —O(C₁-C₆)alkyl, —S—(C₁-C₆)alkyl, —S(O)(C₁-C₆)alkyl, —S(O)₂—(C₁-C₆)alkyl, —S(O)₂—NH₂, —C(O)OH and —C(O)O—(C₁-C₆)alkyl; heterocycloalkyl optionally substituted by a oxo group; pyridyl substituted by NH₂; phenyl, pyridyl or thienyl fused with a structural moiety, which together with two ring members of said phenyl, pyridyl or thienyl, forms a 5-7 membered aromatic or non-aromatic ring, wherein said ring optionally contains up to three heteroatoms selected from N, O and S;
R₂ is aryl optionally substituted by one or more groups selected from halogen atoms and —(C₁-C₆)alkyl; or R₂ is heteroaryl optionally substituted by one or more groups selected from —(C₁-C₆)alkyl, —O—(C₁-C₆)alkyl, —OH and halogen atoms;
R₃ is H or is independently selected from the group consisting of —(C₁-C₆)alkyl and —C(O)—(C₁-C₆)alkyl;
R₄ is H or is selected from the group consisting of —(C₁-C₆)hydroxyalkyl, —C(O)OR₅, —C(O)O—(C₁-C₆)alkyl, —C(O)—NR₅R₆, —(C₁-C₆)alkylene-C(O)OH and —(C₁-C₆)alkylene-C(O)O—(C₁-C₆)alkyl;
R₅ is H or —(C₁-C₆)alkyl;
R₆ is —(C₁-C₆)alkyl;
and pharmaceutically acceptable salts thereof.

In a second aspect, the invention refers to a pharmaceutical composition comprising a compound of formula (I) and pharmaceutically acceptable salts thereof in admixture with one or more pharmaceutically acceptable carrier or excipient.

In a third aspect, the invention refers to a compound of formula (I) and pharmaceutically acceptable salts or to a pharmaceutical composition comprising a compound of formula (I) and pharmaceutically acceptable salts thereof for use as a medicament.

In a further aspect, the invention refers to a compound of formula (I) and pharmaceutically acceptable salts thereof or to a pharmaceutical composition comprising a compound of formula (I) and pharmaceutically acceptable salts thereof for use in preventing and/or treating a disease, disorder or condition mediated by ALK5 signaling pathway in a mammal.

In a further aspect, the invention refers to a compound of formula (I) and pharmaceutically acceptable salts thereof or to a pharmaceutical composition comprising a compound of formula (I) and pharmaceutically acceptable salts thereof for use in the prevention and/or treatment of fibrosis and/or diseases, disorders, or conditions that involve fibrosis.

In a further aspect, the invention refers to a compound of formula (I) and pharmaceutically acceptable salts thereof or to a pharmaceutical composition comprising a compound of formula (I) and pharmaceutically acceptable salts thereof for use in the prevention and/or treatment idiopathic pulmonary fibrosis (IPF).

### DETAILED DESCRIPTION OF THE INVENTION

### Definitions

Unless otherwise specified, the compound of formula (I) of the present invention is intended to include also tautomer or pharmaceutically acceptable salt or solvate thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “tautomer” refers to each of two or more isomers of a compound that exist together in equilibrium and are readily interchanged by migration of an atom or group within the molecule.

The term “halogen” or “halogen atoms” or “halo” as used herein includes fluorine, chlorine, bromine, and iodine atom.

The term “(Cₓ-C_{y})alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y})alkylene” wherein x and y are integers, refers to a Cₓ-C_{y}alkyl radical having in total two unsatisfied valences, such as a divalent methylene radical.

The expressions “(Cₓ-C_{y})haloalkyl” wherein x and y are integers, refer to the above defined “Cₓ-C_{y}alkyl” groups wherein one or more hydrogen atoms are replaced by one or more halogen atoms, which can be the same or different. Examples of said “(Cₓ-C_{y})haloalkyl” groups may thus include halogenated, poly-halogenated and fully halogenated alkyl groups wherein all hydrogen atoms are replaced by halogen atoms, e.g. trifluoromethyl.

The term “(Cₓ-C_{y})cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “aryl” refers to mono cyclic carbon ring systems which have 6 ring atoms wherein the ring is aromatic. Examples of suitable aryl monocyclic ring systems include, for instance, phenyl.

The term “heteroaryl” refers to a mono- or bi-cyclic aromatic group containing one or more heteroatoms selected from S, N and O, and includes groups having two such monocyclic rings, or one such monocyclic ring and one monocyclic aryl ring, which are fused through a common bond.

The term “(Cₓ-C_{y}) heterocycloalkyl” wherein x and y are integers, refers to saturated or partially unsaturated monocyclic (Cₓ-C_{y})cycloalkyl groups in which at least one ring carbon atom is replaced by at least one heteroatom (e.g. N, S or O) or may bear an -oxo (═O) substituent group. Said heterocycloalkyl may be further optionally substituted on the available positions in the ring, namely on a carbon atom, or on an heteroatom available for substitution. Substitution on a carbon atom includes spiro disubstitution as well as substitution on two adjacent carbon atoms, in both cases thus form additional condensed 5 to 6 membered heterocyclic ring.

The term “(Cₓ-C_{y})hydroxyalkyl” wherein x and y are integers, refers to the above defined “(C₁-C₆)alkyl” groups wherein one or more hydrogen atoms are replaced by one or more hydroxy (OH) group.

Throughout the specification the use of an asterisk “*” in the definition of a structural formula, indicates the point of attachment for the radical group to the rest of the molecule.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O)—.

In general, the bracketed group is a lateral group, not included into the chain, and brackets are used, when deemed useful, to help disambiguating linear chemical formulas; e.g. the sulfonyl group —SO₂-might be also represented as —S(O)₂— to disambiguate e.g. with respect to the sulfinic group —S(O)O—.

The present invention relates to novel compounds differing from the structures disclosed in the art at least for a common new core scaffold. In fact, the invention relates to compounds that are imidazole derivatives, which are inhibitors of receptor ALK5, that have therapeutically desirable characteristics, particularly promising for some fibrosis, including idiopathic pulmonary fibrosis (IPF).

The compounds of the invention are active as inhibitors of ALK5 receptor, they are potent and show improved properties such as a good inhalatory profile, a low metabolic stability, a low systemic exposure, improved safety and tolerability.

In this respect, the state of the art does not describe or suggest imidazole derivatives of general formula (I) of the present invention having inhibitory activity on receptor ALK5 which represents a solution to the aforementioned need.

In more details, the present invention refers to a series of compounds represented by the general formula (I) as herein below described in details, which are endowed with an inhibitory activity on receptor ALK5.

Advantageously, the inhibitory action on receptor can be effective in the treatment of those diseases where these receptors play a relevant role in the pathogenesis such as fibrosis and disease, disorder and condition from fibrosis.

Differently from similar compounds of the prior art, the compounds of formula (I) of the present invention are able to act as inhibitors of ALK5 receptor, particularly appreciated by the skilled person when looking at a suitable and efficacious compounds useful for the treatment of fibrosis, in particular idiopatic pulmonary fibrosis.

As indicated in the experimental part, in particular in Table 8, the compounds of formula (I) of the present invention show a notable potency with respect to their inhibitory activity on receptor ALK5, below about 10 nM, confirming that they are able to inhibit ALK5 receptor involved in fibrosis and diseases that result from fibrosis.

Advantageously, the compounds of the present invention are endowed by a very high potency, they could be administered in human at a lower dosage respect to the compounds of the prior art, thus reducing the adverse events that typically occur administering higher dosages of drug.

In addition to being notably potent with respect to their inhibitory activity on receptor ALK5, the compounds of the present invention are also characterized by a good inhalatory profile, that permits to act effectively on the lung compartment and have, at the same time, a low metabolic stability, that allows to minimize the drawbacks associated with the systemic exposure, such as safety and tolerability issues.

Therefore, the compounds of the present invention are particularly appreciated by the skilled person when looking at a suitable and efficacious compounds useful for the treatment of fibrosis, in particular idiopatic pulmonary fibrosis, administered by the inhalation route and characterized by a good inhalatory profile, that corresponds to a good activity on the lung, a good lung retention and to a low metabolic stability, that minimizes the systemic exposure and correlated safety issues.

Thus, in one aspect the present invention relates to a compound of general formula (I) as ALK5 inhibitors

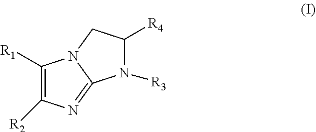

wherein
R₁ is selected from the group consisting of aryl optionally substituted by one or more groups selected from halogen atoms, NH₂, —OH, —O(C₁-C₆)alkyl, —S—(C₁-C₆)alkyl, —S(O)(C₁-C₆)alkyl, —S(O)₂—(C₁-C₆)alkyl, —S(O)₂—NH₂, —C(O)OH and —C(O)O—(C₁-C₆)alkyl; heterocycloalkyl optionally substituted by a oxo group; pyridyl substituted by NH₂; phenyl, pyridyl or thienyl fused with a structural moiety, which together with two ring members of said phenyl, pyridyl or thienyl, forms a 5-7 membered aromatic or non-aromatic ring, wherein said ring optionally contains up to three heteroatoms selected from N, O and S;
R₂ is aryl optionally substituted by one or more groups selected from halogen atoms and —(C₁-C₆)alkyl; or R₂ is heteroaryl optionally substituted by one or more groups selected from —(C₁-C₆)alkyl, —O—(C₁-C₆)alkyl, —OH and halogen atoms;
R₃ is H or is independently selected from the group consisting of —(C₁-C₆)alkyl and —C(O)—(C₁-C₆)alkyl;
R₄ is H or is selected from the group consisting of —(C₁-C₆)hydroxyalkyl, —C(O)OR₅, —C(O)O—(C₁-C₆)alkyl, —C(O)—NR₅R₆, —(C₁-C₆)alkylene-C(O)OH and —(C₁-C₆)alkylene-C(O)O—(C₁-C₆)alkyl;
R₅ is H or —(C₁-C₆)alkyl;
R₆ is —(C₁-C₆)alkyl;
and pharmaceutically acceptable salts thereof.

In a particularly preferred embodiment the present invention refers to a compound of formula (I), wherein R₁ is aryl optionally substituted by one or more groups selected from halogen atoms, NH₂, —OH, —O(C₁-C₆)alkyl, —S—(C₁-C₆)alkyl, —S(O)(C₁-C₆)alkyl, —S(O)₂—(C₁-C₆)alkyl, —S(O)₂—NH₂, —C(O)OH and —C(O)O—(C₁-C₆)alkyl;
R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H or —(C₁-C₆)alkyl;
R₄ is H;
and pharmaceutically acceptable salts thereof.

In a particularly preferred embodiment the present invention refers to a compound of formula (I), wherein R₁ is -(4-(methylthio)phenyl), -(4-(methylsulfonyl)phenyl), -(4-(methylsulfinyl)phenyl), -(3-(methylsulfinyl)phenyl), -(3-(methylsulfonyl)phenyl), -(3-(methylthio)phenyl), -3-benzensulfonamide, methyl 5-(methylthio)benzoate, methyl 2-methoxy-5-benzoate, 2-fluoro-5-aniline, 5-fluoro-2-aniline, 2-methoxy-5-benzoic acid, 2-methoxy-5-aniline, -5-(2-fluoroaniline), 2-(5-(methylthio) aniline), methyl 5-(2-(methylthio)benzoate), 5-methoxy-2-aniline, 2-(5-(2-(methylthio)benzoic acid), 5-(2-(methylthio) aniline) and 4-phenol.

According to a preferred embodiment, the invention refers to at least one of the compounds of Formula (I) listed in the Table 1 below and pharmaceutically acceptable salts thereof.

TABLE 1
List of preferred compounds of Formula (I)

| Example | | |
|---|---|---|
| No. | Structure | Chemical Name |
| 23 | 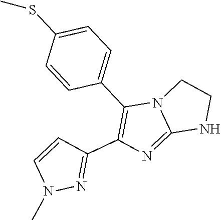 [SMILES: CSc1ccc(-c2c(-c3ccn(C)n3)nc3n2CCN3)cc1] | 6-(1-Methyl-1H-pyrazol-3-yl)- 5-(4-(methylthio)phenyl)-2,3- dihydro-1H-imidazo[1,2- a]imidazole |
| 32 | 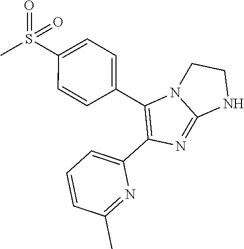 [SMILES: Cc1cccc(-c2nc3n(c2-c2ccc(S(C)(=O)=O)cc2)CCN3)n1] | 6-(6-methylpyridin-2-yl)-5-(4- (methylsulfonyl)phenyl)-2,3- dihydro-1H-imidazo[1,2- a]imidazole |
| 34 | 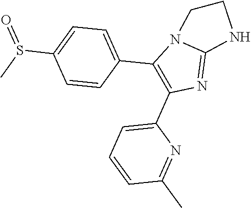 [SMILES: Cc1cccc(-c2nc3n(c2-c2ccc(S(C)=O)cc2)CCN3)n1] | 6-(6-Methylpyridin-2-yl)-5-(4- (methylsulfinyl)phenyl)-2,3- dihydro-1H-imidazo[1,2- a]imidazole |
| 36 | 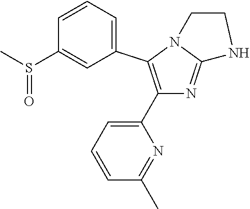 [SMILES: Cc1cccc(-c2nc3n(c2-c2cccc(S(C)=O)c2)CCN3)n1] | 6-(6-Methylpyridin-2-yl)-5-(3- (methylsulfinyl)phenyl)-2,3- dihydro-1H-imidazo[1,2- a]imidazole |
| 37 | 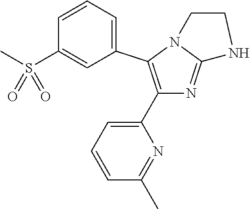 [SMILES: Cc1cccc(-c2nc3n(c2-c2cccc(S(C)(=O)=O)c2)CCN3)n1] | 1-6-(6-Methylpyridin-2-yl)-5- (3-(methylsulfonyl)phenyl)-2,3- dihydro-1H-imidazo[1,2- a]imidazole |
| 38 | 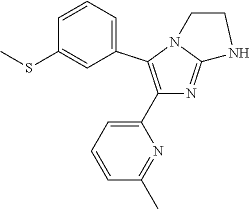 [SMILES: CSc1cccc(-c2c(-c3cccc(C)n3)nc3n2CCN3)c1] | 6-(6-Methylpyridin-2-yl)-5-(3- (methylthio)phenyl)-2,3- dihydro-1H-imidazo[1,2- a]imidazole |
| 39 | 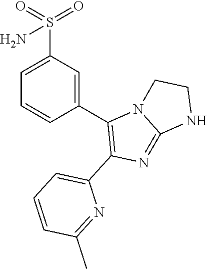 [SMILES: Cc1cccc(-c2nc3n(c2-c2cccc(S(N)(=O)=O)c2)CCN3)n1] | 3-(6-(6-Methylpyridin-2-yl)- 2,3-dihydro-1H-imidazo[1,2- a]imidazol-5- yl)benzenesulfonamide |
| 40 | 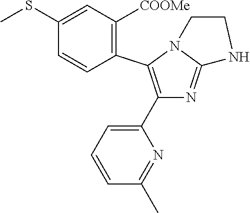 [SMILES: COC(=O)c1cc(SC)ccc1-c1c(-c2cccc(C)n2)nc2n1CCN2] | Methyl 2-(6-(6-methylpyridin- 2-yl)-2,3-dihydro-1H- imidazo[1,2-a]imidazol-5-yl)-5- (methylthio)benzoate |
| 41 | 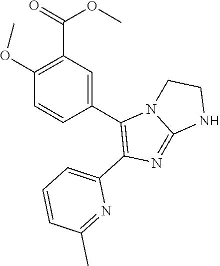 [SMILES: COC(=O)c1cc(-c2c(-c3cccc(C)n3)nc3n2CCN3)ccc1OC] | Methyl 2-methoxy-5-(6-(6- methylpyridin-2-yl)-2,3- dihydro-1H-imidazo[1,2- a]imidazol-5-yl)benzoate |
| 43 | 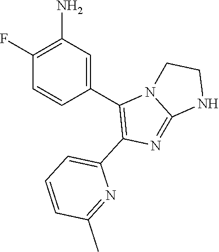 [SMILES: Cc1cccc(-c2nc3n(c2-c2ccc(F)c(N)c2)CCN3)n1] | 2-Fluoro-5-(6-(6-methylpyridin- 2-yl)-2,3-dihydro-1H- imidazo[1,2-a]imidazol-5- yl)aniline |
| 44 | 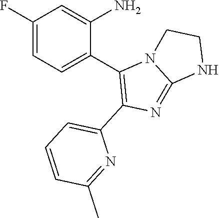 [SMILES: Cc1cccc(-c2nc3n(c2-c2ccc(F)cc2N)CCN3)n1] | 5-Fluoro-2-(6-(6-methylpyridin- 2-yl)-2,3-dihydro-1H- imidazo[1,2-a]imidazol-5- yl)aniline |
| 45 | 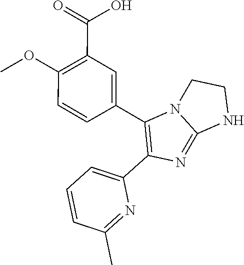 [SMILES: COc1ccc(-c2c(-c3cccc(C)n3)nc3n2CCN3)cc1C(=O)O] | 2-Methoxy-5-(6-(6- methylpyridin-2-yl)-2,3- dihydro-1H-imidazo[1,2- a]imidazol-5-yl)benzoic acid |
| 46 | 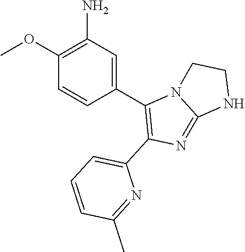 [SMILES: COc1ccc(-c2c(-c3cccc(C)n3)nc3n2CCN3)cc1N] | 2-Methoxy-5-(6-(6- methylpyridin-2-yl)-2,3- dihydro-1H-imidazo[1,2- a]imidazol-5-yl)aniline |
| 47 | 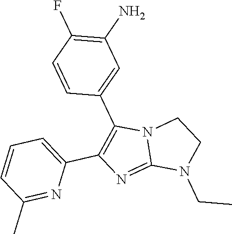 [SMILES: CCN1CCn2c1nc(-c1cccc(C)n1)c2-c1ccc(F)c(N)c1] | 5-(1-Ethyl-6-(6-methylpyridin- 2-yl)-2,3-dihydro-1H- imidazo[1,2-a]imidazol-5-yl)-2- fluoroaniline |
| 49 | 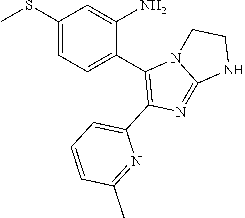 [SMILES: CSc1ccc(-c2c(-c3cccc(C)n3)nc3n2CCN3)c(N)c1] | 2-(6-(6-Methylpyridin-2-yl)- 2,3-dihydro-1H-imidazo[1,2- alimidazol-5-yl)-5- (methylthio)aniline |
| 50 | 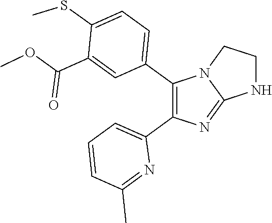 [SMILES: COC(=O)c1cc(-c2c(-c3cccc(C)n3)nc3n2CCN3)ccc1SC] | Methyl 5-(6-(6-methylpyridin- 2-yl)-2,3-dihydro-1H- imidazo[1,2-a]imidazol-5-yl)-2- (methylthio)benzoate |
| 51 | 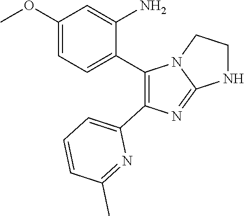 [SMILES: COc1ccc(-c2c(-c3cccc(C)n3)nc3n2CCN3)c(N)c1] | 5-Methoxy-2-(6-(6- methylpyridin-2-yl)-2,3- dihydro-1H-imidazo[1,2- a]imidazol-5-yl)aniline |
| 52 | 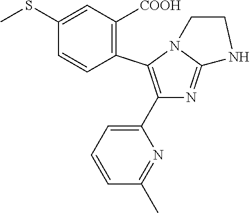 [SMILES: CSc1ccc(-c2c(-c3cccc(C)n3)nc3n2CCN3)c(C(=O)O)c1] | 2-(6-(6-methylpyridin-2-yl)- 2,3-dihydro-1H-imidazo[1,2- a]imidazol-5-yl)-5- (methylthio)benzoic acid |
| 54 | 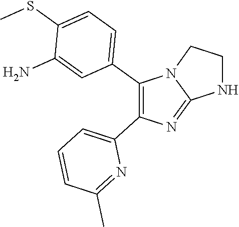 [SMILES: CSc1ccc(-c2c(-c3cccc(C)n3)nc3n2CCN3)cc1N] | 5-(6-(6-Methylpyridin-2-yl)- 2,3-dihydro-1H-imidazo[1,2- a]imidazol-5-yl)-2- (methylthio)aniline |
| 55 | 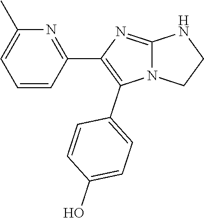 [SMILES: Cc1cccc(-c2nc3n(c2-c2ccc(O)cc2)CCN3)n1] | 4-[6-(6-Methylpyridin-2-yl)- 1H,2H,3H-imidazo[1,2- a][1,3]diazol-5-yl]phenol |

In another preferred embodiment the present invention refers to a compound of formula (I), wherein R1 is phenyl fused with a structural moiety, which together with two ring members of said phenyl forms a 5-7 membered aromatic or non-aromatic ring, wherein said ring optionally contains up to three heteroatoms selected from N, O and S;

R2 is aryl optionally substituted by one or more groups selected from halogen atoms and —(C1-C6)alkyl; or R2 is heteroaryl optionally substituted by one or more groups selected from —(C1-C6)alkyl, —O(C1-C6)alkyl, —OH and halogen atoms;

and pharmaceutically acceptable salts thereof.

In a particularly preferred embodiment the present invention refers to a compound of formula (I), wherein R1 is 4-(benzo[c][1,2,5] thiadiazole), 4-benzo[c][1,2,5] oxadiazole, 1-benzo[d] thiazole, 7-benzo[d][1,2,3] thiadiazole, 5-benzo[c][1,2,5] thiadiazole, 5-benzo[d][1,2,3] oxadiazole, -(benzo[d][1,3]dioxol-5-yl), 4-benzo[d][1,2,3] thiadiazole, 5-benzo[d][1,2,3] thiadiazole, 6-benzo[d][1,2,3] thiadiazole and 6-(1,3-benzothiazole).

According to a preferred embodiment, the invention refers to at least one of the compounds of Formula (I) listed in the Table 2 below and pharmaceutically acceptable salts thereof.

TABLE 2
List of preferred compounds of Formula (I)

| Example | | |
|---|---|---|
| No. | Structure | Chemical Name |
| 10 | 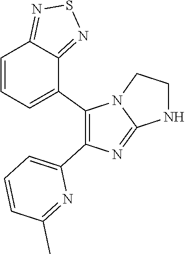 [SMILES: Cc1cccc(-c2nc3n(c2-c2cccc4nsnc24)CCN3)n1] | 4-(6-(6-Methylpyridin-2-yl)-2,3- dihydro-1H-imidazo[1,2-a]imidazol- 5-yl)benzo[c][1,2,5]thiadiazole |
| 11 | 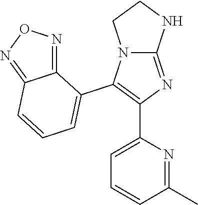 [SMILES: Cc1cccc(-c2nc3n(c2-c2cccc4nonc24)CCN3)n1] | 4-(6-(6-methylpyridin-2-yl)-2,3- dihydro-1H-imidazo[1,2-a]imidazol- 5-yl)benzo[c][1,2,5]oxadiazole |
| 15 | 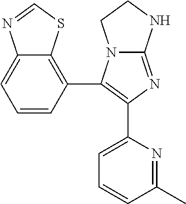 [SMILES: Cc1cccc(-c2nc3n(c2-c2cccc4ncsc24)CCN3)n1] | 1-(6-(6-Methylpyridin-2-yl)-2,3- dihydro-1H-imidazo[1,2-a]imidazol- 5-yl)benzo[d]thiazole |
| 16 | 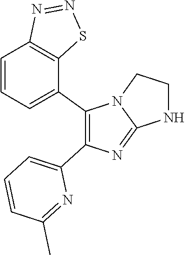 [SMILES: Cc1cccc(-c2nc3n(c2-c2cccc4nnsc24)CCN3)n1] | 7-(6-(6-Methylpyridin-2-yl)-2,3- dihydro-1H-imidazo[1,2-a]imidazol- 5-yl)benzo[d][1,2,3 ]thiadiazole |
| 17 | 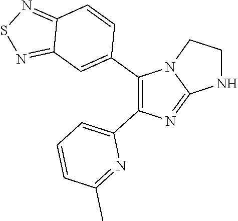 [SMILES: Cc1cccc(-c2nc3n(c2-c2ccc4nsnc4c2)CCN3)n1] | 5-(6-(6-Methylpyridin-2-yl)-2,3- dihydro-1H-imidazo[1,2-a]imidazol- 5-yl)benzo[c][1,2,5]thiadiazole |
| 18 | 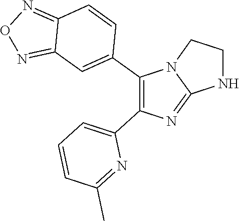 [SMILES: Cc1cccc(-c2nc3n(c2-c2ccc4nonc4c2)CCN3)n1] | 5-(6-(6-methylpyridin-2-yl)-2,3- dihydro-1H-imidazo[1,2-a]imidazol- 5-yl)benzo[c][1,2,5]oxadiazole |
| 24 | 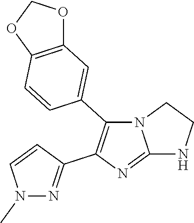 [SMILES: Cn1ccc(-c2nc3n(c2-c2ccc4c(c2)OCO4)CCN3)n1] | 5-(Benzo[d][1,3]dioxol-5-yl)-6-(1- methyl-1H-pyrazol-3-yl)-2,3- dihydro-1H-imidazo[1,2- a]imidazole |
| 28 | 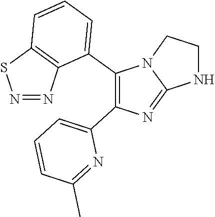 [SMILES: Cc1cccc(-c2nc3n(c2-c2cccc4snnc24)CCN3)n1] | 4-(6-(6-Methylpyridin-2-yl)-2,3- dihydro-1H-imidazo[1,2-a]imidazol- 5-yl)benzo[d][1,2,3]thiadiazole |
| 30 | 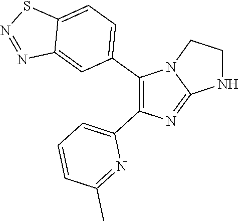 [SMILES: Cc1cccc(-c2nc3n(c2-c2ccc4snnc4c2)CCN3)n1] | 5-(6-(6-Methylpyridin-2-yl)-2,3- dihydro-1H-imidazo[1,2-a]imidazol- 5-yl)benzo[d][1,2,3]thiadiazole |
| 31 | 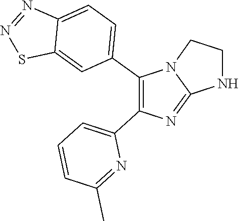 [SMILES: Cc1cccc(-c2nc3n(c2-c2ccc4nnsc4c2)CCN3)n1] | 6-(6-(6-Methylpyridin-2-yl)-2,3- dihydro-1H-imidazo[1,2-a]imidazol- 5-yl)benzo[d][1,2,3 ]thiadiazole |
| 33 | 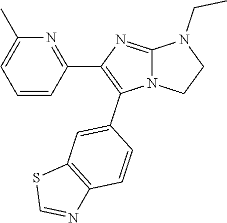 [SMILES: CCN1CCn2c1nc(-c1cccc(C)n1)c2-c1ccc2ncsc2c1] | 6-[1-Ethyl-6-(6-methylpyridin-2-yl)- 1H,2H,3H-imidazo[1,2- a][1,3]diazol-5-yl]-1,3- benzothiazole |
| 57 | 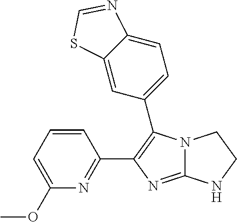 [SMILES: COc1cccc(-c2nc3n(c2-c2ccc4ncsc4c2)CCN3)n1] | 6-[6-(6-Methoxypyridin-2-yl)- 1H,2H,3H-imidazo[1,2- a][1,3]diazol-5-yl]-1,3- benzothiazole |
| 58 | 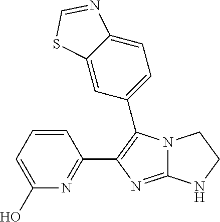 [SMILES: Oc1cccc(-c2nc3n(c2-c2ccc4ncsc4c2)CCN3)n1] | 6-[5-(1,3-Benzothiazol-6-yl)- 1H,2H,3H-imidazo[1,2- a][1,3]diazol-6-yl]pyridin-2-ol |

In another preferred embodiment, the present invention refers to a compound of formula (I), wherein R₁ is pyridyl substituted by NH₂; R₂ is heteroaryl optionally substituted by —(C₁-C₆)alkyl; and pharmaceutically acceptable salts thereof.

In an even further preferred embodiment, the present invention refers to a compound of formula (I), wherein R₁ is 4-pyridin-2-amine.

According to a preferred embodiment, the invention refers to a compound of Formula (I) listed in the Table 9 below and pharmaceutically acceptable salts thereof.

TABLE 9
List of preferred compounds of Formula (I)

| Example | | |
|---|---|---|
| No. | Structure | Chemical Name |
| 56 | 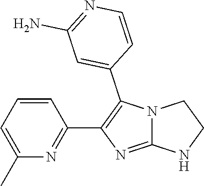 [SMILES: Cc1cccc(-c2nc3n(c2-c2ccnc(N)c2)CCN3)n1] | 4-[6-(6-Methylpyridin-2-yl)- 1H,2H,3H-imidazo[1,2- a][1,3]diazol-5-yl]pyridin-2-amine |

In a further preferred embodiment, the present invention refers to a compound of formula (I), wherein R₁ is pyridyl fused with a structural moiety, which together with two ring members of said pyridyl forms a 5-7 membered aromatic or non-aromatic ring, wherein said ring optionally contains up to three heteroatoms selected from N, O and S;
R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H or is independently selected from the group consisting of —(C₁-C₆)alkyl and —C(O)—(C₁-C₆)alkyl;
R₄ is H or is selected from the group consisting of —(C₁-C₆)hydroxyalkyl and —C(O)OR₅; and pharmaceutically acceptable salts thereof.

In a particularly preferred embodiment the present invention refers to a compound of formula (I), wherein R₁ is -([1,2,4]triazolo[1,5-a]pyridin-6-yl) and 4 (-1H-pyrrolo[2,3-b]pyridine).

According to a preferred embodiment, the invention refers to at least one of the compounds of Formula (I) listed in the Table 3 below and pharmaceutically acceptable salts thereof.

TABLE 3
List of preferred compounds of Formula (I)

| Example | | |
|---|---|---|
| No. | Structure | Chemical Name |
| 2 | 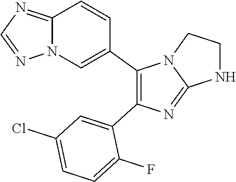 [SMILES: Fc1ccc(Cl)cc1-c1nc2n(c1-c1ccc3ncnn3c1)CCN2] | 6-(6-(5-Chloro-2-fluorophenyl)-2,3- dihydro-1H-imidazo[1,2-a]imidazol- 5-yl)-[1,2,4]triazolo[1,5-a]pyridine |
| 3 | 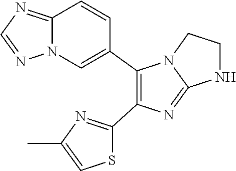 [SMILES: Cc1csc(-c2nc3n(c2-c2ccc4ncnn4c2)CCN3)n1] | 2-(5-([1,2,4]Triazolo[1,5-a]pyridin- 6-yl)-2,3-dihydro-1H-imidazo[1,2- a]imidazol-6-yl)-4-methylthiazole |
| 8 | 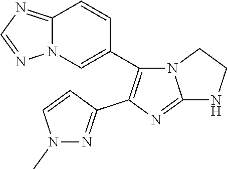 [SMILES: Cn1ccc(-c2nc3n(c2-c2ccc4ncnn4c2)CCN3)n1] | 6-(6-(1-Methyl-1H-pyrazol-3-yl)- 2,3-dihydro-1H-imidazo[1,2- a]imidazol-5-yl)-[1,2,4]triazolo[1,5- a]pyridine |
| 12 | 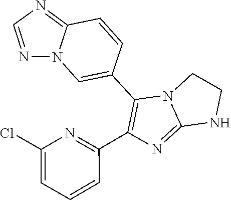 [SMILES: Clc1cccc(-c2nc3n(c2-c2ccc4ncnn4c2)CCN3)n1] | 6-(6-(6-Chloropyridin-2-yl)-2,3- dihydro-1H-imidazo[1,2-a]imidazol- 5-yl)-[1,2,4]triazolo[1,5-a]pyridine |
| 22 | 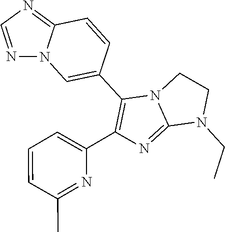 [SMILES: CCN1CCn2c1nc(-c1cccc(C)n1)c2-c1ccc2ncnn2c1] | 6-(1-Ethyl-6-(6-methylpyridin-2-yl)- 2,3-dihydro-1H-imidazo[1,2- a]imidazol-5-yl)-[1,2,4]triazolo[1,5- a]pyridine |
| 25 | 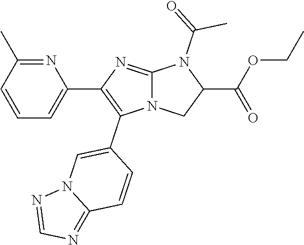 [SMILES: CCOC(=O)C1Cn2c(nc(-c3cccc(C)n3)c2-c2ccc3ncnn3c2)N1C(C)=O] | Ethyl 1-acetyl-6-(6-methylpyridin- 2-yl)-5-{[1,2,4]triazolo[1,5- alpyridin-6-yl}-1H,2H,3H- imidazo[1,2-a][1,3]diazole-2- carboxylate |
| 26 | 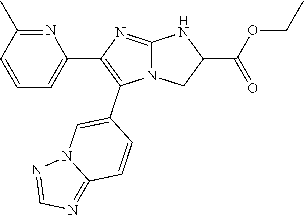 [SMILES: CCOC(=O)C1Cn2c(nc(-c3cccc(C)n3)c2-c2ccc3ncnn3c2)N1] | Ethyl 6-(6-methylpyridin-2-yl)-5- {[1,2,4]triazolo[1,5-a]pyridin-6-yl}- 1H,2H,3H-imidazo[1,2- a][1,3]diazole-2-carboxylate |
| 27 | 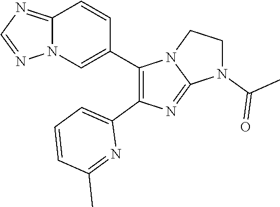 [SMILES: CC(=O)N1CCn2c1nc(-c1cccc(C)n1)c2-c1ccc2ncnn2c1] | 1-(5-([1,2,4]Triazolo[1,5-a]pyridin- 6-yl)-6-(6-methylpyridin-2-yl)-2,3- dihydro-1H-imidazo[1,2-a]imidazol- 1-yl)ethan-1-one |
| 35 | 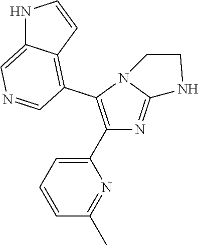 [SMILES: Cc1cccc(-c2nc3n(c2-c2cncc4[nH]ccc24)CCN3)n1] | 4-(6-(6-Methylpyridin-2-yl)-2,3- dihydro-1H-imidazo[1,2-a]imidazol- 5-yl)-1H-pyrrolo[2,3-b ]pyridine |

In another prefererd embodiment, the present invention relates to a compound of general formula (I), wherein R₁ is group R₁ₓ

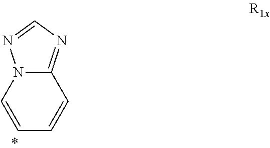

represented by the formula (Ix)

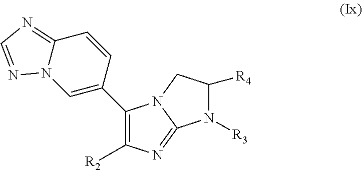

R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H or ethyl;
R₄ is H;
and pharmaceutically acceptable salts thereof.

According to a preferred embodiment, the invention refers to at least one of the compounds of Formula (Ix) listed in the Table 4 below and pharmaceutical acceptable salts thereof. These compounds are particularly active on receptor ALK5, as shown in Table 8.

TABLE 4
List of preferred compounds of Formula (Ix)

| Example | | |
|---|---|---|
| No. | Structure | Chemical Name |
| 2 | 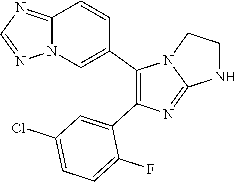 [SMILES: Fc1ccc(Cl)cc1-c1nc2n(c1-c1ccc3ncnn3c1)CCN2] | 6-(6-(5-Chloro-2-fluorophenyl)-2,3- dihydro-1H-imidazo[1,2-a]imidazol- 5-yl)-[1,2,4]triazolo[1,5-a]pyridine |
| 3 | 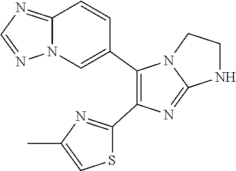 [SMILES: Cc1csc(-c2nc3n(c2-c2ccc4ncnn4c2)CCN3)n1] | 2-(5-([1,2,4]Triazolo[1,5-a]pyridin- 6-yl)-2,3-dihydro-1H-imidazo[1,2- a]imidazol-6-yl)-4-methylthiazole |
| 12 | 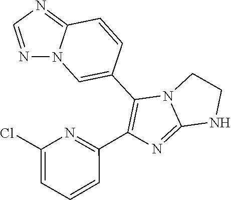 [SMILES: Clc1cccc(-c2nc3n(c2-c2ccc4ncnn4c2)CCN3)n1] | 6-(6-(6-Chloropyridin-2-yl)-2,3- dihydro-1H-imidazo[1,2-a]imidazol- 5-yl)-[1,2,4]triazolo[1,5-a]pyridine |
| 22 | 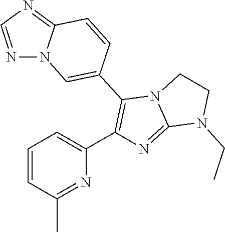 [SMILES: CCN1CCn2c1nc(-c1cccc(C)n1)c2-c1ccc2ncnn2c1] | 6-(1-Ethyl-6-(6-methylpyridin-2-yl)- 2,3-dihydro-1H-imidazo[1,2- a]imidazol-5-yl)-[1,2,4]triazolo[1,5- alpyridine |

In an even further prefererd embodiment, the present invention relates to a compound of general formula (I), wherein R₁ is thienyl fused with a structural moiety, which together with two ring members of said thienyl forms a 5-7 membered aromatic or non-aromatic ring, wherein said ring optionally contains up to three heteroatoms selected from N, O and S;
R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H or —(C₁-C₆)alkyl;
R₄ is H or is selected from the group consisting of —(C₁-C₆)hydroxyalkyl, —C(O)OR₅, —C(O)—NR₅R₆, —(C₁-C₆)alkylene-C(O)OH and —(C₁-C₆)alkylene-C(O)O—(C₁-C₆)alkyl;
R₅ is H or —(C₁-C₆)alkyl;
R₆ is —(C₁-C₆)alkyl;
   and pharmaceutically acceptable salts thereof.

In a particularly preferred embodiment the present invention refers to a compound of formula (I), wherein R₁ is -5-{thieno[3,2-c]pyridin-2-yl}.

According to a preferred embodiment, the invention refers to at least one of the compounds of Formula (I) listed in the Table 5 below and pharmaceutically acceptable salts thereof.

TABLE 5
List of preferred compounds of Formula (I)

| Example | | |
|---|---|---|
| No. | Structure | Chemical Name |
| 1 | 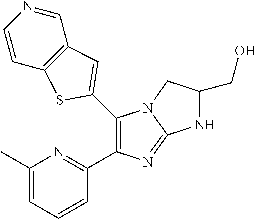 [SMILES: Cc1cccc(-c2nc3n(c2-c2cc4cnccc4s2)CC(CO)N3)n1] | [6-(6-Methylpyridin-2-yl)-5- {thieno[3,2-c]pyridin-2-yl}- 1H,2H,3H-imidazo[1,2- a][1,3]diazol-2-yl]methanol, single enantiomer |
| 4 | 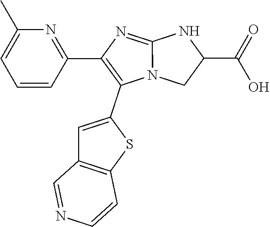 [SMILES: Cc1cccc(-c2nc3n(c2-c2cc4cnccc4s2)CC(C(=O)O)N3)n1] | 6-(6-Methylpyridin-2-yl)-5- {thieno[3,2-c]pyridin-2-yl}- 1H,2H,3H-imidazo[1,2- a][1,3]diazole-2-carboxylic acid |
| 7 | 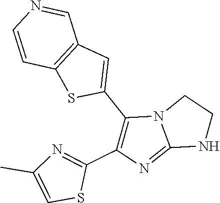 [SMILES: Cc1csc(-c2nc3n(c2-c2cc4cnccc4s2)CCN3)n1] | 2-(6-(4-Methylthiazol-2-yl)-2,3- dihydro-1H-imidazo[1,2-a]imidazol- 5-yl)thieno[3,2-c]pyridine |
| 9 | 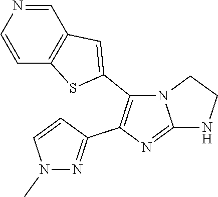 [SMILES: Cn1ccc(-c2nc3n(c2-c2cc4cnccc4s2)CCN3)n1] | 2-(6-(1-Methyl-1H-pyrazol-3-yl)- 2,3-dihydro-1H-imidazo[1,2- a]imidazol-5-yl)thieno[3,2- c]pyridine |
| 13 | 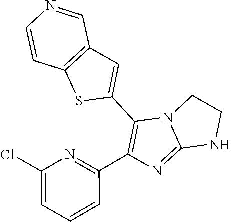 [SMILES: Clc1cccc(-c2nc3n(c2-c2cc4cnccc4s2)CCN3)n1] | 2-(6-(6-Chloropyridin-2-yl)-2,3- dihydro-1H-imidazo[1,2-a]imidazol- 5-yl)thieno[3,2-c]pyridine |
| 14 | 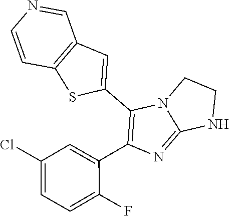 [SMILES: Fc1ccc(Cl)cc1-c1nc2n(c1-c1cc3cnccc3s1)CCN2] | 2-(6-(5-Chloro-2-fluorophenyl)-2,3- dihydro-1H-imidazo[1,2-a]imidazol- 5-yl)thieno[3,2-c]pyridine |
| 19 | 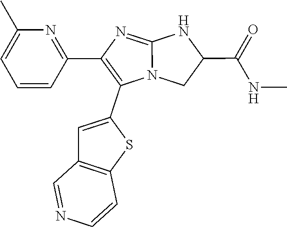 | N-methyl-6-(6-methylpyridin-2-yl)- 5-{thieno[3,2-b]pyridin-2-yl}- 1H,2H,3H-imidazo[1,2- a][1,3]diazole-2-carboxamide |
| 20 | 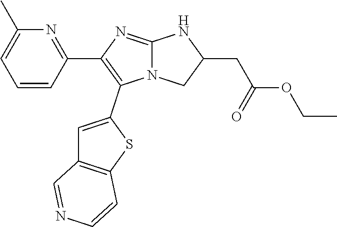 [SMILES: CCOC(=O)CC1Cn2c(nc(-c3cccc(C)n3)c2-c2cc3cnccc3s2)N1] | Ethyl 2-[6-(6-methylpyridin-2-yl)-5- {thieno[3,2-c]pyridin-2-yl}- 1H,2H,3H-imidazo[1,2- a][1,3]diazol-2-yl]acetate |
| 21 | 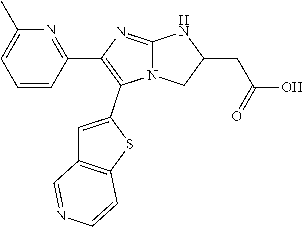 [SMILES: Cc1cccc(-c2nc3n(c2-c2cc4cnccc4s2)CC(CC(=O)O)N3)n1] | 2-[6-(6-Methylpyridin-2-yl)-5- {thieno[3,2-c]pyridin-2-yl}- 1H,2H,3H-imidazo[1,2- a][1,3]diazol-2-yl]acetic acid |
| 29 | 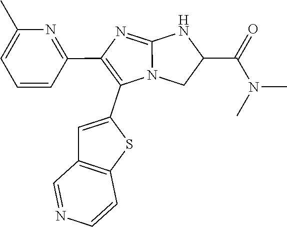 | N,N-Dimethyl-6-(6-methylpyridin- 2-yl)-5-{thieno[3,2-c]pyridin-2-yl}- 1H,2H,3H-imidazo[1,2- a][1,3]diazole-2-carboxamide |
| 42 | 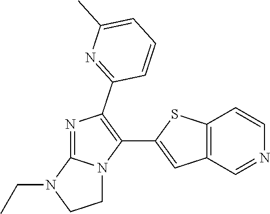 [SMILES: CCN1CCn2c1nc(-c1cccc(C)n1)c2-c1cc2cnccc2s1] | 2-(1-Ethyl-5-{thieno[3,2-c]pyridin- 2-yl}-1H,2H,3H-imidazo[1,2- a][1,3]diazol-6-yl)-6-methylpyridine |

In a even further prefererd embodiment, the present invention relates to a compound of general formula (I), wherein R₁ is group R_{1y}

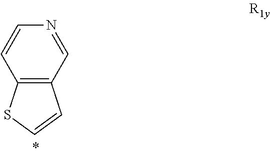

represented by the formula (Iy)

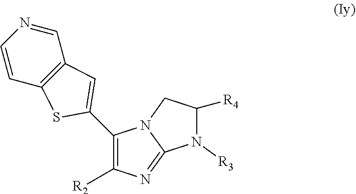

R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H;
R₄ is H or is selected from the group consisting of hydroxymethyl, —C(O)OH, N-methylacetamide;
and pharmaceutically acceptable salts thereof.

According to a preferred embodiment, the invention refers to at least one of the compounds of Formula (Iy) listed in the Table 6 below and pharmaceutical acceptable salts thereof. These compounds are particularly active on receptor ALK5, as shown in Table 8.

TABLE 6
List of preferred compounds of Formula (ly)

| Example | | |
|---|---|---|
| No. | Structure | Chemical Name |
| 1 | 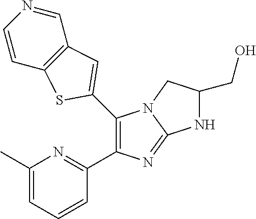 [SMILES: Cc1cccc(-c2nc3n(c2-c2cc4cnccc4s2)CC(CO)N3)n1] | [6-(6-Methylpyridin-2-yl)-5- {thieno[3,2-c]pyridin-2-yl}- 1H,2H,3H-imidazo[1,2- a][1,3]diazol-2-yl]methanol, single enantiomer |
| 4 | 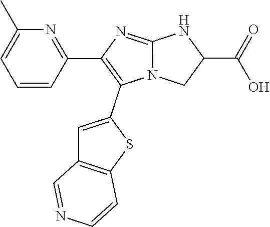 [SMILES: Cc1cccc(-c2nc3n(c2-c2cc4cnccc4s2)CC(C(=O)O)N3)n1] | 6-(6-Methylpyridin-2-yl)-5- {thieno[3,2-c]pyridin-2-yl}- 1H,2H,3H-imidazo[1,2- a][1,3]diazole-2-carboxylic acid |
| 7 | 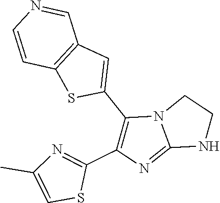 [SMILES: Cc1csc(-c2nc3n(c2-c2cc4cnccc4s2)CCN3)n1] | 2-(6-(4-Methylthiazol-2-yl)-2,3- dihydro-1H-imidazo[1,2-a]imidazol- 5-yl)thieno[3,2-c]pyridine |
| 13 | 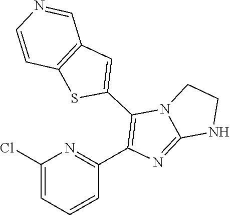 [SMILES: Clc1cccc(-c2nc3n(c2-c2cc4cnccc4s2)CCN3)n1] | 2-(6-(6-Chloropyridin-2-yl)-2,3- dihydro-1H-imidazo[1,2-a]imidazol- 5-yl)thieno[3,2-c]pyridine |
| 14 | 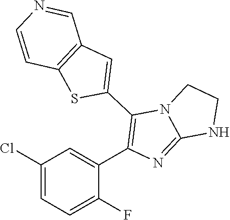 [SMILES: Fc1ccc(Cl)cc1-c1nc2n(c1-c1cc3cnccc3s1)CCN2] | 2-(6-(5-Chloro-2-fluorophenyl)-2,3- dihydro-1H-imidazo[1,2-a]imidazol- 5-yl)thieno[3,2-c]pyridine |
| 19 | 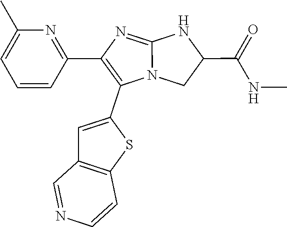 | N-methyl-6-(6-methylpyridin-2-yl)- 5-{thieno[3,2-b]pyridin-2-yl}- 1H,2H,3H-imidazo[1,2- a][1,3]diazole-2-carboxamide |

In another embodiment, the present invention relates to a compound of formula (I), wherein R₁ is heterocycloalkyl optionally substituted by an oxo group;
R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₅ and R₄ are H;
and pharmaceutically acceptable salts thereof.

In a particularly preferred embodiment the present invention refers to a compound of formula (I), wherein R₁ is 4-2H-pyran-2-one and 5-2H-pyran-2one.

According to a preferred embodiment, the invention refers to at least one of the compounds of Formula (I) listed in the Table 7 below and pharmaceutically acceptable salts thereof.

TABLE 7
List of preferred compounds of Formula (I)

| Example | | |
|---|---|---|
| No. | Structure | Chemical Name |
| 5 | 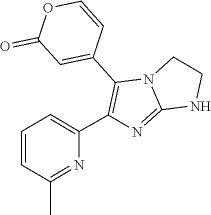 [SMILES: Cc1cccc(-c2nc3n(c2-c2ccoc(=O)c2)CCN3)n1] | 4-(6-(6-Methylpyridin-2-yl)-2,3- dihydro-1H-imidazo[1,2-a]imidazol- 5-yl)-2H-pyran-2-one |
| 6 | 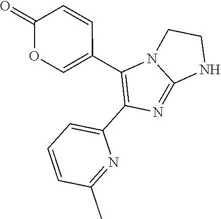 [SMILES: Cc1cccc(-c2nc3n(c2-c2ccc(=O)oc2)CCN3)n1] | 5-(6-(6-Methylpyridin-2-yl)-2,3- dihydro-1H-imidazo[1,2-a]imidazol- 5-yl)-2H-pyran-2-one |

In an even further preferred embodiment, the present invention relates to a compound of formula (I), wherein R₂ is phenyl optionally substituted by one or more groups selected from fluorine and chlorine; pyridinyl optionally substituted by one or more groups selected from methyl and chlorine; thiazolyl optionally substituted by one or more methyl; and pyrazolyl optionally substituted by one or more methyl.

The compounds of formula (I) of the present invention have surprisingly been found to effectively inhibit the receptor ALK5. Advantageously, the inhibition of ALK5 may result in efficacious treatment of the diseases or condition wherein the ALK5 signaling is involved.

In this respect, it has now been found that the compounds of formula (I) of the present invention have an inhibitory drug potency expressed as half maximal inhibitory concentration (IC50) on ALK5 lower or equal than 10 nM as shown in the present experimental part. Preferably, the compounds of the present invention have an IC50 on ALK5 between 5 and 10 nM. Even more preferably, the compounds of the present invention have an IC50 on ALK5 lower than 1 nM.

In one aspect, the present invention refers to a compound of formula (I) or a pharmaceutically acceptable salt thereof, for use as a medicament.

In a further aspect, the invention refers to the use of a compound of formula (I) of the invention, or a pharmaceutically acceptable salt thereof, in the manufacture of a medicament for the prevention and/or treatment of a disease, disorder or condition associated with dysregulated ALK5 signaling pathway.

In a preferred embodiment, the invention refers to a compound of formula (I) or a pharmaceutically acceptable salt thereof, for use in the prevention and/or treatment of a disease, disorder or condition associated with dysregulated ALK5 signaling pathway. In one embodiment, the present invention refers to a compound of formula (I) useful for the prevention and/or treatment of fibrosis and/or diseases, disorders, or conditions that involve fibrosis.

The invention also provides a method for the prevention and/or treatment of a disease, disorder or condition associated with dysregulated ALK5 signaling pathway, said method comprising administering to a patient in need of such treatment a therapeutically effective amount of a compound of the invention.

In particular the invention refers to a method for the prevention and/or treatment of fibrosis and/or diseases, disorders, or conditions that involve fibrosis, wherein said method comprises the administration of a proper amount of a compound of formula (I) to a patient in the need thereof.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract. Preferably, the compounds of formula (I) of the present invention, or a pharmaceutical composition comprising a compound of formula (I), are useful for the treatment and/or prevention of fibrosis such as pulmonary fibrosis, idiopathic pulmonary fibrosis (IPF), hepatic fibrosis, renal fibrosis, ocular fibrosis, cardiac fibrosis, arterial fibrosis and systemic sclerosis.

More preferably, the compounds of formula (I) of the present invention, or a pharmaceutical composition comprising a compound of formula (I), are useful for the treatment of idiopathic pulmonary fibrosis (IPF).

The methods of treatment of the invention comprise administering a safe and effective amount of a compound of formula (I) or a pharmaceutically acceptable salt thereof to a patient in need thereof. As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan. The compounds of formula (I) or pharmaceutically acceptable salts thereof may be administered once or according to a dosing regimen wherein a number of doses are administered at varying intervals of time for a given period of time. Typical daily dosages may vary depending upon the particular route of administration chosen.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation. More preferably, the compounds of the present invention are administered by inhalation.

In one preferred embodiment, the pharmaceutical composition comprising the compound of formula (I) is a solid oral dosage form such as tablets, gelcaps, capsules, caplets, granules, lozenges and bulk powders. The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In one embodiment, the pharmaceutical composition comprising the compound of formula (I) is a tablet.

In a further embodiment, the pharmaceutical composition comprising a compound of formula (I) is a liquid oral dosage forms such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention. The compounds of the invention may be injected, for example, intravenously, in the form of an isotonic sterile solution.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention are administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of formula (I) according to the invention, in form of a single- or multi-dose dry powder inhaler or a metered dose inhaler.

All preferred groups or embodiments described above for compounds of formula (I) may be combined among each other and apply as well mutatis mutandis.

The various aspects of the invention described in this application are illustrated by the following examples which are not meant to limit the invention in any way.

The compounds of the invention, including all the compounds here above listed, can be prepared from readily available starting materials using the following general methods and procedures or by using slightly modified processes readily available to those of ordinary skill in the art. Although a particular embodiment of the present invention may be shown or described herein, those skilled in the art will recognize that all embodiments or aspects of the present invention can be obtained using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures.

Thus, processes described below and reported in the following schemes should not be viewed as limiting the scope of the synthetic methods available for the preparation of the compounds of the invention.

The compounds of formula (I) including all the compounds or at least one of the here above listed can be generally prepared according to the procedure outlined in detail in the Schemes shown below, using generally known methods.

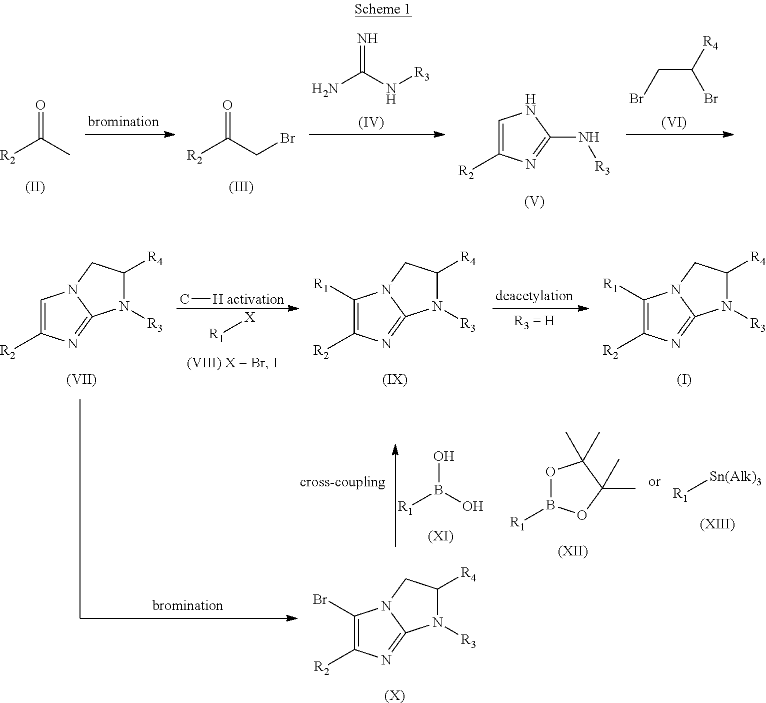

In one embodiment of the present invention, wherein R₁ and R₂ are defined as above, R₃ is H, and R₄ is H or selected from the groups —C(O)OR₅ or —(C₁-C₆)alkylene-C(O)OR₅, compounds of formula (I) may be prepared as described in Scheme 1, starting from commercially available compound (II), wherein R₂ is defined as above. Compound (III) may be prepared by bromination of compound (II). The reagents suitable for this reaction are, but not limited to, bromine and hydrogen bromide in acetic acid. Compounds of formula (V) may be obtained by cyclization reaction between compound (III) and commercially available compound (IV), wherein R₃ is —C(O)—(C₁-C₆)alkyl, typically acetyl group. Cyclization conditions include a suitable base, such as K₂CO₃, in a polar aprotic solvent, as for example MeCN and the like, and at an appropriate temperature, for example, 50° C. Compounds (VI), wherein R₄ is H or selected from the groups —C(O)OR₅ and —(C₁-C₆)alkylene-C(O)OR₅, can be commercially available and employed in a cyclization reaction with compounds (V) to yield compounds of formula (VII). Cyclization conditions comprise the use of a proper base, such as for example K₂CO₃, a suitable solvent as MeCN and an appropriate temperature as, for instance, 80° C.

A compound of formula (VII) can undergo a metal-catalyzed C—H activation coupling with suitable halides of formula (VIII) wherein R₁ is defined as above, to give compounds (IX). Typical reaction conditions include the use of palladium catalyst, such as Pd₂(dba)₂ or Pd(OAc)₂, a proper ligand as, for example, tri-o-tolylphosphane and a suitable base, as for example K₂CO₃ in a proper solvent, such as N,N-dimethylacetamide or N-methylpyrrolidone, and at an appropriate temperature as 140° C. In some cases, compounds (IX) can be prepared by bromination of compound (VII) with a suitable reagent such as, for example, N-bromosuccinimide, followed by metal-catalyzed cross-coupling reaction of the obtained compounds of formula (X) Typical cross-coupling reaction may be Stille or Suzuki couplings, or similar as described in “Transition Metals for 15 Organic Synthesis”, 2nd Ed, 1, 2004. Representative Suzuki reaction conditions include reacting compound (X) with appropriate boronic acid (XI) or boronic ester (XII), having R₁ as defined above, in the presence of a palladium catalyst, such as Pd(dppf)Cl₂ or Pd(Ph₃P)₄, a suitable base, as for example Na₂CO₃ or K₂CO₃, in a mixture of solvents, such as 1,4-dioxane and water, at an appropriate temperature such as 100° C. Typical Stille cross coupling conditions comprise reacting compound (X) with a suitable organo-tin reagent (XII), wherein R₁ is as described above, in the presence of a palladium catalyst, such as Pd(Ph₃P)₄, a copper source, as for example copper iodide, in a suitable solvent, such as toluene, DMF or 1,4-dioxane, and at an appropriate temperature, as 100° C.

In some cases, wherein R₃ is H, compounds of formula (I) can be obtained by treating compounds (IX) under acidic conditions, using for instance hydrochloridric acid, in a polar protic solvent, as for example ethanol, at an appropriate temperature, such as 60° C.

In another embodiment of the present invention, compounds of formula (I), wherein R₁ and R₂ are defined as above, R₃ is —(C₁-C₆)alkyl, and in particular ethyl, and R₄ is H, can be prepared as described in Scheme 2, using generally known methods.

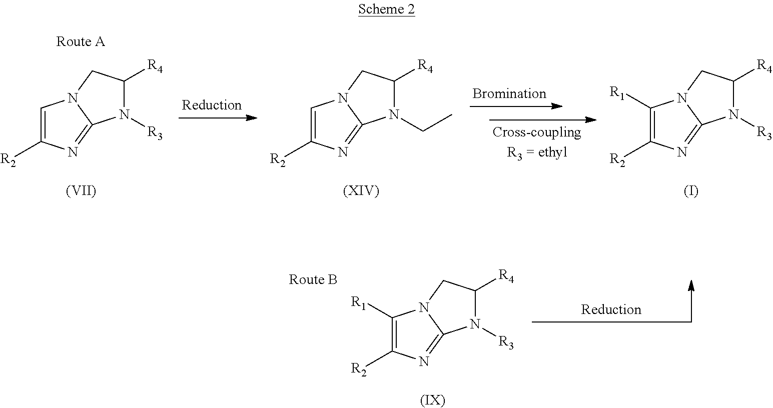

A compound of formula (XIV) can be obtained by reduction treating compounds (VIII) with, for example, borane complexes, such as borane tetrahydrofuran complex or borane dimethylsulfide complex in polar aprotic solvent, such as tetrahydrofuran at appropriate temperature. In some other cases, the reduction may be carried out in the presence of Vaska complex in combination with tetramethyldisiloxane in a suitable solvent, such as DCM and at room temperature (Scheme 2, Route A). Finally, a compound of formula (I) may be obtained from compounds (XIV) by subsequent bromination and cross-coupling reaction following the procedures described above in Scheme 1. Alternatively, in some cases a compound of formula (I), wherein R₁ and R₂ are defined as above, R₃ is —(C₁-C₆)alkyl, and in particular ethyl, and R₄ is H, can be obtained from compounds (IX) by reduction via hydrosilylation using standard conditions to a person skilled in the art (Scheme 2, Route B).

In another embodiment of the present invention, compounds of formula (I) wherein R₁ and R₂ are described as above, R₃ is H and Ra is selected from the groups consisting of —(C₁-C₆)hydroxyalkyl, —C(O)—NR₅R₆, —(C₁-C₆)alkylene-C(O)OH and C(O)OH, may be prepared as reported in Scheme 3.

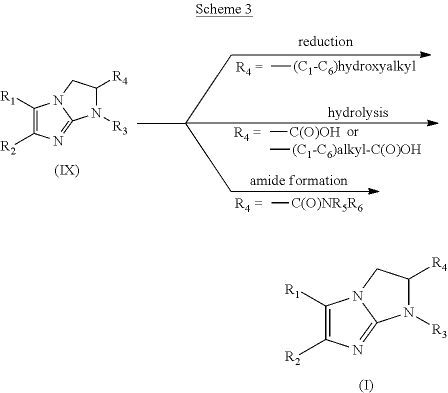

In the cases when R₄ is —(C₁-C₆)hydroxyalkyl, a compound of formula (I) can be obtained from a compound (IX), wherein R₁ and R₂ are described as above, R₃ is —C(O)—(C₁-C₆)alkyl, and R₄ is —C(O)OR₅, by ester group reduction under classical conditions, including for example a proper reductive agent such as NaBH₄ in a suitable solvent or solvent mixture as EtOH/DCM at room temperature.

In some other cases, a compound of formula (I), wherein R₁ and R₂ are described as above, R₃ is —C(O)—(C₁-C₆)alkyl, and R₄ is —C(O)OH or —(C₁-C₆)alkylene-C(O)OH, can be prepared from a compound (IX) by ester hydrolysis under typical acidic conditions using, for example, hydrochloridric acid in a polar solvent, at a proper temperature, such as 60° C.

Finally, in some cases, a compound of formula (I), wherein R₁ and R₂ are described as above, R₃ is H, and R₄ is —C(O) NR₅R₆ may be prepared by treating a compound (IX) having R₄ as —C(O)OR₅ with a suitable amine, such as N-methylamine, in a polar solvent as, for example, tetrahydrofurane. Alternatively, compound (IX) ester hydrolysis, followed by activation of the resulting acid as acyl chloride and amide formation with proper amine under prototypical conditions may lead to compound of formula (I).

### PREPARATIONS OF INTERMEDIATES AND EXAMPLES

Chemical Names of the compounds were generated with ChemDraw Version 19.1.1.21.

All reagents, for which the synthesis is not described in the experimental part, are either commercially available, or are known compounds or may be formed from known compounds by known methods by a person skilled in the art.

### Abbreviation—Meaning

AcOH=acetic acid; Aq.=aqueous; Ar=argon; BBr3=boron tribromide; DCM=dichloromethane; DMA=dimethylacetamide; DMF=dimethylformamide; DMSO=dimethylsulfoxide; ee=enantiomeric excess; Et₂O=diethyl ether; EtOAc=ethyl acetate; EtOH-ethanol, HBr-hydrobromic acid; HCl-hydrochloridric acid; HCOOH-formic acid; HPLC=high performance liquid chromatography; IPA=isopropyl alcohol; K₂CO₃=potassium carbonate; KHCO₃=potassium bicarbonate; KOAc=potassium acetate; LC-MS=liquid chromatography mass spectrometry; LiOH-lithium hydroxide; MeCN-acetonitrile; m-CPBA-meta-chloroperbenzoic acid; N₂=nitrogen; NaBH+=sodium borohydride; NaH=sodium hydride; NaHCO₃=sodium bicarbonate; Na₂SO₃=sodium sulfite; Na₂SO₄=sodium sulfate; NH-silica=amino-functionalized silica; NBS=N-bromo succinimmide; n-BuLi=normal buthyllithium; NMP=N-methyl-2-pyrrolidone; NMR=nuclear magnetic resonance; Pd₂(dba)₃=tris(dibenzylideneacetone)dipalladium(0); Pd(dppf)Cl₂ DCM=[1,1′-bis(diphenylphosphino) ferrocene]dichloropalladium (II), complex with dichloromethane; Pd(OAc)₂=palladium (II) acetate; Pd(PPh₃)₄=tetrakis(triphenylphosphine)palladium(0); pTLC=preparative thin layer chromatography; RP=reverse phase; rt=retention time; RT=room temperature; Sat.=saturated; SCX=strong Cation Exchange; SiO₂=silicon dioxide; SOCl₂=thionyl chloride; TEA=triethylamine; THF=tetrahydrofuran; TMEDA=tetramethylethylenediamine; UPLC-MS=ultra-performance liquid chromatography mass spectrometry

### General Experimental Details and Methods

### Analytical Method

### Instruments, Materials and Methods Employed for Analyses:

### NMR

¹H Nuclear magnetic resonance (NMR) spectroscopy was carried out using a Bruker or Varian instruments operating at 300 or 400 MHz using the stated solvent at around room temperature unless otherwise specified. In all cases, NMR data were consistent with the proposed structures. Characteristic chemical shifts (8) are given in parts-per-million using conventional abbreviations for designation of major peaks: e.g. s, singlet; d, doublet; t, triplet; q, quartet; dd, doublet of doublets; dt, doublet of triplets; m, multiplet; br, broad. Coupling constants (J values) are given in hertz (Hz).

### LC/UV/MS Analytical Methods

Materials: HCCOH≥98% (Sigma-Aldrich), MeCN for HPLC UV/gradient grade (Baker), μQ-water for LCMS.

UPLC-MS measurements were performed on Waters ACQUITY UPLC I-Class PLUS System with Waters SQ Detector 2 (ESI-MS, capillary voltage: 3000 V, cone voltage: 40 V, de-solvation gas: 1000 L/h, de-solvation T: 500° C.), equipped with Acquity UPLC BEH C18 1.7 μm (2.1×100 mm), column no. 186002352, using 20-100% MeCN in water gradient with 0.1% HCOOH (flow: 0.5 mL/min).

### Lcms Methods:

LCMS measurements (for Methods A-D) were performed on Dionex UHPLC Ultimate 3000 with DAD detector/Thermo Scientific MSQ Plus system, equipped with Kinetex® 2.6 μm XB-C18 (4.6×50 mm), 110A, column no. 00B-4496-E0. Detection range: 190-350 nm±4 nm. Flow: 1.0 mL/min. Column temperature: 25° C. Autosampler temperature: 20° C.

LCMS measurements (for Method E) were performed on Aquity UPLC-QDa Mass Spectrometer with a C18-reverse-phase column (50×2.1 mm Acquity CSH with 1.7 μm particle size). Flow: 1.0 mL/min. Column temperature: 40° C. Elution with A: 95/5 water/MeCN+0.05% HCOOH; B: 95/5 MeCN/water+0.05% HCOOH. Detection-MS, UV PDA. MS ionisation method-Electrospray (positive/negative ion).

### Method A:

Analysis time: 7 min. Mobile phase A: 0.1% v/v MQ water solution of HCOOH. Mobile phase B: 0.1% v/v MeCN solution of HCOOH. Elution gradient: 0.00-1.00 min (95% A, 5% B), 4.75-5.25 min (20% A, 80% B), 6.00-7.00 min (95% A, 5% B).

### Method B:

Analysis time: 6 min. Mobile phase A: 0.1% v/v MQ water solution of HCOOH. Mobile phase B: 0.1% v/v MeCN solution of HCOOH. Elution gradient: 0.00 min (90% A, 10% B), 3.35-3.75 min (30% A, 70% B), 3.90-4.75 min (5% A, 95% B), 5.00-6.00 min (90% A, 10% B).

### Method C:

Analysis time: 7 min. Mobile phase A: 0.1% v/v MQ water solution of HCOOH. Mobile phase B: 0.1% v/v MeCN solution of HCOOH. Elution gradient: 0.00 min (100% A, 0% B), 1.00 min (95% A, 5% B), 4.00 min (80% A, 20% B), 4.75-5.25 min (20% A, 80% B), 6.00 min (95% A, 5% B), 7.00 (100% A, 0% B).

### Method D:

Analysis time: 6 min. Mobile phase A: 0.1% v/v MQ water solution of HCOOH. Mobile phase B: 0.1% v/v MeCN solution of HCOOH. Elution gradient: 0.00 min (95% A, 5% B), 3.35-3.75 min (40% A, 60% B), 3.90-4.75 min (5% A, 95% B), 5.00-6.00 min (95% A, 5% B).

### Method E:

Analysis time: 2 min. Mobile phase A: 95/5 water/MeCN+0.05% HCOOH; B: 95/5 MeCN/water+0.05% HCOOH. Elution gradient: 0.00 min (99% A, 1% B), 1.50 min (0.1% A, 99.9% B), 1.90 min (0.1% A, 99.9% B), 2.00 min (99.0% A, 1% B).

### Method F

Analysis time: 2 min. Mobile phase A: 10 mM aqueous solution of NH₄HCO₃ adjusted to pH=10 with ammonia; B: MeCN. Elution gradient: 0.00 min (97% A, 1% B), 1.50 min (0.1% A, 99.9% B), 1.90 min (0.1% A, 99.9% B), 2.00 min (97.0% A, 1% B).

### Preparative HPLC

Purification was performed on a Shimadzu FCV-200AL, coupled with the LC-20AP preparative pump.

### Semi-Preparative Chiral HPLC

Purification was performed on HPLC system Agilent 1100 with an autosampler for injections up to 5 mL and DAD detector. The system is equipped with two G1361A preparative pumps for flow rate up to 100 mL/min and a Gilson FC-203B fraction collector.

### Method F:

Column: Chiralpak AS-H (25×2.0 cm), 5μ; mobile phase: n-Hexane/Ethanol+0.1% isopropylamine 75/25% v/v; flow rate: 17 mL/min; UV detection 220 nm; loop 500 uL; injection 8.2 mg (each injection).

### General Synthetic Procedures

### Procedure A

To a 0.1M solution of the proper Intermediate (1.0 mmol) in DMA or NMP, suitable bromo derivative (1.5 mmol) was added, followed by K₂CO₃ (2.0 eq), tri-o-tolylphosphane (0.1 mmol) and Pd(OAc)₂ (0.05 mmol). The reaction mixture was heated to 140° C. and stirred for 2-16 h. Proper purification yielded the desired product.

### Procedure B

To a solution of the proper Intermediate (1.0 mmol) in a 3:1 mixture of 1,4-dioxane (3.0 mL)/water (1.0 mL), suitable boronic ester or acid (1.2 mmol), K₂CO₃ (3.0 mmol) and Pd(PPh₃)₄ (0.15 mmol) were added. The mixture was heated to 100° C. and stirred for 2-16 h. The reaction was diluted with DCM and quenched with water. The two phases were separated and the organic layer was filtered through a phase separator tube. Volatiles were removed under reduced pressure. Proper purification yielded the desired product.

### Procedure C

To a solution of the proper Intermediate (1.0 mmol) in 1,4 dioxane (0.1M), suitable stannyl derivative (1.0 mmol) was added followed by Pd(PPh₃)₄ (0.05 mmol) and copper (I) iodide (1.0 mmol). The reaction was heated to 100° C. and stirred for 3 h. Then, the reaction was diluted with EtOAc and quenched with water. The two phases were separated and the aqueous layer was extracted with EtOAc. Combined organics were dried over Na₂SO₄, filtered and the solvent removed under reduced pressure. Proper purification yielded the desired product.

### Procedure D

Proper Intermediate (1.0 mmol) was dissolved in EtOH (0.1M) or MeCN (0.1M) and aqueous HCl (5.0 to 10 mmol) was added. The resulting mixture was heated to 60° C. and stirred for 3-12 h. Volatiles were removed under reduced pressure. Proper purification yielded the desired product.

### Preparations of Intermediates

### Intermediate 1:2-(Tributylstannyl)thieno[3,2-c]pyridine

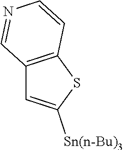

To a solution of thieno[3,2-c]pyridine (1.0 g, 7.41 mmol) and TMEDA (1.22 mL, 8.15 mmol) in dry THF (25 mL), n-BuLi (1.6M in hexanes, 5.1 mL, 8.16 mmol) was added dropwise over 10 minutes at −78° C. under Ar atmosphere. After 10 minutes, tributylchlorostannane (2.21 mL, 8.15 mmol) was added dropwise at −78° C. and the reaction mixture was stirred at this temperature for 1.5 h. Then, reaction was warmed to 0° C. and poured into water. The resulting suspension was extracted with EtOAc. Combined organics were dried over Na₂SO₄, filtered and solvent removed under reduced pressure. The residue was dissolved in hexane, filtered and dried to yield the title compound (2.77 g, 6.51 mmol, 88% yield) as a light brown oil.

UPLC-MS: m/z (M-1) 456 (MeOH adduct, tin isotopic pattern), rt=1.46 min.

### Intermediate 2:6-(4,4,5,5-Tetramethyl-1,3,2-dioxaborolan-2-yl)-[1,2,4]triazolo[1,5-a]pyridine

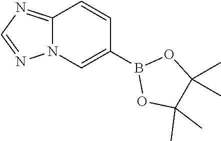 [SMILES: CC1(C)OB(c2ccc3ncnn3c2)OC1(C)C]

To an Ar-saturated suspension of 6-bromo-[1,2,4]triazolo[1,5-a]pyridine (25.0 g, 126.9 mmol), bis(pinacolato)diboron (38.68 g, 152.3 mmol) and KOAc (37.37 g, 380.8 mmol) in DMF (20 mL), PdCl₂(dppf)·DCM (10.37 g, 12.7 mmol) was added. The reaction mixture was stirred at 100° C. for 3 h. After cooling to RT, DMF was evaporated under reduced pressure. Water was added to the residue and the suspension was extracted with EtOAc. Combined organics were dried over Na₂SO₄, filtered and evaporated under reduced pressure. The residue was triturated with hexane. The suspension was filtered and the filtrate evaporated to give the title compound (19.5 g, 79.5 mmol, 63% yield) as colorless oil which crystallized upon cooling to RT.

UPLC-MS: m/z (M+1) 162, rt=1.19 min (boronic acid).

### Intermediate 3:4,6-Dichloro-2H-pyran-2-one

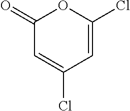

Under N₂ atmosphere, phosporus chloride (1.2 g, 5.74 mmol) was added portionwise to dimethyl 3-oxopentanedioate (1 g, 5.74 mmol) at 0° C. The reaction was heated to 50° C. for 1 h, then cooled to RT, diluted with DCM and quenched with water. The two phases were separated and the aqueous layer was extracted with DCM. Combined organics were filtered through a phase separator tube and the solvent was removed under reduced pressure to give a red oil. Aqueous hydrochloric acid (20%, 6 mL) was added, and the mixture was refluxed for 1.5 h. Volatiles were evaporated and the residue was dissolved in Et₂O (50 mL). The resulting solution was dried over calcium chloride, filtered, and solvent removed under reduced pressure. The isolated (Z)-3-chloropent-2-enedioic acid (945 mg, 5.74 mmol) was added portionwise at 0° C. to phosphorus chloride (2.39 g, 11.49 mmol). The mixture was maintained at 0° C. until solids liquefied. The resulting red solution was stirred at RT for 1 h, heated to 100° C. for 15 min, and then cooled to RT. The mixture was diluted with DCM and extracted with water. Combined organics were filtered through a Celite® pad, and the dark red solution was neutralized to pH 7 by slow addition of sat. aqueous NaHCO₃. The organic phase was separated through a phase separator tube, and the solvent was removed under reduced pressure. Purification by flash chromatography (isocratic elution with 100% DCM) yielded the title compound (295 mg, 1.788 mmol, 31% yield) as dark yellow oil.

LC-MS (ESI): m/z (M+1) no ionization, rt=0.74 min (Method E).

### Intermediate 4:4-Chloro-2H-pyran-2-one

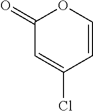

To a solution of Intermediate 3 (290 mg, 1.758 mmol) in acetic acid (2 mL), zinc powder (138 mg, 2.109 mmol) was added. The reaction mixture was stirred at RT for 16 h. The solid was filtered, and acetic acid was removed by evaporation. The residue was taken up in DCM/water (15:5 mL) and neutralized to pH 7 by addition of solid KHCO₃. The two phases were separated and the aqueous layer extracted with DCM. Combined organics were filtered through a phase separator tube, and the solvent was removed under reduced pressure. Purification by flash chromatography (isocratic elution with 100% DCM) yielded the title compound (41 mg, 0.314 mmol, 18% yield) as off-white crystals.

LC-MS (ESI): m/z (M+1) 130.8-132.8, rt=0.47 min (Method E).

### Intermediate 5:4-(Trimethylstannyl)-2H-pyran-2-one

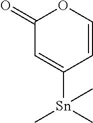

To a solution of Intermediate 4 (74 mg, 0.567 mmol) in THF (2 mL), 1,1,1,2,2,2-hexamethyldistannane (0.141 mL, 0.680 mmol) and Pd(PPh₃)+ (0.020 mg, 0.017 mmol) were added. The mixture was heated to reflux under Ar for 44 h, then cooled to RT. The solvent was removed under reduced pressure. Purification by flash chromatography (gradient of elution from 0% to 100% of EtOAc in heptane) yielded the title compound (80 mg, 0.309 mmol, 54% yield) as off-white solid.

LC-MS (ESI): m/z (M+1) 261.1, rt=0.96 min (tin isotopic pattern, Method E).

### Intermediate 6:5-Bromo-2H-pyran-2-one

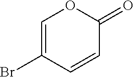

To a solution of 2H-pyran-2-one (0.120 mL, 1.488 mmol) in dry DCM (1.0 mL) cooled to −78° C., a solution of bromine (0.092 mL, 1.786 mmol) in DCM (0.5 mL) was added dropwise while shining light (hv=160-467 nm) on the reaction vessel. The reaction was stirred at the same temperature for 5 h. Volatiles were removed under reduced pressure to give the crude 5,6-dibromo-5,6-dihydro-2H-pyran-2-one as an orange oil. The crude was taken up in DCM (1.0 mL) and TEA (0.207 mL, 1.488 mmol) was added at 0° C. The reaction was stirred for 30 min. Volatiles were removed under reduced pressure and the crude was suspended in Et₂O, filtered and the filtrate evaporated under reduced pressure. Purification by flash chromatography (isocratic elution with DCM 100%) yielded the title compound (193.6 mg, 1.106 mmol, 74% yield) as a white solid.

LC-MS (ESI): m/z (M+1) 175.1, rt=0.50 min (Method E).

### Intermediate 7:5-(Trimethylstannyl)-2H-pyran-2-one

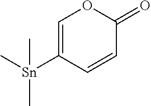

Prepared from Intermediate 6 (100 mg, 0.571 mmol) following similar procedure as for Intermediate 5. Purification by silica gel flash chromatography (gradient of elution from 0% to 50% of EtOAc in heptane) yielded the title compound (104 mg, 0.400 mmol, 70% yield) as a pale brown oil.

LC-MS (ESI): m/z (M+1) 261.0, rt=0.97 min (Method E).

### Intermediate 8:2-Bromothieno[3,2-c]pyridine

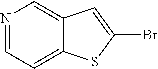

To a solution of thieno[3,2-c]pyridine (200 mg, 1.479 mmol) in dry THF (2 mL), cooled to −78° C., n-BuLi 2.5 M in hexane (0.651 mL, 1.627 mmol) was added and the reaction stirred for 30 min. Then, bromine (0.083 mL, 1.627 mmol) was added and the reaction allowed to warm to RT and stirred for 12 h. The reaction was diluted with EtOAc and quenched with water. The two phases were separated and the aqueous layer extracted with EtOAc. Combined organics were dried over Na₂SO₄, filtered and the solvent evaporated under reduced pressure. Purification by flash chromatography (gradient of elution from 5% to 100% of EtOAc in heptane) yielded the title compound (0.073 g, 0.341 mmol, 23% yield) as beige powder.

LC-MS (ESI): m/z (M+1) 213.8-215.8, rt=0.39 min (Method E).

### Intermediate 9:2-Bromo-1-(6-methylpyridin-2-yl) ethan-1-one

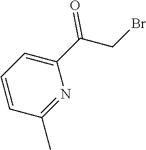

1-(6-Methylpyridin-2-yl) ethan-1-one (50 g, 370 mmol) was dissolved in HBr solution in AcOH 33% (100 mL). Then, bromine (21 mL, 407.4 mmol) was added dropwise to the solution over 30 minutes. The reaction was stirred for 6 h at RT, then diluted with DCM (500 mL) and filtered. The dried solid was suspended in DCM/water mixture (1:1) (1.5 L) and neutralized with solid NaHCO₃. The two phases were separated and the aqueous layer extracted with DCM. Combined organics were dried over Na₂SO₄, filtered and volatiles were removed under reduced pressure to yield the title compound (36.7 g, 172.0 mmol, 46% yield) as colorless oil which crystallized upon cooling.

UPLC-MS (254 nm): m/z (M+1) 214, rt=1.68 min.

### Intermediate 10:2-Bromo-1-(5-chloro-2-fluorophenyl) ethan-1-one

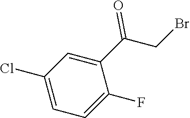 [SMILES: O=C(CBr)c1cc(Cl)ccc1F]

Prepared from 1-(5-chloro-2-fluorophenyl) ethan-1-one (500 mg, 2.90 mmol) following a similar procedure as for Intermediate 9. Purification by flash chromatography (gradient of elution from 0% to 50% of EtOAc in heptane) yielded the title compound (546 mg, 2.17 mmol, 75% yield) as white crystal solid.

LC-MS (ESI): m/z (M+1) no ionization, rt=1.09 min (Method E).

### Intermediate 11:2-Bromo-1-(4-methylthiazol-2-yl) ethan-1-one

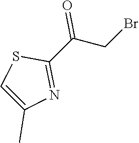

Prepared from 1-(4-methylthiazol-2-yl) ethan-1-one (2.0 g, 14.17 mmol), following similar procedure as for Intermediate 9. Purification by flash chromatography (gradient of elution from 0% to 100% of DCM in heptane) yielded the title compound (2.7 g, 12.27 mmol, 87% yield) as red crystal solid.

LC-MS (ESI): m/z (M+1) 221.9, rt=0.86 min (Method E).

### Intermediate 12:2-Bromo-1-(1-methyl-1H-pyrazol-3-yl) ethan-1-one

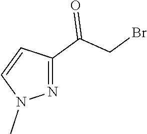

Prepared from 1-(1-methyl-1H-pyrazol-3-yl) ethan-1-one (500 mg, 4.03 mmol), following similar procedure as for Intermediate 9. Purification by NH-silica flash chromatography (gradient of elution from 0% to 100% of EtOAc in heptane) yielded the title compound (240 mg, 1.182 mmol, 29% yield) as yellow solid.

LC-MS (ESI): m/z (M+1) 204.9, rt=0.61 min (Method E).

### Intermediate 13:2-Bromo-1-(6-chloropyridin-2-yl) ethan-1-one

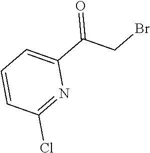 [SMILES: O=C(CBr)c1cccc(Cl)n1]

Prepared from 1-(6-chloropyridin-2-yl) ethan-1-one (1 g, 6.430 mmol), following similar procedure as for Intermediate 9. Purification by flash chromatography (gradient of elution from 0% to 100% of DCM in heptane) yielded the title compound (1.20 g, 5.12 mmol, 80% yield) as red solid.

LC-MS (ESI): m/z (M+1) 235.8, rt=0.97 min (Method E).

### Intermediate 14: N-(4-(6-Methylpyridin-2-yl)-1H-imidazol-2-yl) acetamide

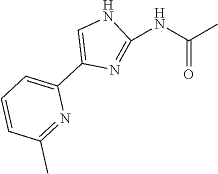 [SMILES: CC(=O)Nc1nc(-c2cccc(C)n2)c[nH]1]

To a suspension of 2-bromo-1-(6-methylpyridin-2-yl) ethan-1-one (Intermediate 9, 50.4 g, 253.3 mmol) in MeCN (50 mL), N-acetylguanidine (30.7 g, 304.0 mmol) and K₂CO₃ (42.0 g, 304.0 mmol) were added. The reaction mixture was stirred at 50° C. for 16 h. Volatiles were removed under reduced pressure, the residue was triturated with MeCN and washed with water to yield the title compound (13.2 g, 61.1 mmol, 24% yield) as brown solid.

UPLC-MS (254 nm): m/z (M+1) 217, rt=1.20 min.

### Intermediate 15: N-(5-(5-Chloro-2-fluorophenyl)-1H-imidazol-2-yl) acetamide

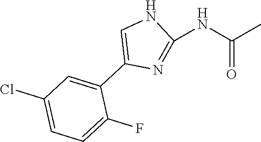 [SMILES: CC(=O)Nc1nc(-c2cc(Cl)ccc2F)c[nH]1]

Prepared from Intermediate 10 (1 g, 3.98 mmol) following similar procedure as for Intermediate 14. Purification by flash chromatography (gradient of elution from 0% to 100% of EtOAc in heptane) yielded the title compound (418 mg, 1.648 mmol, 41% yield) as pale yellow solid.

LC-MS (ESI): m/z (M+1) 254.0, rt=0.76 min (Method E).

### Intermediate 16: N-(5-(4-Methylthiazol-2-yl)-1H-imidazol-2-yl) acetamide

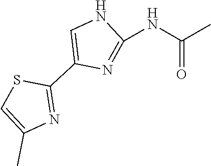

Prepared from Intermediate 11 (2.9 g, 13.18 mmol), following similar procedure as for Intermediate 14. Purification by RP flash chromatography (gradient of elution from 0% to 100% of B in A; A: water/MeCN 95:5+0.1% HCOOH, B: MeCN/water 95:5+0.1% HCOOH) yielded the title compound (545 mg, 2.452 mmol, 19% yield) as an orange solid.

LC-MS (ESI): m/z (M+1) 223.0, rt=0.46 min (Method E).

### Intermediate 17: N-(5-(1-Methyl-1H-pyrazol-3-yl)-1H-imidazol-2-yl) acetamide

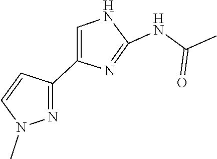

Prepared from Intermediate 12 (1.7 g, 8.37 mmol), following similar procedure as for Intermediate 14. Purification by RP flash chromatography (gradient of elution from 0% to 100% of B in A; A: water/MeCN 95:5+0.1% HCOOH, B: MeCN/water 95:5+0.1% HCOOH) yielded the title compound (600 mg, 2.92 mmol, 35% yield) as off-white solid.

LC-MS (ESI): m/z (M+1) 205.9, rt=0.26 min (Method E).

### Intermediate 18: N-(5-(6-Chloropyridin-2-yl)-1H-imidazol-2-yl) acetamide

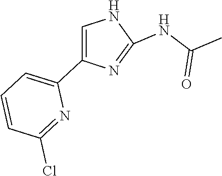 [SMILES: CC(=O)Nc1nc(-c2cccc(Cl)n2)c[nH]1]

Prepared from Intermediate 13 (3.65 g, 15.57 mmol), following similar procedure as for Intermediate 14. Purification by flash chromatography (gradient of elution from 0% to 100% of EtOAc in heptane) yielded the title compound (500 mg, 2.113 mmol, 14% yield) as an off-white solid.

LC-MS (ESI): m/z (M+1) 237.0-238.9, rt=0.52 min (Method E).

### Intermediate 19:1-(6-(6-Methylpyridin-2-yl)-2,3-dihydro-1H-imidazo[1,2-a]imidazol-1-yl) ethan-1-one

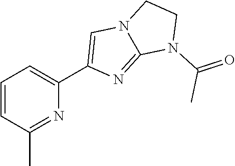 [SMILES: CC(=O)N1CCn2cc(-c3cccc(C)n3)nc21]

To a suspension of Intermediate 14 (13.2 g, 61.1 mmol) in MeCN (50 mL), K₂CO₃ (42.2 g, 305.4 mmol) was added followed by dropwise addition of a solution of 1,2-dibromoethane (16.0 mL, 183.3 mmol) in MeCN (30 mL). The reaction was heated to 80° C. and stirred for 16 h. Solvent was evaporated, the residue was dissolved in a DCM/MeOH mixture (4:1 vol/vol) and filtered through silica. Volatiles were removed under reduced pressure. The residue was crystallized from EtOAc to yield the title compound (7.5 g, 31.0 mmol, 51% yield) (major regioisomer) as beige powder.

UPLC-MS: m/z (M+1) 243.0, rt=1.20 min.

### Intermediate 20:1-(5-Bromo-6-(6-methylpyridin-2-yl)-2,3-dihydro-1H-imidazo[1,2-a]imidazol-1-yl) ethan-1-one

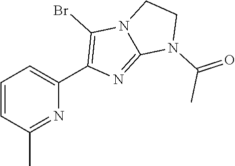 [SMILES: CC(=O)N1CCn2c1nc(-c1cccc(C)n1)c2Br]

NBS (7.4 g, 41.5 mmol) was added portionwise to a stirred solution of Intermediate 19 (10.0 g, 41.3 mmol) in dry DCM (15 mL), cooled at 0° C. The mixture was stirred for 30 minutes, then was diluted with water and extracted DCM. Combined organics were dried over Na₂SO₄, filtered and volatiles removed under reduced pressure to give the title compound (12.9 g, 40.3 mmol, 98% yield) as beige powder, then used in further steps without purification.

UPLC-MS: m/z (M+1) 320.9-322.9, rt=1.23 min.

### Intermediate 21:1-(6-(5-Chloro-2-fluorophenyl)-2,3-dihydro-1H-imidazo[1,2-a]imidazol-1-yl) ethan-1-one

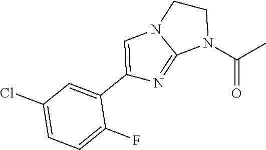 [SMILES: CC(=O)N1CCn2cc(-c3cc(Cl)ccc3F)nc21]

Prepared from Intermediate 15 (488 mg, 1.924 mmol) following similar procedure as for Intermediate 19. Purification by RP flash chromatography (gradient of elution from 0% to 100% of B in A; A: water/MeCN 95:5+0.1% HCOOH, B: MeCN/water 95:5+0.1% HCOOH) yielded the title compound (187 mg, 0.669 mmol, 35% yield) (major regioisomer) as yellow solid.

LC-MS (ESI): m/z (M+1) 280.0, rt=0.73 min (Method E).

### Intermediate 22:1-(5-Bromo-6-(5-chloro-2-fluorophenyl)-2,3-dihydro-1H-imidazo[1,2-a]imidazol-1-yl) ethan-1-one

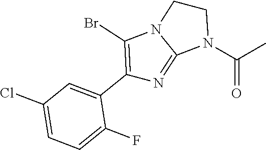 [SMILES: CC(=O)N1CCn2c1nc(-c1cc(Cl)ccc1F)c2Br]

Prepared from Intermediate 21 (187 mg, 0.669 mmol) following the procedure used for the Intermediate 20. The crude title compound (assumed quantitative yield) was used in the next step without purification.

LC-MS (ESI): m/z (M+1) 359.9-361.0, rt=1.09 min (Method E).

### Intermediate 23:1-(6-(4-Methylthiazol-2-yl)-2,3-dihydro-1H-imidazo[1,2-a]imidazol-1-yl) ethan-1-one

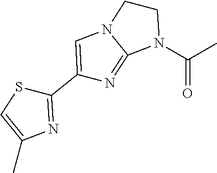 [SMILES: CC(=O)N1CCn2cc(-c3nc(C)cs3)nc21]

Prepared from Intermediate 16 (540 mg, 2.430 mmol), following similar procedure as for Intermediate 19. Purification by RP flash chromatography (gradient of elution from 0% to 100% of B in A; A: water/MeCN 95:5+0.1% HCOOH, B: MeCN/water 95:5+0.1% HCOOH) yielded the title compound (290 mg, 1.168 mmol, 48% yield) (single observed regioisomer) as an orange solid.

LC-MS (ESI): m/z (M+1) 249.0, rt=0.58 min (Method E).

### Intermediate 24:1-(5-Bromo-6-(4-methylthiazol-2-yl)-2,3-dihydro-1H-imidazo[1,2-a]imidazol-1-yl) ethan-1-one

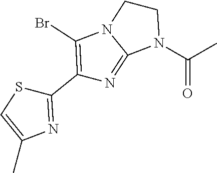 [SMILES: CC(=O)N1CCn2c1nc(-c1nc(C)cs1)c2Br]

Prepared from Intermediate 23 (237 mg, 0.954 mmol) following similar procedure as for Intermediate 20. Purification by RP flash chromatography (gradient of elution from 0% to 100% of B in A; A: water/MeCN 95:5+0.1% HCOOH, B: MeCN/water 95:5+0.1% HCOOH) yielded the title compound (177 mg, 0.541 mmol, 57% yield) as off-white solid.

LC-MS (ESI): m/z (M+1) 327.0-329.0, rt=0.75 min (Method E).

### Intermediate 25:1-(6-(1-Methyl-1H-pyrazol-3-yl)-2,3-dihydro-1H-imidazo[1,2-a]imidazol-1-yl) ethan-1-one

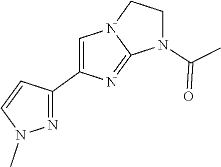 [SMILES: CC(=O)N1CCn2cc(-c3ccn(C)n3)nc21]

Prepared from Intermediate 17 (540 mg, 2.63 mmol), following similar procedure as for Intermediate 19. Purification by RP flash chromatography (gradient of elution from 0% to 100% of B in A; A: water/MeCN 95:5+0.1% HCOOH, B: MeCN/water 95:5+0.1% HCOOH) yielded the title compound (225 mg, 0.973 mmol, 37.0% yield) (major regioisomer) as white solid.

LC-MS (ESI): m/z (M+1) 232.0, rt=0.31 min (Method E).

### Intermediate 26:1-(6-(6-Chloropyridin-2-yl)-2,3-dihydro-1H-imidazo[1,2-a]imidazol-1-yl) ethan-1-one

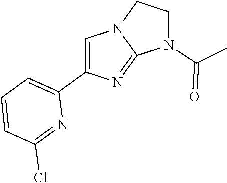 [SMILES: CC(=O)N1CCn2cc(-c3cccc(Cl)n3)nc21]

Prepared from Intermediate 18 (420 mg, 1.775 mmol), following similar procedure as for Intermediate 19. Purification by RP flash chromatography (gradient of elution from 0% to 100% of B in A; A: water/MeCN 95:5+0.1% HCOOH, B: MeCN/water 95:5+0.1% HCOOH) yielded the title compound (365 mg, 1.389 mmol, 78% yield) (major regioisomer) as off-white solid.

LC-MS (ESI): m/z (M+1) 263.0, rt=0.70 min (Method E).

### Intermediate 27: Ethyl 1-acetyl-6-(6-methylpyridin-2-yl)-2,3-dihydro-1H-imidazo[1,2-a]imidazole-2-carboxylate

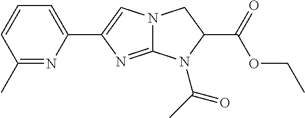 [SMILES: CCOC(=O)C1Cn2cc(-c3cccc(C)n3)nc2N1C(C)=O]

Prepared from Intermediate 14 (1.0 g, 4.63 mmol) and ethyl 2,3-dibromopropanoate (3.36 mL, 23.14 mmol) following a similar procedure as for Intermediate 19. Purification by flash chromatography (isocratic elution with hexane/EtOAc/Et₃N 1:5:0.01) yielded the title compound (0.72 g, 2.29 mmol, 50% yield) as a white solid.

UPLC-MS: m/z (M+1) 315.0, rt=1.26 min.

### Intermediate 28: Ethyl 1-acetyl-5-bromo-6-(6-methylpyridin-2-yl)-2,3-dihydro-1H-imidazo[1,2-a]imidazole-2-carboxylate

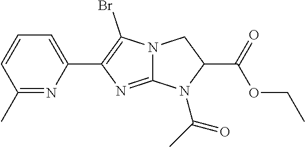 [SMILES: CCOC(=O)C1Cn2c(nc(-c3cccc(C)n3)c2Br)N1C(C)=O]

Prepared from Intermediate 27 (500 mg, 1.59 mmol) following a similar procedure as for Intermediate 20. Title compound (510 mg, 1.30 mmol, 82% yield) was obtained as pale yellow powder.

UPLC-MS: m/z (M+1) 393.0, rt=1.33 min.

### Intermediate 29: Ethyl 2-(1-acetyl-6-(6-methylpyridin-2-yl)-2,3-dihydro-1H-imidazo[1,2-a]imidazol-2-yl)acetate

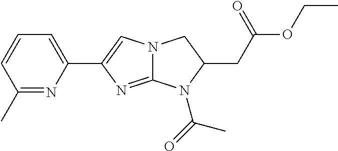 [SMILES: CCOC(=O)CC1Cn2cc(-c3cccc(C)n3)nc2N1C(C)=O]

Prepared from Intermediate 14 (1.0 g, 4.63 mmol) and ethyl 3,4-dibromobutanoate (1.9 g, 6.94 mmol) following similar procedure as for Intermediate 19. Purification by flash chromatography (isocratic elution with hexane/EtOAc/Et₃N 1:5:0.01) yielded the title compound (683 mg, 2.08 mmol, 45% yield) was obtained as yellow powder.

UPLC-MS: m/z (M+1) 330.0, rt=1.27 min.

### Intermediate 30: Ethyl 2-(1-acetyl-5-bromo-6-(6-methylpyridin-2-yl)-2,3-dihydro-1H-imidazo[1,2-a]imidazol-2-yl)acetate

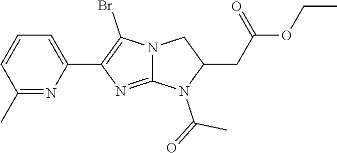 [SMILES: CCOC(=O)CC1Cn2c(nc(-c3cccc(C)n3)c2Br)N1C(C)=O]

Prepared from Intermediate 29 (500 mg, 1.52 mmol) following similar procedure as for Intermediate 20. The title compound (559 mg, 1.38 mmol, 90% yield) was obtained as an off-white powder.

UPLC-MS: m/z (M+1) 408.0, rt=1.32 min.

### Intermediate 31: Methyl 2-bromo-5-(methylthio)benzoate

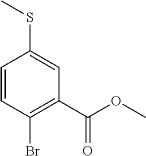

To a solution of methyl 2-bromo-5-fluorobenzoate (1 g, 4.29 mmol) in dry DMF (10 mL), sodium methanethiolate (0.271 g, 3.86 mmol) was added and the reaction was stirred at RT for 6 h. Then, solvent was evaporated and the residue was dissolved in DCM (50 mL) and washed with brine. Organic phase was filtered through a phase separator tube, and solvent was removed under reduced pressure. Purification by flash chromatography (gradient of elution from 0 to 5% of EtOAc in heptane) yielded the title compound (546 mg, 2.091 mmol, 49% yield) as colorless oil.

LC-MS (ESI): m/z (M+1) 261.1-263.0, rt=1.14 min (Method E).

### Intermediate 32:1-(5-([1,2,4]Triazolo[1,5-a]pyridin-6-yl)-6-(5-chloro-2-fluorophenyl)-2,3-dihydro-1H-imidazo[1,2-a]imidazol-1-yl) ethan-1-one

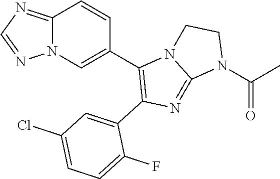 [SMILES: CC(=O)N1CCn2c1nc(-c1cc(Cl)ccc1F)c2-c1ccc2ncnn2c1]

Prepared from Intermediate 21 (250 mg, 0.894 mmol) and 6-bromo-[1,2,4]triazolo[1,5-a]pyridine (265 mg, 1.341 mmol) following procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (115 mg, 0.290 mmol, 32% yield) as yellow solid.

LC-MS (ESI): m/z (M+1) 398.0, rt=1.03 min (Method E).

### Intermediate 33:1-(5-([1,2,4]Triazolo[1,5-a]pyridin-6-yl)-6-(4-methylthiazol-2-yl)-2,3-dihydro-1H-imidazo[1,2-a]imidazol-1-yl) ethan-1-one

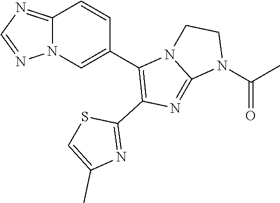 [SMILES: CC(=O)N1CCn2c1nc(-c1nc(C)cs1)c2-c1ccc2ncnn2c1]

Prepared from Intermediate 24 (100 mg, 0.306 mmol) and [1,2,4]triazolo[1,5-a]pyridin-6-ylboronic acid (Intermediate 2, 60 mg, 0.367 mmol) following procedure B. Purification by RP flash chromatography (gradient of elution from 0% to 100% of B in A in 16 CV, A: water/MeCN 95:5+0.1% HCOOH, B: MeCN/water 95:5+0.1% HCOOH) yielded the title compound (13.5 mg, 0.037 mmol, 12% yield) as yellow solid.

LC-MS (ESI): m/z (M+1) 366.0, rt=0.72 min (Method E)

### Intermediate 34:4-(1-Acetyl-6-(6-methylpyridin-2-yl)-2,3-dihydro-1H-imidazo[1,2-a]imidazol-5-yl)-2H-pyran-2-one

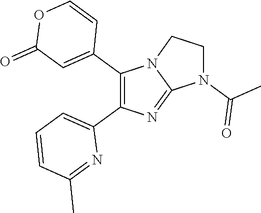 [SMILES: CC(=O)N1CCn2c1nc(-c1cccc(C)n1)c2-c1ccoc(=O)c1]

Prepared from Intermediate 20 (100 mg, 0.311 mmol) and Intermediate 5 (81 mg, 0.311 mmol) following procedure C. Purification by RP flash chromatography (gradient of elution from 0% to 100% of B in A, A: water/MeCN 95:5+0.1% HCOOH, B: MeCN/water 95:5+0.1% HCOOH) yielded the title compound (105 mg, 0.312 mmol, 100% yield) as white solid.

LC-MS (ESI): m/z (M+1) 337.3, rt=0.43 min (Method E).

### Intermediate 35:5-(1-acetyl-6-(6-methylpyridin-2-yl)-2,3-dihydro-1H-imidazo[1,2-a]imidazol-5-yl)-2H-pyran-2-one

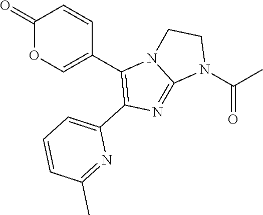 [SMILES: CC(=O)N1CCn2c1nc(-c1cccc(C)n1)c2-c1ccc(=O)oc1]

Prepared from Intermediate 20 (108 mg, 0.335 mmol) and Intermediate 7 (104 mg, 0.402 mmol) following procedure C. Purification by NH-silica flash chromatography (gradient of elution from 0 to 100% of DCM/MeOH 9:1 in DCM) yielded the title compound (15 mg, 0.045 mmol, 13% yield) as a yellow solid.

LC-MS (ESI): m/z (M+1) 337.3, rt=0.33 min (Method E).

### Intermediate 36: Ethyl 1-acetyl-6-(6-methylpyridin-2-yl)-5-(thieno[3,2-c]pyridin-2-yl)-2,3-dihydro-1H-imidazo[1,2-a]imidazole-2-carboxylate

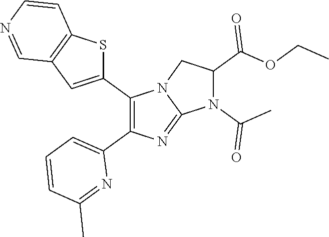 [SMILES: CCOC(=O)C1Cn2c(nc(-c3cccc(C)n3)c2-c2cc3cnccc3s2)N1C(C)=O]

Prepared from Intermediate 28 (480 mg, 1.22 mmol) and Intermediate 1 (781 mg, 1.84 mmol) following Procedure C. The crude was triturated from hexanes and purified by flash chromatography (isocratic elution with hexanes/EtOAc/Et₃N 1/5/0.01) to yield the title compound (160 mg, 0.36 mmol, 29% yield) as yellow crystalline solid.

UPLC-MS: m/z (M+1) 449, rt=1.31 min.

### Intermediate 37:1-(6-(4-Methylthiazol-2-yl)-5-(thieno[3,2-c]pyridin-2-yl)-2,3-dihydro-1H-imidazo[1,2-a]imidazol-1-yl) ethan-1-one

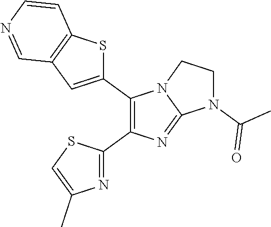 [SMILES: CC(=O)N1CCn2c1nc(-c1nc(C)cs1)c2-c1cc2cnccc2s1]

Prepared from Intermediate 24 (180 mg, 0.550 mmol) and Intermediate (350 mg, 0.825 mmol) following procedure C. Purification by RP flash chromatography (gradient of elution from 0% to 100% of B in A; A: water/MeCN 95:5+0.1% HCOOH, B: MeCN/water 95:5+0.1% HCOOH) yielded the title compound (16.6 mg, 0.044 mmol, 8% yield) as yellow solid.

LC-MS (ESI): m/z (M+1) 381.9, rt=0.60 min (Method E).

### Intermediate 38:1-(5-([1,2,4]Triazolo[1,5-a]pyridin-6-yl)-6-(1-methyl-1H-pyrazol-3-yl)-2,3-dihydro-1H-imidazo[1,2-a]imidazol-1-yl) ethan-1-one

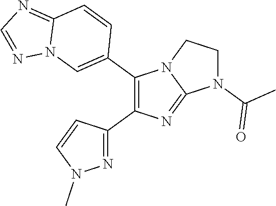 [SMILES: CC(=O)N1CCn2c1nc(-c1ccn(C)n1)c2-c1ccc2ncnn2c1]

Prepared from Intermediate 25 (100 mg, 0.432 mmol) and 6-bromo-[1,2,4]triazolo[1,5-a]pyridine (128 mg, 0.649 mmol) following Procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (123 mg, 0.353 mmol, 82% yield) as yellow solid.

LC-MS (ESI): m/z (M+1) 349.0, rt=0.49 min (Method E).

### Intermediate 39:1-(6-(1-Methyl-1H-pyrazol-3-yl)-5-(thieno[3,2-c]pyridin-2-yl)-2,3-dihydro-1H-imidazo[1,2-a]imidazol-1-yl) ethan-1-one

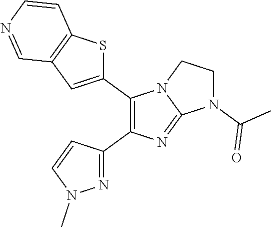 [SMILES: CC(=O)N1CCn2c1nc(-c1ccn(C)n1)c2-c1cc2cnccc2s1]

Prepared from Intermediate 25 (120 mg, 0.519 mmol) and Intermediate 8 (167 mg, 0.778 mmol) following Procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (60 mg, 0.165 mmol, 32% yield) as yellow solid.

LC-MS (ESI): m/z (M+1) 365.0, rt=0.41 min (Method E).

### Intermediate 40:1-(5-([1,2,4]Triazolo[1,5-a]pyridin-6-yl)-6-(6-chloropyridin-2-yl)-2,3-dihydro-1H-imidazo[1,2-a]imidazol-1-yl) ethan-1-one

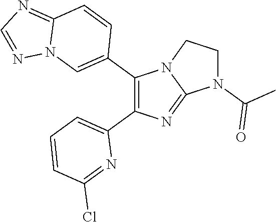 [SMILES: CC(=O)N1CCn2c1nc(-c1cccc(Cl)n1)c2-c1ccc2ncnn2c1]

Prepared from Intermediate 26 (150 mg, 0.571 mmol) and 6-bromo-[1,2,4]triazolo[1,5-a]pyridine (170 mg, 0.857 mmol) following Procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (175 mg, 0.461 mmol, 81% yield) as yellow solid.

LC-MS (ESI): m/z (M+1) 379.9, rt=0.79 min (Method E).

### Intermediate 41:1-(6-(6-Chloropyridin-2-yl)-5-(thieno[3,2-c]pyridin-2-yl)-2,3-dihydro-1H-imidazo[1,2-a]imidazol-1-yl) ethan-1-one

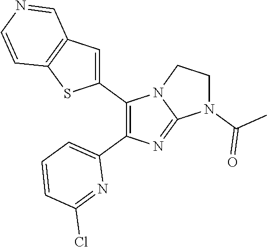 [SMILES: CC(=O)N1CCn2c1nc(-c1cccc(Cl)n1)c2-c1cc2cnccc2s1]

Prepared from Intermediate 26 (90 mg, 0.343 mmol) and Intermediate 8 (110 mg, 0.514 mmol) following Procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (50 mg, 0.126 mmol, 37% yield) as yellow solid.

LC-MS (ESI): m/z (M+1) 395.9, rt=0.63 min (Method E).

### Intermediate 42:1-(6-(5-Chloro-2-fluorophenyl)-5-(thieno[3,2-c]pyridin-2-yl)-2,3-dihydro-1H-imidazo[1,2-a]imidazol-1-yl) ethan-1-one

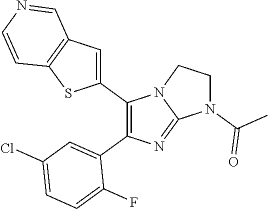 [SMILES: CC(=O)N1CCn2c1nc(-c1cc(Cl)ccc1F)c2-c1cc2cnccc2s1]

Prepared from Intermediate 22 (316 mg, 0.881 mmol) and Intermediate 1 (561 mg, 1.322 mmol) following procedure C. Purification by RP flash chromatography (gradient of elution from 0% to 100% of B in A; A: water/MeCN 95:5+0.1% HCOOH, B: MeCN/water 95:5+0.1% HCOOH) yielded the title compound (188 mg, 0.455 mmol, 52% yield) as yellow solid.

LC-MS (ESI): m/z (M+1) 413.0, rt=0.67 min (Method E).

### Intermediate 43:1-Ethyl-6-(6-methylpyridin-2-yl)-2,3-dihydro-1H-imidazo[1,2-a]imidazole

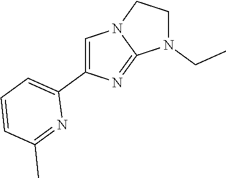 [SMILES: CCN1CCn2cc(-c3cccc(C)n3)nc21]

To a solution of Intermediate 19 (90 mg, 0.373 mmol) in THF (2 mL), borane-tetrahydrofuran complex 1.0M solution in THF (1.119 mL, 1.119 mmol) was added and the reaction heated to 50° C. and stirred for 3 h. The solvent was removed under reduced pressure. Purification by RP flash chromatography (gradient of elution from 0% to 100% of B in A; A: water/MeCN 95:5+0.1% HCOOH, B: MeCN/water 95:5+0.1% HCOOH) yielded the title compound (50 mg, 0.219 mmol, 59% yield) as yellowish solid.

LC-MS (ESI): m/z (M+1) 229.0, rt=0.35 min (Method E).

### Intermediate 44:1-(5-(Benzo[c][1,2,5] thiadiazol-4-yl)-6-(6-methylpyridin-2-yl)-2,3-dihydro-1H-imidazo[1,2-a]imidazol-1-yl) ethanone

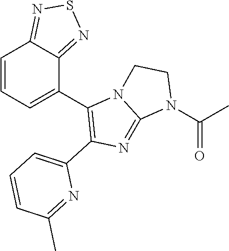 [SMILES: CC(=O)N1CCn2c1nc(-c1cccc(C)n1)c2-c1cccc2nsnc12]

Prepared from Intermediate 19 (50 mg, 0.206 mmol) and 4-bromobenzo[c][1,2,5] thiadiazole (66.6 mg, 0.310 mmol) following Procedure A. Purification by NH-silica flash chromatography (gradient of elution from 0 to 100% of EtOAc in heptane) yielded the title compound (40 mg, 0.106 mmol, 51% yield) as yellow solid.

LC-MS (ESI): m/z (M+1) 377.4, rt=0.47 min (Method E)

### Intermediate 45:1-(5-(Benzo[d][1,2,3] thiadiazol-7-yl)-6-(6-methylpyridin-2-yl)-2,3-dihydro-1H-imidazo[1,2-a]imidazol-1-yl) ethan-1-one

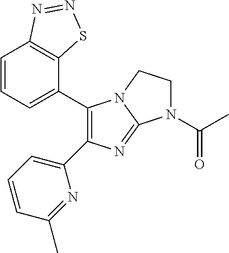 [SMILES: CC(=O)N1CCn2c1nc(-c1cccc(C)n1)c2-c1cccc2nnsc12]

Prepared from Intermediate 19 (50 mg, 0.206 mmol) and 7-bromobenzo[d][1,2,3] thiadiazole (66.6 mg, 0.310 mmol) following procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (50 mg, 0.133 mmol, 64% yield) as yellow solid.

LC-MS (ESI): m/z (M+1) 377.0, rt=0.57 min (Method E).

### Intermediate 46:1-(5-(Benzo[c][1,2,5] oxadiazol-4-yl)-6-(6-methylpyridin-2-yl)-2,3-dihydro-1H-imidazo[1,2-a]imidazol-1-yl) ethan-1-one

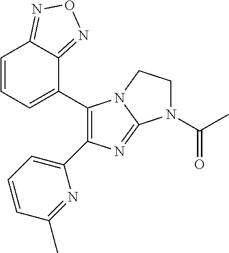 [SMILES: CC(=O)N1CCn2c1nc(-c1cccc(C)n1)c2-c1cccc2nonc12]

0.413 mmol) and 4-Prepared from Intermediate 19 (100 mg, bromobenzo[c][1,2,5] oxadiazole (123 mg, 0.619 mmol) following procedure A. Purification by RP flash chromatography (gradient of elution from 0% to 30% of B in A; A: water/MeCN 95:5+0.1% HCOOH, B: MeCN/water 95:5+0.1% HCOOH) yielded the title compound (79 mg, 0.219 mmol, 53% yield) as a yellow solid.

LC-MS (ESI): m/z (M+1) 361.3, rt=0.52 min (Method E).

### Intermediate 47:1-(5-(benzo[d]thiazol-7-yl)-6-(6-methylpyridin-2-yl)-2,3-dihydro-1H-imidazo[1,2-a]imidazol-1-yl) ethan-1-one

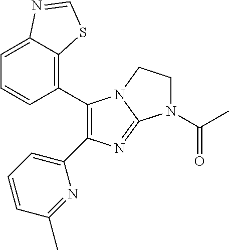 [SMILES: CC(=O)N1CCn2c1nc(-c1cccc(C)n1)c2-c1cccc2ncsc12]

Prepared from Intermediate 19 (100 mg, 0.413 mmol) and 7-bromobenzo[d]thiazole (133 mg, 0.619 mmol) following procedure A. Purification by flash chromatography (gradient of elution from 0 to 50% of EtOAc in heptane) yielded the title compound (110 mg, 0.293 mmol, 71.0% yield) as a yellow solid.

LC-MS (ESI): m/z (M+1) 376.3, rt=0.43 min (Method E).

### Intermediate 48:1-(5-(Benzo[c][1,2,5] thiadiazol-5-yl)-6-(6-methylpyridin-2-yl)-2,3-dihydro-1H-imidazo[1,2-a]imidazol-1-yl) ethan-1-one

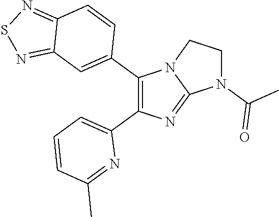 [SMILES: CC(=O)N1CCn2c1nc(-c1cccc(C)n1)c2-c1ccc2nsnc2c1]

Prepared from Intermediate 19 (70 mg, 0.29 mmol) and 5-bromo-2,1,3-benzothiadiazole (93 mg, 0.43 mmol) following procedure A. Purification by RP flash chromatography (gradient of elution from 0 to 80% of B in A; A: water/MeCN 95:5+0.1% HCOOH, B: MeCN/water 95:5+0.1% HCOOH) yielded the title compound (54 mg, 0.14 mmol, 50% yield). LC-MS (ESI): m/z (M+1) 376.8, rt=0.52 min (Method E).

### Intermediate 49:1-(5-(Benzo[c][1,2,5] oxadiazol-5-yl)-6-(6-methylpyridin-2-yl)-2,3-dihydro-1H-imidazo[1,2-a]imidazol-1-yl) ethan-1-one

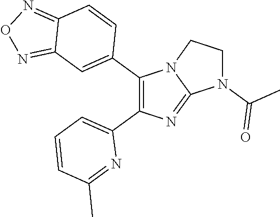 [SMILES: CC(=O)N1CCn2c1nc(-c1cccc(C)n1)c2-c1ccc2nonc2c1]

Prepared from Intermediate 19 (70 mg, 0.29 mmol) and 5-bromobenzo[c][1,2,5] oxadiazole (86 mg, 0.43 mmol) following procedure A. Purification by RP flash chromatography (gradient of elution from 0 to 70% of B in A; A: water/MeCN 95:5+0.1% HCOOH, B: MeCN/water 95:5+0.1% HCOOH) yielded the title compound (55 mg, 0.29 mmol, 53% yield). LC-MS (ESI): m/z (M+1) 360.9, rt=0.60 min (Method E).

### Intermediate 50:1-(5-(benzo[d][1,2,3] thiadiazol-4-yl)-6-(6-methylpyridin-2-yl)-2,3-dihydro-1H-imidazo[1,2-a]imidazol-1-yl) ethan-1-one

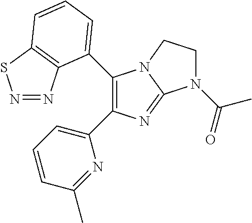 [SMILES: CC(=O)N1CCn2c1nc(-c1cccc(C)n1)c2-c1cccc2snnc12]

Prepared from Intermediate 19 (80 mg, 0.33 mmol) and 4-bromobenzo[d][1,2,3] thiadiazole (107 mg, 0.49 mmol) following procedure A. Purification by RP flash chromatography (gradient of elution from 0 to 80% of B in A; A: water/MeCN 95:5+0.1% HCOOH, B: MeCN/water 95:5+0.1% HCOOH) yielded the title compound (124 mg, 0.085 mmol, 26% yield) as yellow powder.

LC-MS (ESI): m/z (M+1) 376.9, rt=0.46 min (Method E).

### Intermediate 51:1-(5-(benzo[d][1,2,3] thiadiazol-5-yl)-6-(6-methylpyridin-2-yl)-2,3-dihydro-1H-imidazo[1,2-a]imidazol-1-yl) ethan-1-one

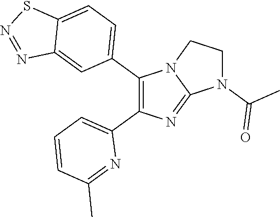 [SMILES: CC(=O)N1CCn2c1nc(-c1cccc(C)n1)c2-c1ccc2snnc2c1]

Prepared from Intermediate 19 (80 mg, 0.33 mmol) and 5-bromo-1,2,3-benzothiadiazole (92 mg, 0.43 mmol) following procedure A. Purification by RP flash chromatography (gradient of elution from 0 to 80% of B in A; A: water/MeCN 95:5+0.1% HCOOH, B: MeCN/water 95:5+0.1% HCOOH) yielded the title compound (30 mg, 0.080 mmol, 24% yield) as beige powder.

LC-MS (ESI): m/z (M+1) 377.3, rt=0.50 min (Method E).

### Intermediate 52: (1-(5-(Benzo[d][1,2,3] thiadiazol-6-yl)-6-(6-methylpyridin-2-yl)-2,3-dihydro-1H-imidazo[1,2-a]imidazol-1-yl) ethan-1-one

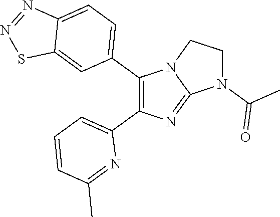 [SMILES: CC(=O)N1CCn2c1nc(-c1cccc(C)n1)c2-c1ccc2nnsc2c1]

Prepared from Intermediate 19 (80 mg, 0.330 mmol) and 6-bromo-1,2,3-benzothiadiazole (92 mg, 0.43 mmol) following procedure A. Purification by RP flash chromatography (gradient of elution from 0 to 80% of B in A; A: water/MeCN 95:5+0.1% HCOOH, B: MeCN/water 95:5+0.1% HCOOH) yielded the title compound (55 mg, 0.147 mmol, 45% yield) as beige powder.

LC-MS (ESI): m/z (M+1) 377.0, rt=0.51 min (Method E).

### Intermediate 53:1-(6-(6-Methylpyridin-2-yl)-5-(4-(methylsulfonyl)phenyl)-2,3-dihydro-1H-imidazo[1,2-a]imidazol-1-yl) ethenone

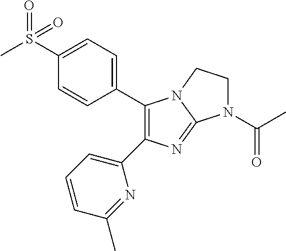 [SMILES: CC(=O)N1CCn2c1nc(-c1cccc(C)n1)c2-c1ccc(S(C)(=O)=O)cc1]

Prepared from Intermediate 19 (80 mg, 0.330 mmol) and 1-bromo-4-(methylsulfonyl)benzene (78 mg, 0.330 mmol) following procedure A. Purification by NH-silica flash chromatography (gradient of elution from 0 to 100% of DCM/MeOH 9:1 in DCM) yielded the title compound (48 mg, 0.121 mmol, 37% yield) as white solid.

LC-MS (ESI): m/z (M+1) 397.1, rt=0.41 min (Method E).

### Intermediate 54:3-(1-Acetyl-6-(6-methylpyridin-2-yl)-2,3-dihydro-1H-imidazo[1,2-a]imidazol-5-yl)benzenesulfonamide

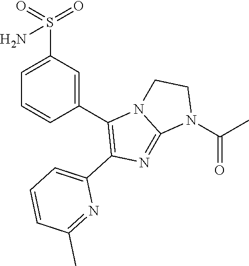 [SMILES: CC(=O)N1CCn2c1nc(-c1cccc(C)n1)c2-c1cccc(S(N)(=O)=O)c1]

Prepared from Intermediate 19 (80 mg, 0.330 mmol) and 3-bromobenzenesulfonamide (78 mg, 0.330 mmol), following procedure A, in the presence of Pd₂(dba)₃ (15.12 mg, 0.017 mmol). Purification by NH-silica flash chromatography (gradient of elution from 0 to 100% of DCM/MeOH 9:1 in DCM) yielded the title compound (63 mg, 0.159 mmol, 48% yield).

LC-MS (ESI): m/z (M+1) 398.3, rt=0.38 min (Method E).

### Intermediate 55: Methyl 5-(1-acetyl-6-(6-methylpyridin-2-yl)-2,3-dihydro-1H-imidazo[1,2-a]imidazol-5-yl)-2-methoxybenzoate

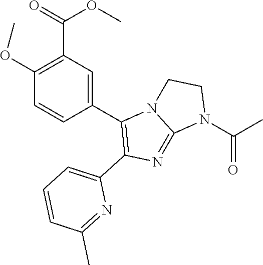 [SMILES: COC(=O)c1cc(-c2c(-c3cccc(C)n3)nc3n2CCN3C(C)=O)ccc1OC]

Prepared from Intermediate 19 (100 mg, 0.413 mmol) and methyl 5-bromo-2-methoxybenzoate (152 mg, 0.619 mmol) following procedure A. Purification by NH-silica flash chromatography (gradient of elution from 0 to 50% of DCM/MeOH 9:1 in DCM) yielded the title compound as crude mixture which was used in the next step without further purification. LC-MS (ESI): m/z (M+1) 407.3, rt=0.48 min (Method E).

### Intermediate 56:1-(5-(3-Amino-4-fluorophenyl)-6-(6-methylpyridin-2-yl)-2,3-dihydro-1H-imidazo[1,2-a]imidazol-1-yl) ethenone

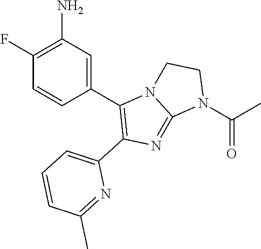 [SMILES: CC(=O)N1CCn2c1nc(-c1cccc(C)n1)c2-c1ccc(F)c(N)c1]

Prepared from Intermediate 19 (80 mg, 0.330 mmol) and 5-bromo-2-fluoroaniline (94 mg, 0.495 mmol), following procedure A, in the presence of Pd₂(dba)₃ (15.12 mg, 0.017 mmol). Purification by NH-silica flash chromatography (gradient of elution from 0 to 100% of DCM/MeOH 9:1 in DCM) yielded the title compound (27 mg, 0.077 mmol, 23% yield) as a brown oil. LC-MS (ESI): m/z (M+1) 352.3, rt=0.42 min (Method E).

### Intermediate 57:1-(6-(6-Methylpyridin-2-yl)-5-(3-(methylthio)phenyl)-2,3-dihydro-1H-imidazo[1,2-a]imidazol-1-yl) ethan-1-one

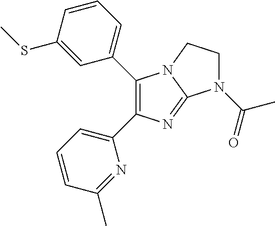 [SMILES: CSc1cccc(-c2c(-c3cccc(C)n3)nc3n2CCN3C(C)=O)c1]

Prepared from Intermediate 19 (400 mg, 1.651 mmol) and (3-bromophenyl)(methyl) sulfane (503 mg, 2.476 mmol) following procedure A. Purification by RP flash chromatography (gradient of elution from 0 to 50% of B in A; A: water/MeCN 95:5+0.1% HCOOH, B: MeCN/water 95:5+0.1% HCOOH) yielded the title compound (287 mg, 0.787 mmol, 48% yield) as a brown solid.

LC-MS (ESI): m/z (M+1) 365.3, rt=0.55 min (Method E).

### Intermediate 58:1-(6-(6-Methylpyridin-2-yl)-5-(4-(methylthio)phenyl)-2,3-dihydro-1H-imidazo[1,2-a]imidazol-1-yl) ethan-1-one

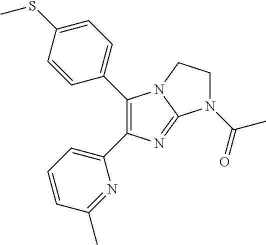 [SMILES: CSc1ccc(-c2c(-c3cccc(C)n3)nc3n2CCN3C(C)=O)cc1]

Prepared from Intermediate 19 (300 mg, 1.238 mmol) and (4-bromophenyl)(methyl) sulfane (377 mg, 1.857 mmol) following procedure A. Purification by flash chromatography (gradient of elution from 0 to 50% EtOAc in heptane) yielded the title compound (100 mg, 0.274 mmol, 22% yield) as a yellow solid

LC-MS (ESI): m/z (M+1) 365.4, rt=0.57 min (Method E).

### Intermediate 59:1-(6-(6-Methylpyridin-2-yl)-5-(3-(methylsulfinyl)phenyl)-2,3-dihydro-1H-imidazo[1,2-a]imidazol-1-yl) ethan-1-one

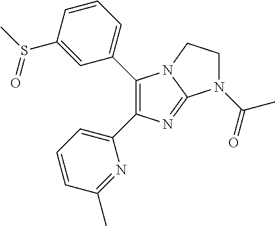 [SMILES: CC(=O)N1CCn2c1nc(-c1cccc(C)n1)c2-c1cccc(S(C)=O)c1]

To a solution of Intermediate 57 (100 mg, 0.274 mmol) in dry DCM (10 mL), m-CPBA (23.67 mg, 0.137 mmol) was added and the reaction was stirred at RT for 12 h. Then, 5 mL of sat. NaHCO₃ solution and Na₂SO₃ were added. The two phases were separated and the aqueous layer was extracted with DCM. Combined extracts were filtered through a phase separator tube and volatiles removed under reduced pressure. Purification by RP flash chromatography (gradient of elution from 0 to 30% of B in A; A: water/MeCN 95:5+0.1% HCOOH, B: MeCN/water 95:5+0.1% HCOOH) yielded the title compound (30 mg, 0.079 mmol, 29% yield) as yellow solid.

LC-MS (ESI): m/z (M+1) 381.4, rt=0.36 min (Method E).

### Intermediate 60:1-(6-(6-Methylpyridin-2-yl)-5-(4-(methylsulfinyl)phenyl)-2,3-dihydro-1H-imidazo[1,2-a]imidazol-1-yl) ethan-1-one

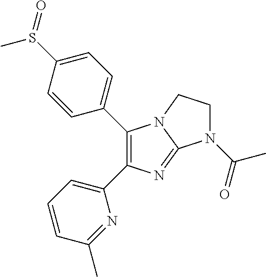 [SMILES: CC(=O)N1CCn2c1nc(-c1cccc(C)n1)c2-c1ccc(S(C)=O)cc1]

Prepared from Intermediate 58 (100 mg, 0.274 mmol) following similar procedure as for Intermediate 59. Purification by RP flash chromatography (gradient of elution from 0 to 30% of B in A; A: water/MeCN 95:5+0.1% HCOOH, B: MeCN/water 95:5+0.1% HCOOH) yielded the title compound (50 mg, 0.131 mmol, 48% yield) as yellow solid.

LC-MS (ESI): m/z (M+1) 381.4, rt=0.40 min (Method E).

### Intermediate 61:1-(6-(6-Methylpyridin-2-yl)-5-(3-(methylsulfonyl)phenyl)-2,3-dihydro-1H-imidazo[1,2-a]imidazol-1-yl) ethan-1-one

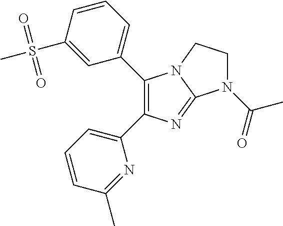 [SMILES: CC(=O)N1CCn2c1nc(-c1cccc(C)n1)c2-c1cccc(S(C)(=O)=O)c1]

To a solution of Intermediate 57 (100 mg, 0.274 mmol) in dry MeCN (10 mL), potassium peroxymonosulfate (0.096 mL, 0.137 mmol) was added and the reaction was heated to 80° C. and stirred for 12 h. The reaction mixture was filtered and the solvent was removed under reduced pressure. Purification by RP flash chromatography (gradient of elution from 0 to 30% of B in A; A: water/MeCN 95:5+0.1% HCOOH, B: MeCN/water 95:5+0.1% HCOOH) yielded the title compound (50 mg, 0.126 mmol, 46% yield) as pale yellow solid. LC-MS (ESI): m/z (M+1) 397.3, rt=0.41 min (Method E).

### Intermediate 62: Methyl 2-bromo-5-(methylthio)benzoate

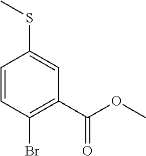

To a solution of methyl 2-bromo-5-fluorobenzoate (1 g, 4.29 mmol) in dry DMF (10 mL), sodium methanethiolate (271 mg, 3.86 mmol) was added and the reaction was stirred at RT for 6 h. Solvent was removed under reduced pressure. Residue was dissolved in DCM (50 mL) and washed with brine, the two phases were separated and the organic layer was filtered through a phase separator tube. Volatiles were removed under reduced pressure. Purification by flash chromatography (gradient of elution from 0 to 5% EtOAc in heptane) yielded the title compound (546 mg, 2.091 mmol, 49% yield) as colourless oil. LC-MS (ESI): m/z (M+1) 261.1-263.0, rt=1.14 min (Method E).

### Intermediate 63: Methyl 2-(1-acetyl-6-(6-methylpyridin-2-yl)-2,3-dihydro-1H-imidazo[1,2-a]imidazol-5-yl)-5-(methylthio)benzoate

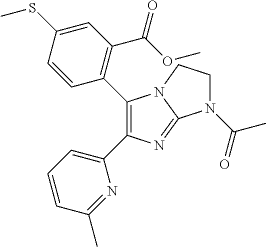 [SMILES: COC(=O)c1cc(SC)ccc1-c1c(-c2cccc(C)n2)nc2n1CCN2C(C)=O]

Prepared from Intermediate 19 (200 mg, 0.825 mmol) and Intermediate 62 (323 mg, 1.238 mmol) following procedure A, in the presence of Pd₂(dba)₃ (76 mg, 0.083 mmol). Purification by RP flash chromatography (gradient of elution from 0 to 30% of B in A; A: water/MeCN 95:5+0.1% HCOOH, B: MeCN/water 95:5+0.1% HCOOH) yielded the title compound (85 mg, 0.201 mmol, 24% yield) as yellow solid.

LC-MS (ESI): m/z (M+1) 423.3, rt=0.58 min (Method E)

### Intermediate 64:1-(6-(6-Methylpyridin-2-yl)-5-(1H-pyrrolo[2,3-b]pyridin-4-yl)-2,3-dihydro-1H-imidazo[1,2-a]imidazol-1-yl) ethan-1-one

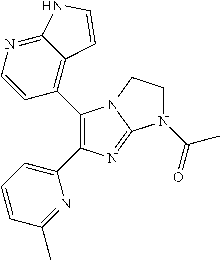 [SMILES: CC(=O)N1CCn2c1nc(-c1cccc(C)n1)c2-c1ccnc2[nH]ccc12]

Prepared from Intermediate 19 (150 mg, 0.619 mmol) and 4-bromo-1H-pyrrolo[2,3-b]pyridine (183 mg, 0.929 mmol) following procedure A. Purification by RP flash chromatography (gradient of elution from 0 to 30% of B in A; A: water/MeCN 95:5+0.1% HCOOH, B: MeCN/water 95:5+0.1% HCOOH) yielded the titled compound (50 mg, 0.140 mmol, 22.53% yield) as yellow solid.

LC-MS (ESI): m/z (M+1) 359.4, rt=0.37 min (Method E).

### Intermediate 65:1-(5-(2-Amino-4-fluorophenyl)-6-(6-methylpyridin-2-yl)-2,3-dihydro-1H-imidazo[1,2-a]imidazol-1-yl) ethan-1-one

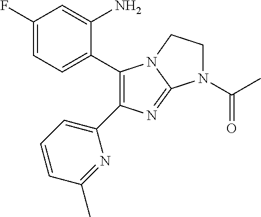 [SMILES: CC(=O)N1CCn2c1nc(-c1cccc(C)n1)c2-c1ccc(F)cc1N]

Prepared from Intermediate 19 (200 mg, 0.825 mmol) and 2-bromo-5-fluoroaniline (235 mg, 1.238 mmol) following procedure A, in the presence of Pd₂(dba)₃ (37.8 mg, 0.041 mmol). Purification by RP flash chromatography (gradient elution from 0 to 45% A in B; A: water/acetonitrile 95:5+0.1% HCOOH, B: water/acetonitrile 5:95+0.1% HCOOH) yielded the title compound (229 mg, 0.652 mmol, 79% yield) as yellow powder. LC-MS (ESI): m/z (M+1) 352.3, rt=0.47 min (Method E).

### Intermediate 66: Ethyl 2-(1-acetyl-6-(6-methylpyridin-2-yl)-5-(thieno[3,2-c]pyridin-2-yl)-2,3-dihydro-1H-imidazo[1,2-a]imidazol-2-yl)acetate

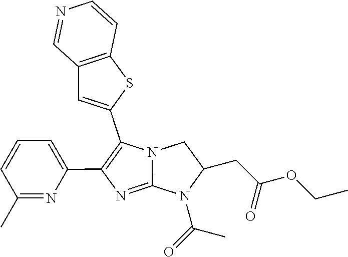 [SMILES: CCOC(=O)CC1Cn2c(nc(-c3cccc(C)n3)c2-c2cc3cnccc3s2)N1C(C)=O]

Prepared from Intermediate 30 (140 mg, 0.385 mmol) and Intermediate 1 (245 mg, 0.576 mmol) following procedure C. Purification by flash chromatography (elution with hexane/EtOAc/Et₃N 1:4:0.01) yielded the title compound (52 mg, 0.113 mmol, 29% yield) as orange solid. UPLC-MS: m/z (M+1) 462.8, rt=1.28 min

### Intermediate 67:1-(5-Bromo-6-(1-methyl-1H-pyrazol-3-yl)-2,3-dihydro-1H-imidazo[1,2-a]imidazol-1-yl) ethan-1-one

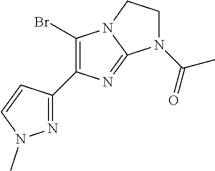 [SMILES: CC(=O)N1CCn2c1nc(-c1ccn(C)n1)c2Br]

Prepared from Intermediate 25 (78 mg, 0.337 mmol) following similar procedure as for Intermediate 20. Purification by RP flash chromatography (gradient of elution from 0% to 100% of B in A; A: water/MeCN 95:5+0.1% HCOOH, B: MeCN/water 95:5+0.1% HCOOH) yielded the title compound (13.5 mg, 0.044 mmol, 13% yield) as off-white solid. LC-MS (ESI): m/z (M+1) 309.8-311.7, rt=0.59 min (Method E).

### Intermediate 68:1-Acetyl-N,N-dimethyl-6-(6-methylpyridin-2-yl)-5-(thieno[3,2-c]pyridin-2-yl)-2,3-dihydro-1H-imidazo[1,2-a]imidazole-2-carboxamide

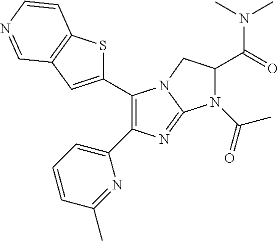 [SMILES: CC(=O)N1c2nc(-c3cccc(C)n3)c(-c3cc4cnccc4s3)n2CC1C(=O)N(C)C]

To a solution of Intermediate 27 (100 mg, 0.318 mmol) in MeOH (5 mL), LiOH (15 mg, 0.637 mmol) dissolved in water (0.5 mL) was added. The reaction was stirred at RT for 30 minutes, then quenched with 1.25M HCl in MeOH. Volatiles were removed under reduced pressure and the crude acid was suspended in SOCl₂ (0.5 mL, 7.003 mmol). The mixture was heated to 70° C. and stirred for 30 min. Volatiles were removed under reduced pressure, the residue taken up with dimethylamine (2M in THF, 2 mL, 2.0 mmol) and stirred at RT for 30 minutes. Volatiles were removed under reduced pressure and the remaining solid was triturated with water (5 mL), filtered and dried to yield the crude dimethyl amide (80 mg, 0.256 mmol). Bromuration according to the procedure as for Intermediate 20, followed by cross-coupling with Intermediate 1 (65 mg, 0.153 mmol) according to procedure C led to the crude Intermediate 68. Purification by pTLC (SiO₂; EtOAc/Hexanes/Et₃N 10:2:0.5) yielded the title compound (15 mg, 0.034 mmol, 33% yield over 4 steps) as yellow solid. UPLC-MS: m/z (M+1) 447.6, rt=1.17 min.

### Intermediate 69:1-(5-(3-amino-4-methoxyphenyl)-6-(6-methylpyridin-2-yl)-2,3-dihydro-1H-imidazo[1,2-a]imidazol-1-yl) ethan-1-one

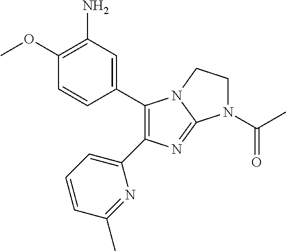 [SMILES: COc1ccc(-c2c(-c3cccc(C)n3)nc3n2CCN3C(C)=O)cc1N]

Prepared from Intermediate 19 (80 mg, 0.330 mmol) and 5-bromo-2-methoxyaniline (100 mg, 0.495 mmol), following procedure A, in the presence of Pd₂(dba)₃ (15.12 mg, 0.017 mmol). Purification by NH-silica flash chromatography (gradient of elution from 0 to 100% of DCM/EtOH 9:1 in DCM) yielded the title compound (40 mg, 0.110 mmol, 33.3% yield).

LC-MS (ESI): m/z (M+1) 364.2, rt=0.40 min (Method E)

### Intermediate 70: Tert-butyl (6-bromobenzo[d]thiazol-2-yl)carbamate

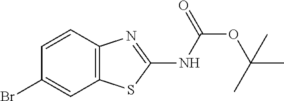 [SMILES: CC(C)(C)OC(=O)Nc1nc2ccc(Br)cc2s1]

A solution of 6-bromobenzo[d]thiazol-2-amine (300 mg, 1.310 mmol), di-tert-butyl dicarbonate ReagentPlus® (429 mg, 1.964 mmol) and DMAP (cat) in DCM (6 mL) was stirred at RT for 12 h. The mixture was washed with a sat solution of NH₄Cl, organic phase filtered through a phase separator tube and concentrated under reduced pressure to give the title compound (388 mg, 1.179 mmol, 90% yield).

LC-MS (ESI): m/z (M-56) 272.9, rt=1.31 min (Method E).

### Intermediate 71: Tert-butyl (6-(4,4,5,5-tetramethyl-1,3,2-dioxaborolan-2-yl)benzo[d] thiazol-2-yl)carbamate

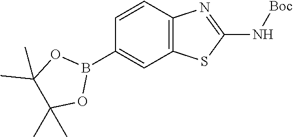 [SMILES: CC(C)(C)OC(=O)Nc1nc2ccc(B3OC(C)(C)C(C)(C)O3)cc2s1]

Prepared followed the procedure for the synthesis of Intermediate 2, starting from Intermediate 70 (238 mg, 0.723 mmol). The mixture was partitioned between DCM and water, the organic phase was dried over Na₂SO₄ and filtered through a phase separator tube. Volatiles were removed under reduced pressure to yield the crude title compound, which was used as such in the next step. LC-MS (ESI): m/z (M+1) 321.2, rt=1.40 min (Method E).

### Intermediate 72: Methyl 5-(1-acetyl-6-(6-methylpyridin-2-yl)-2,3-dihydro-1H-imidazo[1,2-a]imidazol-5-yl)-2-(methylthio)benzoate

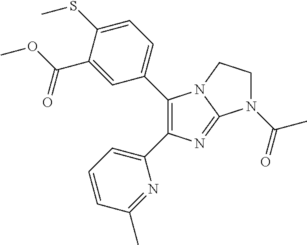 [SMILES: COC(=O)c1cc(-c2c(-c3cccc(C)n3)nc3n2CCN3C(C)=O)ccc1SC]

Prepared from Intermediate 19 (100 mg, 0.413 mmol) and methyl 5-bromo-2-(methylthio)benzoate (269 mg, 1.032 mmol) following procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (45 mg, 0.107 mmol, 26% yield). LC-MS (ESI): m/z (M+1) 423.3, rt=0.58 min (Method E).

### Intermediate 73 66472-1:1-(5-(4-Methoxy-2-nitrophenyl)-6-(6-methylpyridin-2-yl)-2,3-dihydro-1H-imidazo[1,2-a]imidazol-1-yl) ethan-1-one

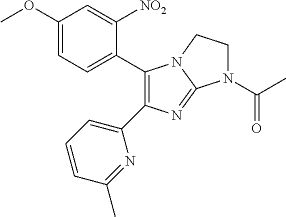 [SMILES: COc1ccc(-c2c(-c3cccc(C)n3)nc3n2CCN3C(C)=O)c([N+](=O)[O-])c1]

Prepared from Intermediate 19 (200 mg, 0.825 mmol) and 1-bromo-4-methoxy-2-nitrobenzene (287 mg, 1.238 mmol) following procedure A, in the presence of Pd₂(dba)₃ (37.8 mg, 0.041 mmol). Purification by RP flash chromatography (gradient of elution from 0% to 40% of B in A; A: water/MeCN 95:5+0.1% HCOOH, B: MeCN/water 95:5+0.1% HCOOH) followed by concentration of fractions and NH-silica flash chromatography (gradient elution from 0 to 40% EtOAc in DMC) yielded the title compound (142 mg, 0.361 mmol, 44% yield).

LC-MS (ESI): m/z (M+1) 394.4, rt=0.55 min (Method E).

### Intermediate 74:1-(5-(2-Amino-4-methoxyphenyl)-6-(6-methylpyridin-2-yl)-2,3-dihydro-1H-imidazo[1,2-a]imidazol-1-yl) ethan-1-one hydrochloride

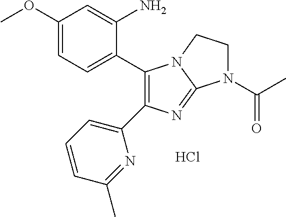 [SMILES: COc1ccc(-c2c(-c3cccc(C)n3)nc3n2CCN3C(C)=O)c(N)c1 ; Cl]

1N HCl (1 mL, 1.000 mmol) was added to a mixture of Intermediate 73 (72 mg, 0.183 mmol) and Fe powder (69.4 mg, 1.243 mmol) in EtOH (3 mL). Stirring went on for 16 h at RT. The reaction mixture was filtered and volatiles were removed under reduced pressure to give the title compound (153 mg, 0.383 mmol, 209% yield). Crude material was used in next step without further purification.

LC-MS (ESI): m/z (M+1) 364.4, rt=0.45 min (Method E).

### Intermediate 75:6-Bromo-2-methoxybenzo[d] thiazole

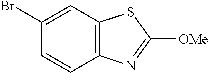 [SMILES: COc1nc2ccc(Br)cc2s1]

To a suspension of NaH (0.322 g, 8.05 mmol) in MeOH (30 mL), 6-bromo-2-chloro-1,3-benzothiazole (2 g, 8.05 mmol) wad added and the solution was stirred for 1 h at 65° C. Volatiles were removed under reduced pressure. Purification by silica gel flash chromatography (gradient elution from 0 to 20% EtOAc in heptane) yielded the title compound (1.9 g, 7.78 mmol, 97% yield). LC-MS (ESI): m/z (M+1) 246.1, rt=1.21 min (Method E).

### Intermediate 76:1-(4-Bromopyridin-2-yl)-3-(2-(4-methylpiperazin-1-yl)ethyl) urea

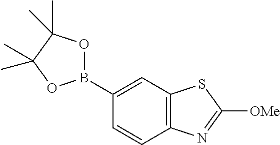 [SMILES: COc1nc2ccc(B3OC(C)(C)C(C)(C)O3)cc2s1]

Prepared followed the procedure for the synthesis of Intermediate 2, starting from Intermediate 75 (3.92 g, 16.06 mmol). Volatiles were removed under reduced pressure and the crude was triturated with EtOAc (100 mL). The solid was filtered and mother liquor was concentrated and the residue was purified by silica gel flash chromatography (gradient elution from 0 to 20% EtOAc in heptane) yielding the title compound (2.08 g, 7.14 mmol, 44.5% yield).

LC-MS (ESI): m/z (M+1) 292.4, rt=1.33 min (Method E).

### Intermediate 77:1-(5-(2-Methoxybenzo[d] thiazol-6-yl)-6-(6-methylpyridin-2-yl)-2,3-dihydro-1H-imidazo[1,2-a]imidazol-1-yl) ethan-1-one

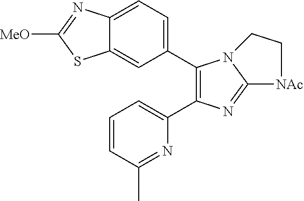

Prepared from Intermediate 19 (300 mg, 0.934 mmol) and Intermediate 76 (408 mg, 1.401 mmol) following procedure B. Purification by silica gel flash chromatography (gradient elution from 0 to 30% EtOAc in heptane) yielded the title compound (225 mg, 0.555 mmol, 59% yield).

### Intermediate 78: (4-Bromo-2-nitrophenyl)(methyl) sulfane

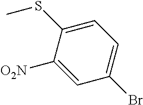

To a solution of 4-bromo-1-fluoro-2-nitrobenzene (0.280 mL, 2.273 mmol) in 2-Propanol (5 mL), sodium methanethiolate (0.191 g, 2.73 mmol), dissolved in Water (1.000 mL), was added and the reaction was stirred at RT for 12 h. 1.2 eq of sodium methanthiolate dissolved in water was then added to push reaction to completion. The reaction was diluted with water and extracted with DCM. The two phases were separated and the aqueous layer extracted with DCM. Combined organic phase was filtered through a Phase Separator tube and the filtrate evaporated under reduced pressure to yield the title compound (0.550 g, 2.217 mmol, 98% yield) as yellow powder.

LC-MS (ESI): m/z (M+1) no ionization, rt=1.16 min (Method E).

### Intermediate 79:5-Bromo-2-(methylthio) aniline

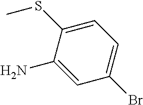

Prepared followed the procedure for the synthesis of Intermediate 74, starting from Intermediate 78 (0.550 g, 2.217 mmol). Purification by flash chromatography (gradient of elution from 0% to 60% of EtOAc in heptane) yielded the title compound (0.295 g, 1.353 mmol, 61% yield). LC-MS (ESI): m/z (M+1) 218.2-220.2, rt=1.06 min (Method E).

### Intermediate 80:1-(5-(3-Amino-4-(methylthio)phenyl)-6-(6-methylpyridin-2-yl)-2,3-dihydro-1H-imidazo[1,2-a]imidazol-1-yl) ethan-1-one

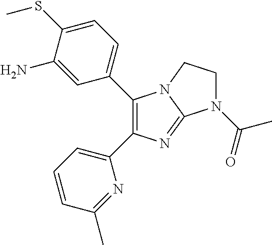 [SMILES: CSc1ccc(-c2c(-c3cccc(C)n3)nc3n2CCN3C(C)=O)cc1N]

Prepared from Intermediate 19 (50 mg, 0.206 mmol) and 5-bromo-2-(methylthio) aniline (0.068 g, 0.310 mmol) following procedure A, in the presence of Pd₂(dba)₃ (10.0 mg, 0.01 mmol). Purification by RP flash chromatography (gradient of elution from 0% to 80% of B in A; A: water/MeCN 95:5+0.1% HCOOH, B: MeCN/water 95:5+0.1% HCOOH) yielded the title compound (10.2 mg, 0.027 mmol, 13% yield).

LC-MS (ESI): m/z (M+1) 380.4, rt=0.51 min (Method E).

### Intermediate 81:1-[5-(4-Methoxyphenyl)-6-(6-methylpyridin-2-yl)-1H,2H,3H-imidazo[1,2-a][1,3]diazol-1-yl]ethan-1-one

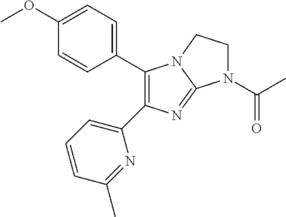 [SMILES: COc1ccc(-c2c(-c3cccc(C)n3)nc3n2CCN3C(C)=O)cc1]

Prepared following procedure B, starting from Intermediate 20 (250 mg, 0.781 mmol) and (4-methoxyphenyl) boronic acid (140 mg, 0.898 mmol). Purification by trituration with MeOH yielded the title compound (220 mg, 0.631 mmol, 81% yield) as a beige solid. UPLC-MS: m/z (M+1) 349, rt=1.39 min. (Method E).

### Intermediate 82:1-[5-(2-Aminopyridin-4-yl)-6-(6-methylpyridin-2-yl)-1H,2H,3H-imidazo[1,2-a][1,3]diazol-1-yl]ethan-1-one

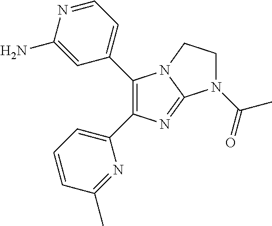 [SMILES: CC(=O)N1CCn2c1nc(-c1cccc(C)n1)c2-c1ccnc(N)c1]

Prepared according procedure B, starting from Intermediate 19 (100.0 mg, 0.310 mmol) and using 4-(4,4,5,5-tetramethyl-1,3,2-dioxaborolan-2-yl) pyridin-2-amine (74.62 mg, 0.340 mmol). The reaction was heated under microwave irradiation to 130° C. for 1 h. Purification by flash chromatography eluting with DCM:MeOH:water (100:3:0.3) yielded the title compound (60 mg, 0.179 mmol, 58.21% yield).

LC-MS (ESI): m/z (M+1): 335.2 (Method F).

### Intermediate 83:2-Bromo-1-(6-methoxypyridin-2-yl) ethan-1-one

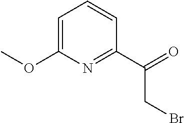

Prepared following the procedure for the synthesis of Intermediate 9, starting from 1-(6-methoxypyridin-2-yl) ethanone (2.0 g, 13.23 mmol) to obtain the title compound (2.19 g, 9.519 mmol, 72% yield). LC-MS (ESI): m/z (M+1): 232.0 (Method E).

### Intermediate 84: N-[4-(6-methoxypyridin-2-yl)-1H-imidazol-2-yl] acetamide

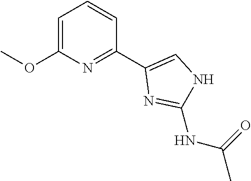 [SMILES: COc1cccc(-c2c[nH]c(NC(C)=O)n2)n1]

Prepared following the procedure for the synthesis of Intermediate 14, starting from Intermediate 83 (2.19 g, 9.52 mmol). Purification by NH-flash chromatography (gradient of elution from 0 to 100% EtOAc in cyclohexane) yielded the title compound (474 mg, 2.041 mmol, 21% yield). LC-MS (ESI): m/z (M+1): 233.1 (Method E).

### Intermediate 85:1-[6-(6-Methoxypyridin-2-yl)-1H,2H,3H-imidazo[1,2-a][1,3]diazol-1-yl]ethan-1-one

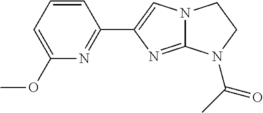 [SMILES: COc1cccc(-c2cn3c(n2)N(C(C)=O)CC3)n1]

Prepared following the procedure for the synthesis of Intermediate 19, starting from Intermediate 84 (244.0 mg, 1.05 mmol) and using 2-methylsulfonyloxyethyl methanesulfonate (275.16 mg, 1.26 mmol) and Cs₂CO₃ (1.52 g, 4.63 mmol). The reaction was stirred at 80° C. for 2 h. The mixture was then diluted with EtOAc and filtered. The filtrate was concentrated under reduced pressure, the residue was triturated with MeOH and the mixture was filtered. The solid was washed with MeOH and dried under vacuum affording title compound (91 mg, 0.352 mmol, 33% yield).

LC-MS (ESI): m/z (M+1): 259.1 (Method E).

### Intermediate 86:1-[5-(1,3-Benzothiazol-6-yl)-6-(6-methoxypyridin-2-yl)-1H,2H,3H-imidazo[1,2-a][1,3]diazol-1-yl]ethan-1-one

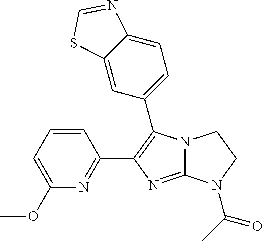 [SMILES: COc1cccc(-c2nc3n(c2-c2ccc4ncsc4c2)CCN3C(C)=O)n1]

Prepared according procedure A, starting from Intermediate 85 (91.0 mg, 0.350 mmol) and using 6-bromobenzothiazole (113.05 mg, 0.530 mmol). Purification by NH-flash chromatography (gradient of elution from 0 to 1.5% of MeOH in DCM) yielded the title compound (223 mg, recovery assumed quantitative).

LC-MS (ESI): m/z (M+1): 392.2 (Method E).

### Preparation of the Examples

### Example 1: [6-(6-Methylpyridin-2-yl)-5-{thieno[3,2-c]pyridin-2-yl}-1H,2H,3H-imidazo[1,2-a][1,3]diazol-2-yl]methanol, single enantiomer

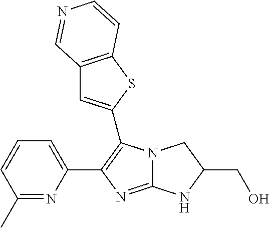 [SMILES: Cc1cccc(-c2nc3n(c2-c2cc4cnccc4s2)CC(CO)N3)n1]

To a solution of Intermediate 36 (160 mg, 0.360 mmol) in EtOH/DCM mixture (5:1), NaBH₄ (108 mg, 2.860 mmol) was added. The reaction was stirred at RT for 3 h, then quenched with brine. The two phases were separated and the aqueous layer was extracted with DCM. Combined organics were dried over Na₂SO₄, filtered and solvent removed under reduced pressure. Sequential trituration with MeCN and MeOH yielded the racemate (52 mg, 0.143 mmol, 40% yield). Semi-preparative chiral separation afforded the title compound as single enantiomer (21 mg, 0.057 mmol, 16% yield) as yellow solid (Method F, 98.8% ee, rt=11.4 min).

LC-MS (ESI): m/z (M+1) 364.2, rt=2.3 min (Method A).

¹H NMR (500 MHz, DMSO-d6) δ ppm 9.04 (d, J=0.8 Hz, 1H), 8.34 (d, J=5.5 Hz, 1H), 7.94-7.97 (m, 1H), 7.93 (s, 1H), 7.64-7.71 (m, 1H), 7.59 (d, J=8.0 Hz, 1H), 7.09 (d, J=7.4 Hz, 1H), 6.65 (d, J=2.7 Hz, 1H), 5.04 (t, J=5.5 Hz, 1H), 4.41-4.49 (m, 1H), 4.26-4.36 (m, 1H), 4.12 (dd, J=9.9, 6.0 Hz, 1H), 3.50-3.62 (m, 2H), 2.53 (s, 3H).

### Example 2:6-(6-(5-Chloro-2-fluorophenyl)-2,3-dihydro-1H-imidazo[1,2-a]imidazol-5-yl)-[1,2,4]triazolo[1,5-a]pyridine

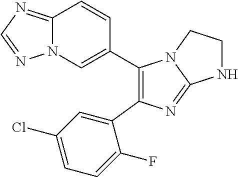 [SMILES: Fc1ccc(Cl)cc1-c1nc2n(c1-c1ccc3ncnn3c1)CCN2]

Prepared according to procedure D, starting from Intermediate 32 (115 mg, 0.290 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (68 mg, 0.192 mmol, 66% yield) as yellowish solid.

LC-MS (ESI): m/z (M+1) 355.0, rt=0.47 min (Method E).

¹H NMR (400 MHZ, DMSO-d6) δ ppm 8.97 (s, 1H) 8.49 (s, 1H) 7.79 (d, J=9.26 Hz, 1H) 7.58 (dd, J=6.17, 2.65 Hz, 1H) 7.44 (dd, J=9.26, 1.32 Hz, 1H) 7.32-7.39 (m, 1H) 7.16 (t, J=9.33 Hz, 1H) 6.77 (s, 1H) 4.22 (br t, J=7.94 Hz, 2H) 3.91 (br t, J=7.72 Hz, 2H).

### Example 3:2-(5-([1,2,4]Triazolo[1,5-a]pyridin-6-yl)-2,3-dihydro-1H-imidazo[1,2-a]imidazol-6-yl)-4-methylthiazole

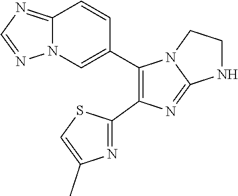 [SMILES: Cc1csc(-c2nc3n(c2-c2ccc4ncnn4c2)CCN3)n1]

Prepared according to procedure D, starting from Intermediate 33 (13.5 mg, 0.037 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (7.5 mg, 0.023 mmol, 63% yield) as yellow solid.

LC-MS (ESI): m/z (M+1) 324.0, rt=0.43 min (Method E).

¹H NMR (400 MHZ, DMSO-d6) δ ppm 9.49 (s, 1H), 8.51 (s, 1H), 7.99 (d, J=9.28 Hz, 1H), 7.84 (d, J=9.26 Hz, 1H), 7.05 (s, 1H), 6.44 (s, 1H), 4.19 (br t, J=7.94 Hz, 2H), 3.86 (br t, J=7.06 Hz, 2H), 2.23 (s, 3H).

### Example 4:6-(6-Methylpyridin-2-yl)-5-{thieno[3,2-c]pyridin-2-yl}-1H,2H,3H-imidazo[1,2-a][1,3]diazole-2-carboxylic acid

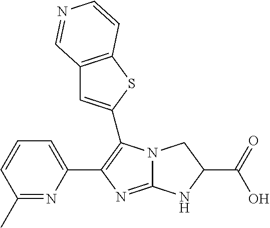 [SMILES: Cc1cccc(-c2nc3n(c2-c2cc4cnccc4s2)CC(C(=O)O)N3)n1]

Prepared according to procedure D, starting from Intermediate 36 (40 mg, 0.067 mmol). Sequential trituration with DCM and Et₂O yielded the title compound (35 mg, 0.078 mmol, 87% yield) as bis hydrochloride salt in form of a yellow solid.

LC-MS (ESI): m/z (M+1) 377.9, rt=2.85 min (Method C).

¹H NMR (400 MHZ, DMSO-d6) δ 9.46 (s, 1H), 8.68 (q, J=6.4 Hz, 2H), 8.24 (s, 1H), 7.88 (t, J=7.8 Hz, 1H), 7.65 (d, J=7.9 Hz, 1H), 7.37 (d, J=7.7 Hz, 1H), 5.15 (dd, J=10.2, 5.9 Hz, 1H), 4.71 (t, J=10.3 Hz, 1H), 4.60 (dd, J=10.1, 5.8 Hz, 1H), 2.61 (s, 3H).

### Example 5:4-(6-(6-Methylpyridin-2-yl)-2,3-dihydro-1H-imidazo[1,2-a]imidazol-5-yl)-2H-pyran-2-one

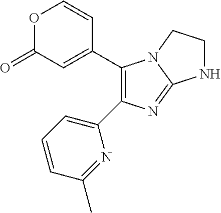 [SMILES: Cc1cccc(-c2nc3n(c2-c2ccoc(=O)c2)CCN3)n1]

Prepared according to procedure D, starting from Intermediate 34 (80 mg, 0.238 mmol). Purification by RP flash chromatography (gradient of elution from 0% to 100% of B in A; A: water/MeCN 95:5+0.1% HCOOH, B: MeCN/water 95:5+0.1% HCOOH), followed by NH-silica flash chromatography (gradient of elution from 0% to 100% of DCM/MeOH 9:1 in DCM) yielded the title compound (2.6 mg, 8.83 μmol, 4% yield) as yellow solid.

LC-MS (ESI): m/z (M+1) 295.2, rt=0.35 min (Method E).

### Example 6:5-(6-(6-Methylpyridin-2-yl)-2,3-dihydro-1H-imidazo[1,2-a]imidazol-5-yl)-2H-pyran-2-one

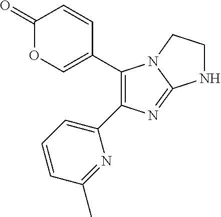 [SMILES: Cc1cccc(-c2nc3n(c2-c2ccc(=O)oc2)CCN3)n1]

Prepared according to procedure D, starting from Intermediate 35 (15 mg, 0.045 mmol). Purification by NH-silica flash chromatography (gradient of elution from 0 to 100% of DCM/MeOH 9:1 in DCM) yielded the title compound (7.8 mg, 0.027 mmol, 59% yield) as yellow solid.

LC-MS (ESI): m/z (M+1) 295.3, rt=0.30 min (Method E).

¹H NMR (600 MHz, METHANOL-d4) δ ppm 8.81 (s, 1H) 8.51 (br d, J=8.72 Hz, 1H) 7.92-7.97 (m, 1H) 7.73 (d, J=15.90 Hz, 1H) 7.65 (br d, J=6.92 Hz, 1H) 6.87 (d, J=15.90 Hz, 1H) 4.73 (br t, J=8.21 Hz, 3H) 4.34 (br t, J=8.14 Hz, 2H) 3.06 (s, 3H).

### Example 7:2-(6-(4-Methylthiazol-2-yl)-2,3-dihydro-1H-imidazo[1,2-a]imidazol-5-yl)thieno[3,2-c]pyridine

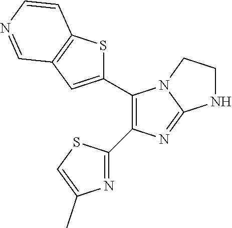 [SMILES: Cc1csc(-c2nc3n(c2-c2cc4cnccc4s2)CCN3)n1]

Prepared according to procedure D, starting from Intermediate 37 (20 mg, 0.052 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (11 mg, 0.032 mmol, 62% yield) as yellow solid.

LC-MS (ESI): m/z (M+1) 340.2, rt=0.41 min (Method E).

¹H NMR (400 MHZ, DMSO-d6) δ ppm 9.10 (s, 1H), 8.47 (s, 1H), 8.39 (br d, J=5.51 Hz, 1H), 8.04 (br d, J=5.29 Hz, 1H), 7.17 (s, 1H), 6.66 (br s, 1H), 4.39 (br t, J=8.05 Hz, 2H), 3.96 (br t, J=7.61 Hz, 2H), 2.44 (s, 3H).

### Example 8:6-(6-(1-Methyl-1H-pyrazol-3-yl)-2,3-dihydro-1H-imidazo[1,2-a]imidazol-5-yl)-[1,2,4]triazolo[1,5-a]pyridine

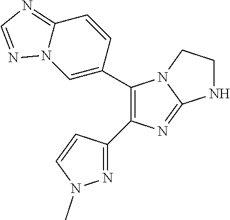 [SMILES: Cn1ccc(-c2nc3n(c2-c2ccc4ncnn4c2)CCN3)n1]

Prepared according to procedure D, starting from Intermediate 38 (123 mg, 0.353 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (108 mg, 0.353 mmol, 100% yield) as white solid.

LC-MS (ESI): m/z (M+1) 307.3, rt=0.31 min (Method E).

¹H NMR (400 MHZ, DMSO-d6) δ ppm 9.31 (s, 1H), 8.52-8.60 (m, 2H), 7.94 (d, J=9.26 Hz, 1H), 7.73 (d, J=8.34 Hz, 2H), 6.26 (br s, 1H), 4.24-4.31 (m, 2H), 4.08-4.13 (m, 2H), 3.81-3.84 (m, 3H).

### Example 9:2-(6-(1-Methyl-1H-pyrazol-3-yl)-2,3-dihydro-1H-imidazo[1,2-a]imidazol-5-yl)thieno[3,2-c]pyridine

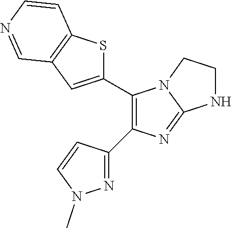 [SMILES: Cn1ccc(-c2nc3n(c2-c2cc4cnccc4s2)CCN3)n1]

Prepared according to procedure D, starting from Intermediate 39 (60 mg, 0.165 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (44 mg, 0.123 mmol, 74% yield) as white solid.

LC-MS (ESI): m/z (M+1) 322.9, rt=0.21 min (Method E).

¹H NMR (400 MHZ, DMSO-d6) δ ppm 9.53 (s, 1H), 8.69 (s, 2H), 8.35 (s, 1H), 7.90 (s, 1H), 6.63 (d, J=1.98 Hz, 1H), 4.47 (br t, J=8.60 Hz, 2H), 4.20 (br t, J=8.60 Hz, 2H), 3.96 (s, 3H).

### Example 10:4-(6-(6-Methylpyridin-2-yl)-2,3-dihydro-1H-imidazo[1,2-a]imidazol-5-yl)benzo[c][1,2,5] thiadiazole

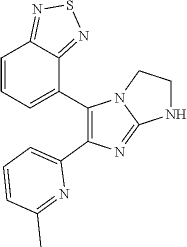 [SMILES: Cc1cccc(-c2nc3n(c2-c2cccc4nsnc24)CCN3)n1]

Prepared according to procedure D, starting from Intermediate 44 (40 mg, 0.106 mmol). Purification by RP flash chromatography (gradient of elution from 0% to 40% of B in A; A: water/MeCN 95:5+0.1% HCOOH, B: MeCN/water 95:5+0.1% HCOOH), followed by SCX (2 g cartridge, elution with 2N NH₃ in methanol) yielded the title compound (25 mg, 0.075 mmol, 70% yield) as a red solid.

LC-MS (ESI): m/z (M+1) 334.8, rt=0.50 min (Method E).

¹H NMR (400 MHZ, DMSO-d6) δ ppm 8.26-8.32 (m, 1H), 7.98-8.04 (m, 1H), 7.82-7.91 (m, 1H), 7.50-7.58 (m, 1H), 7.15-7.20 (m, 1H), 6.92-6.97 (m, 1H), 4.11-4.26 (m, 5H), 2.39 (s, 3H).

### Example 11:4-(6-(6-methylpyridin-2-yl)-2,3-dihydro-1H-imidazo[1,2-a]imidazol-5-yl)benzo[c][1,2,5] oxadiazole

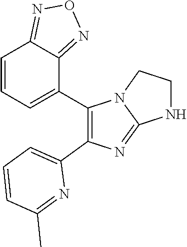 [SMILES: Cc1cccc(-c2nc3n(c2-c2cccc4nonc24)CCN3)n1]

Prepared according to procedure D, starting from Intermediate 46 (79 mg, 0.219 mmol). Purification by RP flash chromatography (gradient of elution from 0% to 30% of B in A; A: water/MeCN 95:5+0.1% HCOOH, B: MeCN/water 95:5+0.1% HCOOH) yielded the title compound (18 mg, 0.057 mmol, 2% yield) as pale yellow solid.

LC-MS (ESI): m/z (M+1) 319.2, rt=0.55 min (Method E).

¹H NMR (400 MHZ, DMSO-d6) δ ppm 7.91 (d, J=8.99 Hz, 1H), 7.70 (d, J=6.65 Hz, 1H), 7.62 (t, J=7.49 Hz, 1H), 7.54 (d, J=4.82 Hz, 2H), 6.83-6.88 (m, 1H), 6.36 (s, 1H), 3.97-4.10 (m, 2H), 3.86 (br t, J=7.67 Hz, 2H), 1.85 (s, 3H).

### Example 12:6-(6-(6-Chloropyridin-2-yl)-2,3-dihydro-1H-imidazo[1,2-a]imidazol-5-yl)-[1,2,4]triazolo[1,5-a]pyridine

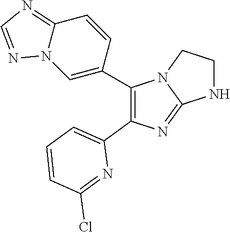 [SMILES: Clc1cccc(-c2nc3n(c2-c2ccc4ncnn4c2)CCN3)n1]

Prepared according to procedure D, starting from Intermediate 40 (175 mg, 0.461 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (39 mg, 0.115 mmol, 25% yield) as white solid.

LC-MS (ESI): m/z (M+1) 338.0, rt=0.41 min (Method E).

¹H NMR (400 MHZ, DMSO-d6) δ ppm 9.39 (s, 1H), 8.64 (s, 1H), 7.98 (br d, J=9.26 Hz, 1H), 7.78-7.84 (m, 1H), 7.77 (d, J=9.26 Hz, 1H), 7.44 (br t, J=8.82 Hz, 2H), 4.22-4.35 (m, 2H), 4.10-4.19 (m, 2H).

### Example 13:2-(6-(6-Chloropyridin-2-yl)-2,3-dihydro-1H-imidazo[1,2-a]imidazol-5-yl)thieno[3,2-c]pyridine

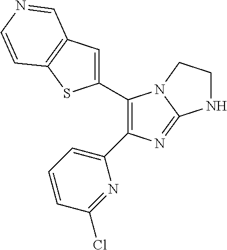 [SMILES: Clc1cccc(-c2nc3n(c2-c2cc4cnccc4s2)CCN3)n1]

Prepared according to procedure D, starting from Intermediate 41 (50 mg, 0.126 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (21 mg, 0.059 mmol, 47% yield) as white solid.

LC-MS (ESI): m/z (M+1) 354.0, rt=0.41 min (Method E)

¹H NMR (400 MHZ, DMSO-d6) δ ppm 9.02 (s, 1H), 8.34 (d, J=5.51 Hz, 1H), 7.95 (d, J=5.51 Hz, 1H), 7.92 (s, 1H), 7.78-7.83 (m, 1H), 7.74 (d, J=7.53 Hz, 1H), 7.28 (d, J=7.72 Hz, 1H), 6.53 (s, 1H), 4.28 (t, J=7.94 Hz, 2H), 3.90 (br t, J=7.28 Hz, 2H).

### Example 14:2-(6-(5-Chloro-2-fluorophenyl)-2,3-dihydro-1H-imidazo[1,2-a]imidazol-5-yl)thieno[3,2-c]pyridine

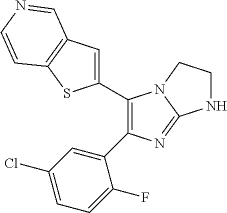 [SMILES: Fc1ccc(Cl)cc1-c1nc2n(c1-c1cc3cnccc3s1)CCN2]

Prepared according to procedure D, starting from Intermediate 42 (188 mg, 0.455 mmol). Purification by RP flash chromatography (gradient of elution from 0% to 100% of B in A; A: water/MeCN 95:5+0.1% HCOOH, B: MeCN/water 95:5+0.1% HCOOH) yielded the title compound (83 mg, 0.224 mmol, 49% yield) as yellow solid.

LC-MS (ESI): m/z (M+1) 371.0, rt=0.41 min (Method E).

¹H NMR (400 MHz, DMSO-d6) δ ppm 9.06 (br s, 1H), 8.37 (br s, 1H), 8.00 (br d, J=4.19 Hz, 1H), 7.53 (s, 1H), 7.46-7.60 (m, 2H), 7.31 (t, J=9.15 Hz, 1H), 6.77 (s, 1H), 4.36 (t, J=8.16 Hz, 2H), 3.99 (br t, J=8.05 Hz, 2H).

### Example 15:1-(6-(6-Methylpyridin-2-yl)-2,3-dihydro-1H-imidazo[1,2-a]imidazol-5-yl)benzo[d] thiazole

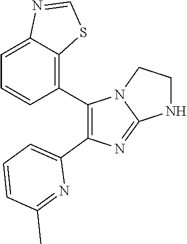 [SMILES: Cc1cccc(-c2nc3n(c2-c2cccc4ncsc24)CCN3)n1]

Prepared according to procedure D, starting from Intermediate 47 (100 mg, 0.413 mmol). Purification by RP flash chromatography (gradient of elution from 0% to 30% of B in A; A: water/MeCN 95:5+0.1% HCOOH, B: MeCN/water 95:5+0.1% HCOOH), followed by NH-silica flash chromatography (gradient of elution from 0 to 5% of EtOH in DCM) yielded the title compound (4 mg, 0.012 mmol, 3% yield) as yellow solid.

LC-MS (ESI): m/z (M+1) 333.9, rt=0.46 min (Method E).

¹H NMR (400 MHZ, DMSO-d6) δ ppm 9.36 (s, 1H), 8.22 (br d, J=6.80 Hz, 1H), 7.65-7.79 (m, 2H), 7.54 (t, J=7.78 Hz, 1H), 7.08 (m, J=7.00 Hz, 2H), 4.07 (br s, 4H), 2.24 (br s, 3H).

### Example 16:7-(6-(6-Methylpyridin-2-yl)-2,3-dihydro-1H-imidazo[1,2-a]imidazol-5-yl)benzo[d][1,2,3] thiadiazole

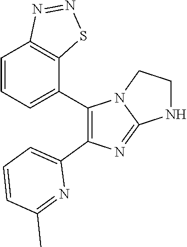 [SMILES: Cc1cccc(-c2nc3n(c2-c2cccc4nnsc24)CCN3)n1]

Prepared according to procedure D, starting from Intermediate 45 (50 mg, 0.133 mmol). Purification by RP flash chromatography (gradient of elution from 0% to 40% of B in A; A: water/MeCN 95:5+0.1% HCOOH, B: MeCN/water 95:5+0.1% HCOOH), followed by SCX (2 g cartridge, elution with 2N NH₃ in methanol) yielded the title compound (10.2 mg, 0.031 mmol, 23% yield) as brown solid.

LC-MS (ESI): m/z (M+1) 334.9, rt=0.51 min (Method E).

¹H NMR (400 MHz, DMSO-d6) δ ppm 8.67 (d, J=8.16 Hz, 1H), 7.91 (d, J=7.06 Hz, 1H), 7.78-7.87 (m, 1H), 7.57-7.66 (m, 2H), 6.87 (d, J=6.84 Hz, 1H), 6.35-6.42 (m, 1H), 3.94-4.07 (m, 2H), 3.82-3.92 (m, 2H). 1.66 (s, 3H)

### Example 17:5-(6-(6-Methylpyridin-2-yl)-2,3-dihydro-1H-imidazo[1,2-a]imidazol-5-yl)benzo[c][1,2,5] thiadiazole

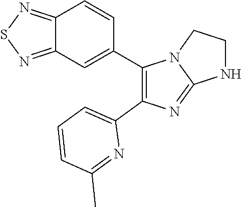 [SMILES: Cc1cccc(-c2nc3n(c2-c2ccc4nsnc4c2)CCN3)n1]

Prepared according to procedure D, starting from Intermediate 48 (54 mg, 0.14 mmol). Purification by RP flash chromatography (gradient of elution from 0% to 20% of B in A; A: water/MeCN 95:5+0.1% HCOOH, B: MeCN/water 95:5+0.1% HCOOH), followed by trituration with DCM and MeCN yielded the title compound (21 mg, 0.063 mmol, 44% yield) as dark red powder.

LC-MS (ESI): m/z (M+1) 335.0, rt=0.55 min (Method E).

¹H NMR (400 MHZ, DMSO-d6) δ ppm 8.22 (s, 1H), 7.89-7.97 (m, 2H), 7.58-7.65 (m, 2H), 7.01 (d, J=6.62 Hz, 1H), 6.37 (s, 1H), 4.15-4.24 (m, 2H), 3.84-3.95 (m, 2H), 2.23 (s, 3H).

### Example 18:5-(6-(6-methylpyridin-2-yl)-2,3-dihydro-1H-imidazo[1,2-a]imidazol-5-yl)benzo[c][1,2,5] oxadiazole

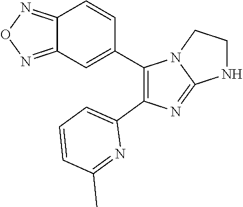 [SMILES: Cc1cccc(-c2nc3n(c2-c2ccc4nonc4c2)CCN3)n1]

Prepared according to procedure D, starting from Intermediate 49 (55 mg, 0.15 mmol). Purification by RP flash chromatography (gradient of elution from 0% to 20% of B in A; A: water/MeCN 95:5+0.1% HCOOH, B: MeCN/water 95:5+0.1% HCOOH) yielded the title compound (37 mg, 0.12 mmol, 76% yield) as red powder.

LC-MS (ESI): m/z (M+1) 318.9, rt=0.53 min (Method E).

¹H NMR (400 MHz, DMSO-d6) δ ppm 8.02 (s, 1H), 7.83 (d, J=9.26 Hz, 1H), 7.68 (dd, J=9.48, 1.10 Hz, 1H), 7.55-7.66 (m, 2H), 7.01 (dd, J=6.73, 1.43 Hz, 1H), 6.44 (br s, 1H), 4.12-4.24 (m, 2H), 3.81-3.92 (m, 2H), 2.22 (s, 3H).

### Example 19: N-methyl-6-(6-methylpyridin-2-yl)-5-{thieno[3,2-b]pyridin-2-yl}-1H,2H,3H-imidazo[1,2-a][1,3]diazole-2-carboxamide

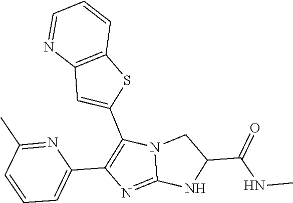

To a solution of Intermediate 36 (40 mg, 0.089 mmol) in THF (2 mL), methylamine (2.0M in THF, 3 mL, 6 mmol) was added. The reaction was stirred at RT for 1 h. Volatiles were removed under reduced pressure and the residue was triturated with MeOH to give the title compound (13 mg, 0.033 mmol, 37% yield) as yellow powder.

LC-MS (ESI): m/z (M+1) 390.9, rt=2.33 min (Method A).

¹H NMR (400 MHZ, DMSO-d6) δ 9.04 (d, J=1.0 Hz, 1H), 8.34 (d, J=5.5 Hz, 1H), 8.19 (d, J=4.8 Hz, 1H), 7.99-7.91 (m, 2H), 7.69 (t, J=7.7 Hz, 1H), 7.60 (d, J=7.8 Hz, 1H), 7.11 (d, J=7.4 Hz, 1H), 7.02 (d, J=4.0 Hz, 1H), 4.76 (ddd, J=10.0, 6.0, 4.0 Hz, 1H), 4.66 (t, J=9.9 Hz, 1H), 4.30 (dd, J=9.9, 6.0 Hz, 1H), 2.68 (d, J=4.7 Hz, 3H), 2.54 (s, 3H).

### Example 20: Ethyl 2-[6-(6-methylpyridin-2-yl)-5-{thieno[3,2-c]pyridin-2-yl}-1H,2H,3H-imidazo[1,2-a][1,3]diazol-2-yl] acetate

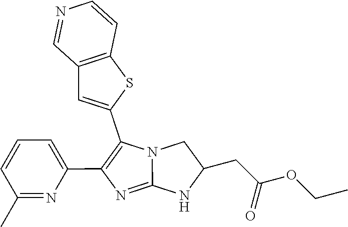 [SMILES: CCOC(=O)CC1Cn2c(nc(-c3cccc(C)n3)c2-c2cc3cnccc3s2)N1]

Prepared according to procedure D, starting from Intermediate 66 (52 mg, 0.113 mmol). Purification by pTLC (NH-silica; DCM/EtOH 99:1) yielded the title compound (15 mg, 0.036 mmol, 32% yield) as yellow powder.

LC-MS (ESI): m/z (M+1) 419.9, rt=2.66 min (Method A).

¹H NMR (400 MHZ, DMSO-d6) δ 9.04 (d, J=1.0 Hz, 1H), 8.36 (d, J=5.5 Hz, 1H), 7.98 (dt, J=5.6, 0.9 Hz, 1H), 7.92 (d, J=0.8 Hz, 1H), 7.66 (t, J=7.7 Hz, 1H), 7.56 (d, J=7.8 Hz, 1H), 7.08 (d, J=7.5 Hz, 1H), 6.60 (s, 1H), 5.14 (dt, J=8.3, 4.6 Hz, 1H), 4.20-4.11 (m, 1H), 3.91-3.82 (m, 2H), 3.74-3.66 (m, 1H), 2.46 (s, 3H), 1.04 (t, J=7.1 Hz, 3H).

### Example 21:2-[6-(6-Methylpyridin-2-yl)-5-{thieno[3,2-c]pyridin-2-yl}-1H,2H,3H-imidazo[1,2-a][1,3]diazol-2-yl]acetic acid

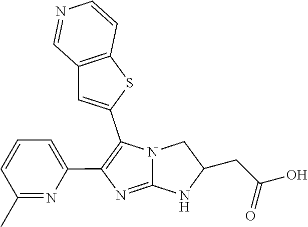 [SMILES: Cc1cccc(-c2nc3n(c2-c2cc4cnccc4s2)CC(CC(=O)O)N3)n1]

Prepared according to procedure D, starting from Example 20 (8 mg, 0.019 mmol). Trituration with triturated with DCM and Et₂O yielded the hydrochloride salt of the title compound (5 mg, 0.012 mmol, 61% yield) as an orange solid.

LC-MS (ESI): m/z (M+1) 391.9, rt=2.34 min (Method A).

¹H NMR (400 MHZ, DMSO-d6) δ 9.50 (s, 1H), 8.68 (s, 2H), 8.23 (s, 1H), 7.78 (t, J=7.8 Hz, 1H), 7.44 (d, J=8.0 Hz, 1H), 7.31 (d, J=7.7 Hz, 1H), 5.32-5.08 (m, 1H), 4.40 (t, J=9.7 Hz, 1H), 3.95 (dd, J=10.2, 4.8 Hz, 1H), 2.76-2.63 (m, 2H), 2.54 (s, 3H).

### Example 22:6-(1-Ethyl-6-(6-methylpyridin-2-yl)-2,3-dihydro-1H-imidazo[1,2-a]imidazol-5-yl)-[1,2,4]triazolo[1,5-a]pyridine

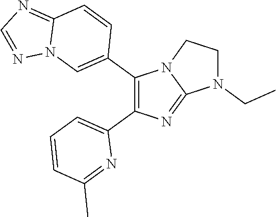 [SMILES: CCN1CCn2c1nc(-c1cccc(C)n1)c2-c1ccc2ncnn2c1]

Prepared from Intermediate 43 (60 mg, 0.263 mmol) and 6-bromo-[1,2,4]triazolo[1,5-a]pyridine (78 mg, 0.394 mmol) following procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (26.3 mg, 0.076 mmol, 29% yield) as yellow solid.

LC-MS (ESI): m/z (M+1) 346.2, rt=0.70 min (Method E).

¹H NMR (400 MHZ, DMSO-d6) δ ppm 9.36 (s, 1H), 8.51 (s, 1H), 7.76-7.90 (m, 2H), 7.56-7.68 (m, 2H), 7.00 (dd, J=5.50, 2.90 Hz, 1H), 4.14 (br t, J=7.50 Hz, 2H), 3.77 (br t, J=7.61 Hz, 2H), 3.27 (q, J=1.00 Hz, 2H), 2.25 (s, 3H), 1.23 (t, J=7.28 Hz, 3H).

### Example 23:6-(1-Methyl-1H-pyrazol-3-yl)-5-(4-(methylthio)phenyl)-2,3-dihydro-1H-imidazo[1,2-a]imidazole

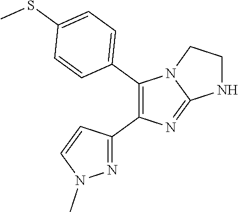 [SMILES: CSc1ccc(-c2c(-c3ccn(C)n3)nc3n2CCN3)cc1]

Prepared from Intermediate 25 (130 mg, 0.562 mmol) and (4-bromophenyl)(methyl) sulfane (114 mg, 0.562 mmol) following procedure A. Purification by RP flash chromatography (gradient of elution from 0% to 100% of B in A; A: water/MeCN 95:5+0.1% HCOOH, B: MeCN/water 95:5+0.1% HCOOH) yielded the title compound (32 mg, 0.103 mmol, 18% yield) as orange solid

LC-MS (ESI): m/z (M+1) 312.0, rt=0.59 min (Method E).

¹H NMR (400 MHZ, DMSO-d6) δ ppm 7.73 (d, J=2.21 Hz, 1H), 7.51 (d, J=8.38 Hz, 2H), 7.37 (d, J=8.16 Hz, 2H), 6.17 (s, 1H), 4.20-4.25 (m, 2H), 4.06-4.14 (m, 2H), 3.87 (s, 3H), 2.52 (s, 3H).

### Example 24:5-(Benzo[d][1,3]dioxol-5-yl)-6-(1-methyl-1H-pyrazol-3-yl)-2,3-dihydro-1H-imidazo[1,2-a]imidazole

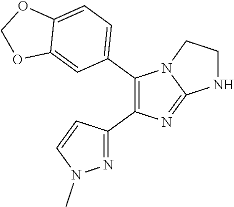 [SMILES: Cn1ccc(-c2nc3n(c2-c2ccc4c(c2)OCO4)CCN3)n1]

Prepared from Intermediate 67 (21 mg, 0.061 mmol) and 2-(benzo[d][1,3]dioxol-5-yl)-4,4,5,5-tetramethyl-1,3,2-dioxaborolane (13.5 mg, 0.081 mmol) following procedure B. Purification by RP flash chromatography (gradient of elution from 0% to 100% of B in A; A: water/MeCN 95:5+0.1% HCOOH, B: MeCN/water 95:5+0.1% HCOOH) yielded the title compound (4 mg, 0.013 mmol, 19% yield) as orange solid.

LC-MS (ESI): m/z (M+1) 310.0, rt=0.49 min (Method E).

¹H NMR (400 MHZ, DMSO-d6) δ ppm 7.54 (d, J=1.98 Hz, 1H), 7.20 (d, J=1.32 Hz, 1H), 6.99-7.08 (m, 1H) 6.90 (d, J=8.16 Hz, 1H), 6.19 (d, J=1.98 Hz, 1H), 3.94-4.11 (m, 2H), 3.78-3.85 (m, 2H) 3.76 (s, 2H), 2.54 (s, 3H).

### Example 25: Ethyl 1-acetyl-6-(6-methylpyridin-2-yl)-5-{[1,2,4]triazolo[1,5-a]pyridin-6-yl}-1H,2H,3H-imidazo[1,2-a][1,3]diazole-2-carboxylate

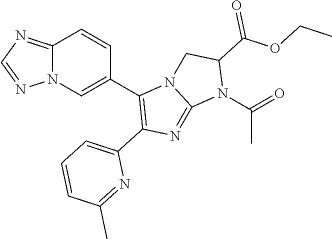 [SMILES: CCOC(=O)C1Cn2c(nc(-c3cccc(C)n3)c2-c2ccc3ncnn3c2)N1C(C)=O]

Prepared from Intermediate 28 (150 mg, 0.383 mmol) and Intermediate 2 (141 mg, 0.574 mmol) following procedure B. Purification by pTLC (SiO₂; EtOAc/Hex/Et₃N 8:2:0.5) yielded the title compound (60 mg, 0.139 mmol, 36% yield) as white powder.

LC-MS (ESI): m/z (M+1) 431.9, rt=1.73 min (Method B).

¹H NMR (400 MHZ, DMSO-d6) δ 9.41 (t, J=1.4 Hz, 1H), 8.56 (s, 1H), 7.85 (d, J=1.2 Hz, 2H), 7.75-7.65 (m, 2H), 7.08 (dd, J=6.8, 1.9 Hz, 1H), 5.40 (dd, J=10.1, 3.9 Hz, 1H), 4.65 (t, J=10.4 Hz, 1H), 4.42 (dd, J=10.6, 4.1 Hz, 1H), 4.18 (q, J=7.1 Hz, 2H), 2.64 (s, 3H), 2.24 (s, 3H), 1.22 (t, J=7.1 Hz, 3H).

### Example 26: Ethyl 6-(6-methylpyridin-2-yl)-5-{[1,2,4]triazolo[1,5-a]pyridin-6-yl}-1H,2H,3H-imidazo[1,2-a][1,3]diazole-2-carboxylate

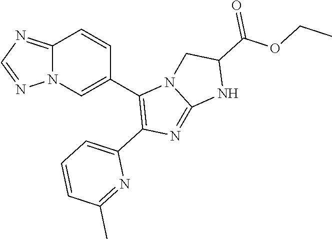 [SMILES: CCOC(=O)C1Cn2c(nc(-c3cccc(C)n3)c2-c2ccc3ncnn3c2)N1]

Prepared according to procedure D, starting from Example 25 (15 mg, 0.035 mmol). Trituration with EtOH yielded the title compound (11 mg, 0.028 mmol, 81% yield) as yellow powder.

LC-MS (ESI): m/z (M+1) 390.0, rt=2.79 min (Method A).

¹H NMR (400 MHZ, DMSO-d6) δ 9.43 (d, J=1.4 Hz, 1H), 8.93 (s, 1H), 8.63 (s, 1H), 7.97 (dd, J=9.3, 1.0 Hz, 1H), 7.84-7.70 (m, 2H), 7.34 (dd, J=19.9, 7.9 Hz, 2H), 5.20 (dd, J=10.5, 5.8 Hz, 1H), 4.61 (t, J=10.5 Hz, 1H), 4.46 (dd, J=10.5, 5.9 Hz, 1H), 4.22 (qd, J=7.1, 2.7 Hz, 2H), 3.57 (s, 3H), 1.25 (t, J=7.1 Hz, 3H).

### Example 27:1-(5-([1,2,4]Triazolo[1,5-a]pyridin-6-yl)-6-(6-methylpyridin-2-yl)-2,3-dihydro-1H-imidazo[1,2-a]imidazol-1-yl) ethan-1-one

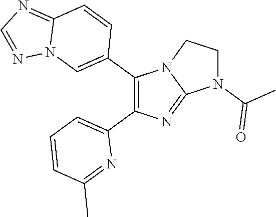 [SMILES: CC(=O)N1CCn2c1nc(-c1cccc(C)n1)c2-c1ccc2ncnn2c1]

Prepared from Intermediate 20 (100 mg, 0.312 mmol) and Intermediate 2 (84 mg, 0.344 mmol) following procedure B. Purification by trituration with anhydrous MeOH yielded the title compound (93 mg, 0.259 mmol, 80% yield) as a white powder.

UPLC-MS: m/z (M+1) 360.3, rt=1.21 min.

¹H NMR (300 MHz, DMSO-d6) δ 9.39 (t, J=1.3 Hz, 1H), 8.56 (s, 1H), 7.87 (d, J=1.3 Hz, 2H), 7.75-7.62 (m, 2H), 7.11-7.00 (m, 1H), 4.42-4.32 (m, 2H), 4.32-4.21 (m, 2H), 2.60 (s, 3H), 2.25 (s, 3H).

### Example 28:4-(6-(6-Methylpyridin-2-yl)-2,3-dihydro-1H-imidazo[1,2-a]imidazol-5-yl)benzo[d][1,2,3] thiadiazole

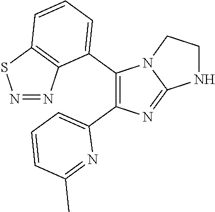 [SMILES: Cc1cccc(-c2nc3n(c2-c2cccc4snnc24)CCN3)n1]

Prepared according to procedure D, starting from Intermediate 50 (32 mg, 0.085 mmol). Purification by SCX (1 g cartridge, elution with 7N NH₃ in MeOH) yielded the title compound (28 mg, 0.084 mmol, 98% yield) as orange powder.

LC-MS (ESI): m/z (M+1) 334.9, rt=0.50 min (Method E).

¹H NMR (400 MHZ, DMSO-d6) δ ppm 8.33 (d, J=7.72 Hz, 1H), 7.76-7.87 (m, 2H), 7.50-7.61 (m, 2H), 6.84 (d, J=7.06 Hz, 1H), 6.23-6.44 (m, 1H), 3.97-4.04 (m, 2H), 3.84-3.94 (m, 2H), 1.78 (s, 3H).

### Example 29: N,N-Dimethyl-6-(6-methylpyridin-2-yl)-5-{thieno[3,2-c]pyridin-2-yl}-1H,2H,3H-imidazo[1,2-a][1,3]diazole-2-carboxamide

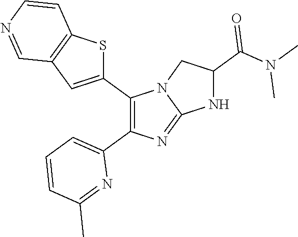 [SMILES: Cc1cccc(-c2nc3n(c2-c2cc4cnccc4s2)CC(C(=O)N(C)C)N3)n1]

Prepared according to procedure D, starting from Intermediate 68 (15 mg, 0.034 mmol). Purification by pTLC (NH-silica; DCM/MeOH 95:5) yielded the title compound (7 mg, 0.017 mmol, 52% yield) as yellow powder.

LC-MS (ESI): m/z (M+1) 404.9, rt=2.32 min (Method A).

¹H NMR (400 MHZ, Chloroform-d) δ 9.02 (d, J=1.1 Hz, 1H), 8.41 (d, J=5.6 Hz, 1H), 7.79-7.73 (m, 1H), 7.73-7.68 (m, 1H), 7.58 (dt, J=15.3, 7.8 Hz, 2H), 7.02 (d, J=7.3 Hz, 1H), 5.11 (d, J=7.6 Hz, 1H), 4.60 (t, J=9.7 Hz, 1H), 4.34 (dd, J=9.8, 6.2 Hz, 1H), 3.07 (d, J=15.0 Hz, 6H), 2.55 (s, 3H).

### Example 30:5-(6-(6-Methylpyridin-2-yl)-2,3-dihydro-1H-imidazo[1,2-a]imidazol-5-yl)benzo[d][1,2,3] thiadiazole

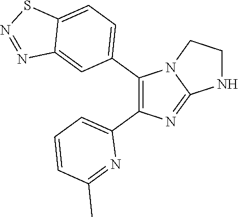 [SMILES: Cc1cccc(-c2nc3n(c2-c2ccc4snnc4c2)CCN3)n1]

Prepared according to procedure D, starting from Intermediate 51 (30 mg, 0.080 mmol). Purification by SCX (1 g cartridge, elution with 7N NH₃ in MeOH) yielded the title compound (26 mg, 0.077 mmol, 97% yield) as yellow powder.

LC-MS (ESI): m/z (M+1) 335.3, rt=0.54 min (Method E).

¹H NMR (400 MHZ, DMSO-d6) δ ppm 9.00 (s, 1H), 8.34 (d, J=8.60 Hz, 1H), 8.06 (dd, J=8.38, 1.32 Hz, 1H), 7.56-7.74 (m, 2H), 6.99 (dd, J=6.50, 1.87 Hz, 1H), 6.33 (br s, 1H), 4.09-4.30 (m, 2H), 3.76-4.01 (m, 2H), 2.20 (s, 3H).

### Example 31:6-(6-(6-Methylpyridin-2-yl)-2,3-dihydro-1H-imidazo[1,2-a]imidazol-5-yl)benzo[d][1,2,3] thiadiazole

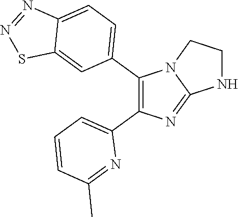 [SMILES: Cc1cccc(-c2nc3n(c2-c2ccc4nnsc4c2)CCN3)n1]

Prepared according to procedure D, starting from Intermediate 52 (55 mg, 0.147 mmol). Purification by SCX (1 g cartridge, elution with 7N NH₃ in MeOH) yielded the title compound (45 mg, 0.135 mmol, 91% yield) as yellow powder.

LC-MS (ESI): m/z (M+1) 334.9, rt=0.52 min (Method E).

¹H NMR (400 MHZ, DMSO-d6) δ ppm 8.63 (s, 1H), 8.59 (d, J=8.82 Hz, 1H), 7.97 (d, J=8.82 Hz, 1H), 7.47-7.71 (m, 2H), 7.01 (d, J=7.06 Hz, 1H), 6.39 (s, 1H), 4.05-4.23 (m, 2H), 3.80-4.00 (m, 2H), 2.21 (s, 3H).

### Example 32:6-(6-methylpyridin-2-yl)-5-(4-(methylsulfonyl)phenyl)-2,3-dihydro-1H-imidazo[1,2-a]imidazole

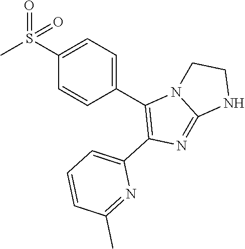 [SMILES: Cc1cccc(-c2nc3n(c2-c2ccc(S(C)(=O)=O)cc2)CCN3)n1]

Prepared according to procedure D, starting from Intermediate 54 (48 mg, 0.121 mmol). Purification by SCX (2 g cartridge, elution with 2N NH₃ in MeOH) yielded the title compound (37.9 mg, 0.107 mmol, 88% yield) as yellow solid.

LC-MS (ESI): m/z (M+1) 354.9, rt=0.41 min (Method E).

¹H NMR (400 MHZ, DMSO-d6) δ ppm 7.80-7.90 (m, 4H), 7.49-7.61 (m, 2H), 6.98 (d, J=7.02 Hz, 1H), 6.31 (t, J=2.19 Hz, 1H), 4.03-4.12 (m, 2H), 3.84 (td, J=7.84, 2.30 Hz, 2H), 3.20 (s, 3H), 2.25 (s, 3H).

### Example 33:6-[1-Ethyl-6-(6-methylpyridin-2-yl)-1H,2H,3H-imidazo[1,2-a][1,3]diazol-5-yl]-1,3-benzothiazole

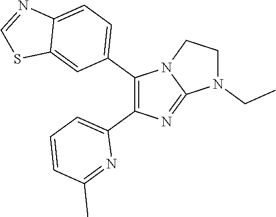 [SMILES: CCN1CCn2c1nc(-c1cccc(C)n1)c2-c1ccc2ncsc2c1]

Prepared from Intermediate 43 (60 mg, 0.263 mmol) and 2-bromo-1,3-benzothiazole (73 mg, 0.342 mmol) following procedure A. Purification by preparative HPLC (water/MeCN gradient+HCOOH) yielded the title compound (16 mg, 0.044 mmol, 17% yield) as a yellow powder.

LC-MS (ESI): m/z (M+1) 361.9, rt=2.01 min (Method B).

¹H NMR (400 MHZ, Methanol-d₄) δ 9.24 (s, 1H), 8.16 (d, J=1.7 Hz, 1H), 8.00 (d, J=8.5 Hz, 1H), 7.62 (t, J=7.8 Hz, 1H), 7.53 (dd, J=8.6, 1.8 Hz, 1H), 7.35 (d, J=7.9 Hz, 1H), 7.10 (d, J=7.7 Hz, 1H), 4.17 (dd, J=8.6, 7.0 Hz, 2H), 3.89 (dd, J=8.5, 7.0 Hz, 2H), 3.41 (q, J=7.2 Hz, 2H), 2.37 (s, 3H), 1.29 (t, J=7.2 Hz, 3H).

### Example 34:6-(6-Methylpyridin-2-yl)-5-(4-(methylsulfinyl)phenyl)-2,3-dihydro-1H-imidazo[1,2-a]imidazole

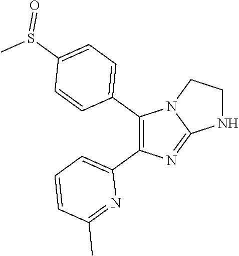 [SMILES: Cc1cccc(-c2nc3n(c2-c2ccc(S(C)=O)cc2)CCN3)n1]

Prepared according to procedure D, starting from Intermediate 60 (50 mg, 0.131 mmol). Purification by RP flash chromatography (gradient of elution from 0% to 30% of B in A; A: water/MeCN 95:5+0.1% HCOOH, B: MeCN/water 95:5+0.1% HCOOH) followed by NH-silica flash chromatography (gradient of elution from 0 to 5% of EtOH in DCM) yielded the title compound (9 mg, 0.027 mmol, 20% yield) as yellow solid.

LC-MS (ESI): m/z (M+1) 339.2, rt=0.36 min (Method E).

¹H NMR (400 MHZ, DMSO-d6) δ ppm 7.84 (d, J=8.33 Hz, 2H), 7.64 (d, J=8.33 Hz, 2H), 7.52-7.62 (m, 2H), 6.99 (d, J=7.23 Hz, 1H), 6.22-6.29 (m, 1H), 4.02-4.15 (m, 2H), 3.80-3.92 (m, 2H), 2.77 (s, 3H), 2.27 (s, 3H).

### Example 35:4-(6-(6-Methylpyridin-2-yl)-2,3-dihydro-1H-imidazo[1,2-a]imidazol-5-yl)-1H-pyrrolo[2,3-b]pyridine

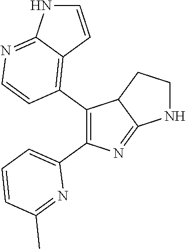 [SMILES: Cc1cccc(C2=C(c3ccnc4[nH]ccc34)C3CCNC3=N2)n1]

Prepared following procedure D, starting from Intermediate 64 (50 mg, 0.140 mmol). Purification by RP flash chromatography (gradient of elution from 0% to 30% of B in A; A: water/MeCN 95:5+0.1% HCOOH, B: MeCN/water 95:5+0.1% HCOOH) followed by NH-silica flash chromatography (gradient of elution from 0 to 5% of EtOH in DCM) yielded the title compound (10 mg, 0.032 mmol) as yellow solid.

LC-MS (ESI): m/z (M+1) 317.3, rt=0.38 min (Method E).

¹H NMR (400 MHZ, DMSO-d6) δ ppm 11.55 (br s, 1H), 8.19 (d, J=4.82 Hz, 1H), 7.54 (t, J=7.67 Hz, 1H), 7.42 (d, J=7.89 Hz, 1H), 7.35 (t, J=2.85 Hz, 1H), 7.21 (d, J=5.04 Hz, 1H), 6.92 (d, J=7.67 Hz, 1H), 6.38 (br s, 1H), 5.95 (dd, J=3.29, 1.75 Hz, 1H), 3.92-4.01 (m, 2H), 3.84-3.92 (m, 2H), 2.06 (s, 3H).

### Example 36:6-(6-Methylpyridin-2-yl)-5-(3-(methylsulfinyl)phenyl)-2,3-dihydro-1H-imidazo[1,2-a]imidazole

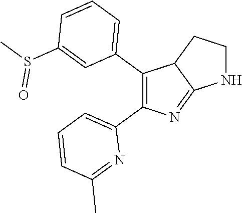 [SMILES: Cc1cccc(C2=C(c3cccc(S(C)=O)c3)C3CCNC3=N2)n1]

Prepared following procedure D, starting from Intermediate 59 (30 mg, 0.079 mmol). Purification by RP flash chromatography (gradient of elution from 0% to 30% of B in A; A: water/MeCN 95:5+0.1% HCOOH, B: MeCN/water 95:5+0.1% HCOOH) yielded the title compound (4 mg, 0.012 mmol, 14.99% yield) as yellow pale solid.

LC-MS (ESI): m/z (M+1) 339.3, rt=0.37 min (Method E).

¹H NMR (400 MHZ, DMSO-d6) δ ppm 7.97 (s, 1H), 7.68-7.75 (m, 1H), 7.55 (t, J=7.02 Hz, 1H), 7.50-7.53 (m, 3H), 6.94 (dd, J=7.02, 1.10 Hz, 1H), 6.18-6.25 (m, 1H), 3.98-4.10 (m, 2H), 3.77-3.87 (m, 2H), 2.72 (s, 3H), 2.24 (s, 3H).

### Example 37: 1-6-(6-Methylpyridin-2-yl)-5-(3-(methylsulfonyl)phenyl)-2,3-dihydro-1H-imidazo[1,2-a]imidazole

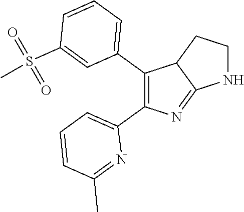 [SMILES: Cc1cccc(C2=C(c3cccc(S(C)(=O)=O)c3)C3CCNC3=N2)n1]

Prepared following procedure D, starting from Intermediate 61 (50 mg, 0.126 mmol). Purification by RP flash chromatography (gradient of elution from 0% to 30% of B in A; A: water/MeCN 95:5+0.1% HCOOH, B: MeCN/water 95:5+0.1% HCOOH) yielded the title compound (10 mg, 0.028 mmol, 22% yield) as pale yellow solid.

LC-MS (ESI): m/z (M+1) 355.4, rt=0.42 min (Method E).

¹H NMR (400 MHZ, DMSO-d6) δ ppm 8.27 (s, 1H), 7.88 (d, J=7.89 Hz, 1H), 7.79 (br d, J=7.89 Hz, 1H), 7.48-7.65 (m, 3H), 6.98 (d, J=7.45 Hz, 1H), 6.40 (br s, 1H), 3.99-4.14 (m, 2H), 3.79-3.92 (m, 2H), 3.18 (s, 3H), 2.26 (s, 3H).

### Example 38: 6-(6-Methylpyridin-2-yl)-5-(3-(methylthio)phenyl)-2,3-dihydro-1H-imidazo[1,2-a]imidazole

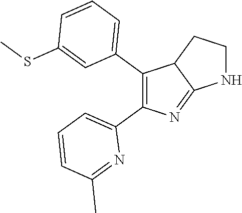 [SMILES: CSc1cccc(C2=C(c3cccc(C)n3)N=C3NCCC32)c1]

Prepared following procedure D, starting from Intermediate 57 (30 mg, 0.082 mmol). Purification by RP flash chromatography (gradient of elution from 0% to 30% of B in A; A: water/MeCN 95:5+0.1% HCOOH, B: MeCN/water 95:5+0.1% HCOOH) yielded the title compound (5 mg, 0.016 mmol, 19% yield) as pale yellow solid.

LC-MS (ESI): m/z (M+1) 323.2, rt=0.65 min (Method E).

¹H NMR (400 MHZ, DMSO-d6) δ ppm 7.62 (t, J=7.78 Hz, 1H), 7.45 (s, 1H), 7.37-7.43 (m, 1H), 7.25-7.37 (m, 2H), 7.16 (d, J=7.67 Hz, 1H), 7.10 (br d, J=7.89 Hz, 1H), 6.47 (s, 1H), 4.10-4.24 (m, 2H), 3.99-4.10 (m, 2H), 2.39-2.44 (m, 3H).

### Example 39: 3-(6-(6-Methylpyridin-2-yl)-2,3-dihydro-1H-imidazo[1,2-a]imidazol-5-yl)benzenesulfonamide

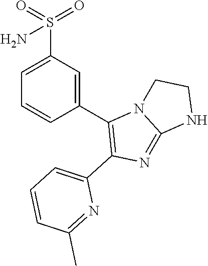 [SMILES: Cc1cccc(-c2nc3n(c2-c2cccc(S(N)(=O)=O)c2)CCN3)n1]

Prepared following procedure D, starting from Intermediate 54 (63 mg, 0.159 mmol). Purification by SCX (2-g cartridge, elution with 2N NH₃ in MeOH) yielded the title compound (37.9 mg, 0.107 mmol, 88% yield) as yellow solid.

LC-MS (ESI): m/z (M+1) 356.3, rt=0.38 min (Method E).

¹H NMR (400 MHZ, DMSO-d6) δ ppm 8.17 (t, J=1.53 Hz, 1H), 7.79 (d, J=7.67 Hz, 1H), 7.70 (d, J=7.89 Hz, 1H), 7.50-7.62 (m, 3H), 7.31 (s, 2H), 6.97 (dd, J=6.91, 1.43 Hz, 1H), 6.24-6.30 (m, 1H), 4.01-4.10 (m, 2H), 3.82-3.91 (m, 2H), 2.27 (s, 3H).

### Example 40: Methyl 2-(6-(6-methylpyridin-2-yl)-2,3-dihydro-1H-imidazo[1,2-a]imidazol-5-yl)-5-(methylthio)benzoate

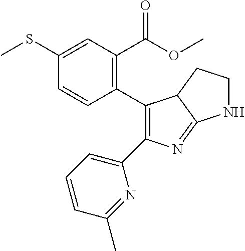 [SMILES: COC(=O)c1cc(SC)ccc1C1=C(c2cccc(C)n2)N=C2NCCC21]

Prepared followed procedure D, starting from Intermediate 63 (80 mg, 0.189 mmol). Purification by RP flash chromatography (gradient of elution from 0% to 30% of B in A; A: water/MeCN 95:5+0.1% HCOOH, B: MeCN/water 95:5+0.1% HCOOH) yielded the title compound (10 mg, 0.026 mmol, 14% yield) as pale yellow solid.

LC-MS (ESI): m/z (M+1) 381.4, rt=0.69 min (Method E).

¹H NMR (400 MHZ, DMSO-d6) δ ppm 7.65 (d, J=1.32 Hz, 1H), 7.38-7.52 (m, 4H), 6.84 (d, J=6.65 Hz, 1H), 6.12 (br s, 1H), 3.98-4.13 (m, 1H), 3.83 (br d, J=6.58 Hz, 2H), 3.69 (q, J=8.18 Hz, 1H), 3.37 (s, 3H), 2.12 (s, 3H).

### Example 41: Methyl 2-methoxy-5-(6-(6-methylpyridin-2-yl)-2,3-dihydro-1H-imidazo[1,2-a]imidazol-5-yl)benzoate

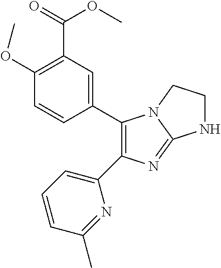 [SMILES: COC(=O)c1cc(-c2c(-c3cccc(C)n3)nc3n2CCN3)ccc1OC]

Prepared followed procedure D, starting from Intermediate 55 (80 mg, 0.197 mmol). Purification by NH-silica flash chromatography (gradient of elution from 0 to 100% of DCM/MeOH 9:1 in DCM) yielded the title compound (20 mg, 0.055 mmol, 27.9% yield) as a pale yellow solid.

LC-MS (ESI): m/z (M+1) 365.3, rt=0.83 min (Method E).

¹H NMR (400 MHZ, DMSO-d6) δ ppm 8.11 (d, J=2.20 Hz, 1H), 7.67 (dd, J=8.71, 2.32 Hz, 1H), 7.48-7.56 (m, 2H), 7.09 (d, J=8.82 Hz, 1H), 6.91 (d, J=7.06 Hz, 1H), 6.12 (br s, 1H), 3.95-4.03 (m, 2H), 3.77-3.84 (m, 5H), 3.74 (s, 3H), 2.22 (s, 3H).

### Example 42:2-(1-Ethyl-5-{thieno[3,2-c]pyridin-2-yl}-1H,2H,3H-imidazo[1,2-a] [1,3]diazol-6-yl)-6-methylpyridine

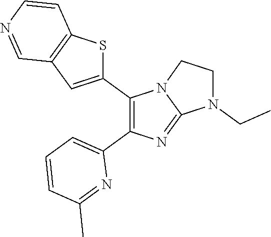 [SMILES: CCN1CCn2c1nc(-c1cccc(C)n1)c2-c1cc2cnccc2s1]

Prepared from Intermediate 43 (20 mg, 0.088 mmol) and Intermediate 8 (24 mg, 0.114 mmol) following procedure A. Purification by pTLC (SiO₂; Hexanes/EtOAc/MeOH/TEA 9:10:1:0.) yielded the title compound (19 mg, 0.053 mmol, 60% yield) as yellow powder.

LC-MS (ESI): m/z (M+1) 362.1, rt=1.70 min (Method D).

¹H NMR (400 MHZ, DMSO-d6) δ 9.04 (d, J=1.0 Hz, 1H), 8.35 (d, J=5.5 Hz, 1H), 8.00-7.92 (m, 2H), 7.74-7.60 (m, 2H), 7.15-7.08 (m, 1H), 4.34 (dd, J=8.5, 7.0 Hz, 2H), 3.85 (dd, J=8.8, 6.8 Hz, 2H), 3.28 (q, J=7.2 Hz, 2H), 2.54 (s, 3H), 1.23 (t, J=7.2 Hz, 3H).

### Example 43:2-Fluoro-5-(6-(6-methylpyridin-2-yl)-2,3-dihydro-1H-imidazo[1,2-a]imidazol-5-yl) aniline

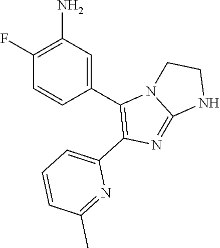 [SMILES: Cc1cccc(-c2nc3n(c2-c2ccc(F)c(N)c2)CCN3)n1]

Prepared following procedure D, starting from Intermediate 56 (27 mg, 0.077 mmol). Purification by SCX (2 g cartridge, elution with 2N NH₃ in MeOH) followed by NH-silica flash chromatography (gradient of elution from 0 to 100% of DCM/MeOH 9:1 in DCM) yielded the title compound (12.4 mg, 0.040 mmol, 52% yield) as a light-brown solid.

LC-MS (ESI): m/z (M+1) 310.1, rt=0.46 min (Method E).

¹H NMR (400 MHZ, DMSO-d6) δ ppm 7.45-7.54 (m, 1H), 7.40 (d, J=8.10 Hz, 1H), 7.02 (dd, J=8.99, 2.19 Hz, 1H), 6.86-6.93 (m, 2H), 6.67 (ddd, J=8.28, 4.55, 2.08 Hz, 1H), 6.01-6.07 (m, 1H), 4.99 (s, 2H), 3.89-3.99 (m, 2H), 3.75-3.83 (m, 2H), 2.26 (s, 3H).

### Example 44:5-Fluoro-2-(6-(6-methylpyridin-2-yl)-2,3-dihydro-1H-imidazo[1,2-a]imidazol-5-yl) aniline

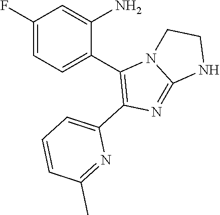 [SMILES: Cc1cccc(-c2nc3n(c2-c2ccc(F)cc2N)CCN3)n1]

Prepared following procedure D, starting from Intermediate 65 (100 mg, 0.285 mmol). Purification by SCX (5 g cartridge, elution with 7N NH₃ in MeOH) yielded the title compound (82.3 mg, 0.266 mmol, 93% yield) as yellow powder.

LC-MS (ESI): m/z (M+1) 310.3, rt=0.54 min (Method E).

¹H NMR (400 MHZ, DMSO-d6) δ ppm 7.46-7.53 (m, 1H), 7.41-7.46 (m, 1H), 7.06 (dd, J=8.44, 6.91 Hz, 1H), 6.85 (d, J=7.23 Hz, 1H), 6.49 (dd, J=11.73, 2.52 Hz, 1H), 6.31 (td, J=8.55, 2.63 Hz, 1H), 6.01 (s, 1H), 5.14 (s, 2H), 3.77 (br s, 4H), 2.19 (s, 3H).

### Example 45:2-Methoxy-5-(6-(6-methylpyridin-2-yl)-2,3-dihydro-1H-imidazo[1,2-a]imidazol-5-yl)benzoic acid

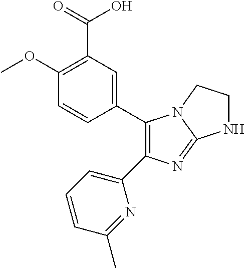 [SMILES: COc1ccc(-c2c(-c3cccc(C)n3)nc3n2CCN3)cc1C(=O)O]

Prepared by stirring a solution of Intermediate 55 (60 mg, 0.148 mmol) and 1M NaOH (0.738 mL, 1.476 mmol) in MeOH (1 mL) at reflux for 3 h. The reaction was concentrated under reduced pressure and the crude was purified by RP flash chromatography (gradient of elution from 0% to 60% of B in A; A: water/MeCN 95:5+0.1% HCOOH, B: MeCN/water 95:5+0.1% HCOOH) to give the title compound (12.4 mg, 0.035 mmol, 24% yield).

LC-MS (ESI): m/z (M+1) 351.2, rt=0.50 min (Method E).

¹H NMR (400 MHZ, DMSO-d6) δ ppm 8.06 (d, J=2.19 Hz, 1H), 7.67 (dd, J=8.66, 2.30 Hz, 1H), 7.49-7.58 (m, 2H), 7.08 (d, J=8.77 Hz, 1H), 6.93 (d, J=7.02 Hz, 1H), 6.13 (br s, 1H), 3.98-4.07 (m, 2H), 3.81-3.88 (m, 5H). 2.26 (s, 3H).

### Example 46:2-Methoxy-5-(6-(6-methylpyridin-2-yl)-2,3-dihydro-1H-imidazo[1,2-a]imidazol-5-yl) aniline

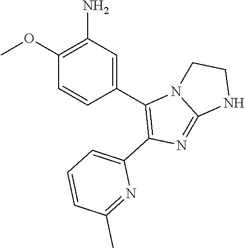 [SMILES: COc1ccc(-c2c(-c3cccc(C)n3)nc3n2CCN3)cc1N]

Prepared following procedure D, starting from Intermediate 69 (40 mg, 0.110 mmol). Purification by RP flash chromatography (gradient of elution from 0% to 40% of B in A; A: water/MeCN 95:5+0.1% HCOOH, B: MeCN/water 95:5+0.1% HCOOH). Proper fractions were collected and purified by SCX (2 g cartridge, elution with 7N NH₃ in MeOH). Final purification by NH-silica flash chromatography (gradient of elution from 0 to 100% of DCM/MeOH 9:1 in DCM) yielded the title compound (11 mg, 0.034 mmol, 31% yield).

LC-MS (ESI): m/z (M+1) 322.1, rt=0.44 min (Method E).

¹H NMR (400 MHZ, DMSO-d6) δ ppm 7.43-7.50 (m, 1H), 7.36 (d, J=7.89 Hz, 1H), 6.90 (d, J=1.10 Hz, 1H), 6.87 (d, J=7.45 Hz, 1H), 6.69-6.75 (m, 2H), 5.95-6.00 (m, 1H), 4.58 (s, 2H), 3.88-3.97 (m, 2H), 3.76-3.81 (m, 2H), 3.74 (s, 3H), 2.27 (s, 3H).

### Example 47:5-(1-Ethyl-6-(6-methylpyridin-2-yl)-2,3-dihydro-1H-imidazo[1,2-a]imidazol-5-yl)-2-fluoroaniline

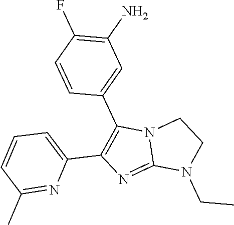 [SMILES: CCN1CCn2c1nc(-c1cccc(C)n1)c2-c1ccc(F)c(N)c1]

To a suspension of Intermediate 56 (100 mg, 0.285 mmol) and tris(triphenylphosphine)rhodium(I) carbonyl hydride (26.1 mg, 0.028 mmol) in THF (1 mL) under nitrogen atmosphere, diphenylsilane (0.132 mL, 0.711 mmol) was added and the mixture was stirred at 70° C. for 1 h. The reaction was quenched with 2N aq. HCl, then the mixture was partitioned between EtOAC and a sat. aq. solution of NaHCO₃. The organic phase was dried over Na₂SO₄, filtered through a phase separator tube and concentrated under reduced pressure. Purification by NH-silica flash chromatography (gradient of elution from 0 to 100% of DCM/EtOH 9:1 in DCM), followed by purification by SCX (2 g cartridge, elution with 7N NH₃ in MeOH) yielded the title compound (2.7 mg, 8.00 μmol, 3% yield).

LC-MS (ESI): m/z (M+1) 338.4, rt=0.53 min (Method E).

¹H NMR (400 MHZ, DMSO-d6) δ ppm 7.51 (t, J=7.71 Hz, 1H), 7.39 (br d, J=7.67 Hz, 1H), 7.00 (dd, J=8.88, 1.86 Hz, 1H), 6.87-6.97 (m, 2H), 6.64-6.70 (m, 1H), 5.04 (br s, 2H), 3.89-4.00 (m, 2H), 3.66-3.74 (m, 2H), 3.20 (q, J=7.16 Hz, 2H), 2.29 (s, 3H), 1.18 (t, J=7.23 Hz, 3H).

### Example 48:6-(6-(6-Methylpyridin-2-yl)-2,3-dihydro-1H-imidazo[1,2-a]imidazol-5-yl)benzo[d] thiazol-2-amine

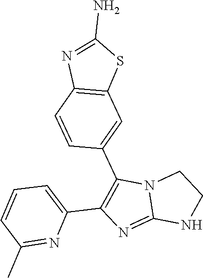 [SMILES: Cc1cccc(-c2nc3n(c2-c2ccc4nc(N)sc4c2)CCN3)n1]

Prepared from Intermediate 20 (102 mg, 0.319 mmol) and Intermediate 71 (180 mg, 0.478 mmol) following procedure B. Purification by NH-silica flash chromatography (gradient of elution from 0 to 100% of DCM/MeOH 9:1 in DCM), followed by purification by silica flash chromatography (gradient of elution from 0 to 100% of MeOH in DCM) yielded the title compound (4 mg, 0.011 mmol, 3.60% yield).

LC-MS (ESI): m/z (M+1) 349.3, rt=0.38 min (Method E).

¹H NMR (400 MHZ, DMSO-d6) δ ppm 7.98 (s, 1H) 7.40-7.54 (m, 5H) 7.38 (dd, J=8.38, 1.54 Hz, 1H) 7.24 (d, J=8.46 Hz, 1H) 6.89 (d, J=7.10 Hz, 1H) 6.03-6.10 (m, 1H) 3.95-4.05 (m, 2H) 3.74-3.84 (m, 2H) 2.22 (s, 3H).

### Example 49:2-(6-(6-Methylpyridin-2-yl)-2,3-dihydro-1H-imidazo[1,2-a]imidazol-5-yl)-5-(methylthio) aniline

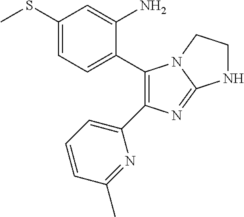 [SMILES: CSc1ccc(-c2c(-c3cccc(C)n3)nc3n2CCN3)c(N)c1]

A mixture of sodium methanethiolate (77 mg, 1.096 mmol) and Intermediate 65 (128.4 mg, 0.365 mmol) in DMF (2 mL) was stirred at 100° C. for 72 h. Purification by RP flash chromatography (gradient of elution from 0% to 20% of B in A; A: water/MeCN 95:5+0.1% HCOOH, B: MeCN/water 95:5+0.1% HCOOH) followed by concentration of fractions, neutralization of formate salt and NH-silica flash chromatography (gradient elution from 0 to 20% A in dichloromethane, A: dichloromethane/MeOH 9:1) yielded the title compound (17.9 mg, 0.053 mmol, 14.5% yield).

LC-MS (ESI): m/z (M+1) 338.3, rt=0.60 min (Method E).

¹H NMR (400 MHZ, DMSO-d6) δ ppm 7.50-7.56 (m, 1H) 7.46 (d, J=7.51 Hz, 1H) 7.04 (d, J=7.89 Hz, 1H) 6.89 (d, J=7.23 Hz, 1H) 6.68 (d, J=1.75 Hz, 1H) 6.47 (dd, J=8.11, 1.97 Hz, 1H) 6.04 (s, 1H) 5.01 (s, 2H) 3.80 (br s, 4H) 2.43 (s, 3H) 2.25 (s, 3H).

### Example 50: Methyl 5-(6-(6-methylpyridin-2-yl)-2,3-dihydro-1H-imidazo[1,2-a]imidazol-5-yl)-2-(methylthio)benzoate

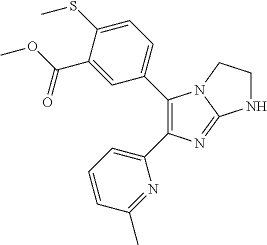 [SMILES: COC(=O)c1cc(-c2c(-c3cccc(C)n3)nc3n2CCN3)ccc1SC]

Prepared according to procedure D, starting from Intermediate 72 (45 mg, 0.107 mmol). Purification by RP flash chromatography (gradient of elution from 0% to 40% of B in A; A: water/MeCN 95:5+0.1% HCOOH, B: MeCN/water 95:5+0.1% HCOOH) followed concentration of fractions and NH-silica flash chromatography (gradient elution from 0 to 40% A in heptane, A: dichloromethane/MeOH 9:1) yielded the title compound (15.0 mg, 0.039 mmol, 37% yield).

LC-MS (ESI): m/z (M+1) 381, rt=0.71 min (Method E).

¹H NMR (400 MHZ, DMSO-d6) δ ppm 8.42 (d, J=1.97 Hz, 1H), 7.71 (dd, J=8.44, 2.08 Hz, 1H), 7.48-7.58 (m, 2H), 7.32 (d, J=8.55 Hz, 1H), 6.93 (dd, J=6.69, 1.64 Hz, 1H), 6.19 (s, 1H), 3.96-4.09 (m, 2H), 3.78-3.84 (m, 5H), 2.42 (s, 3H), 2.23 (s, 3H).

### Example 51:5-Methoxy-2-(6-(6-methylpyridin-2-yl)-2,3-dihydro-1H-imidazo[1,2-a]imidazol-5-yl) aniline

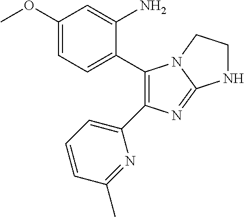 [SMILES: COc1ccc(-c2c(-c3cccc(C)n3)nc3n2CCN3)c(N)c1]

Prepared according to procedure D, starting from Intermediate 74 (73.2 mg, 0.183 mmol). The reaction mixture was filtered through an SCX column. Eluted fractions were concentrated under reduced pressure. Purification by NH-silica flash chromatography (gradient elution from 0 to 20% A in DCM; A: DCM/MeOH 9:1) yielded the title compound (24.1 mg, 0.075 mmol, 41% yield).

LC-MS (ESI): m/z (M+1) 322.3, rt=0.51 min (Method E).

¹H NMR (400 MHZ, DMSO-d6) δ ppm 7.48 (t, J=7.78 Hz, 1H) 7.39 (d, J=7.89 Hz, 1H) 6.96 (d, J=8.33 Hz, 1H) 6.84 (d, J=7.45 Hz, 1H) 6.32 (d, J=2.41 Hz, 1H) 6.15 (dd, J=8.44, 2.52 Hz, 1H) 5.96 (s, 1H) 4.91 (br s, 2H) 3.76 (br s, 4H) 3.67 (s, 3H) 2.23 (s, 3H).

### Example 52:2-(6-(6-Methylpyridin-2-yl)-2,3-dihydro-1H-imidazo[1,2-a]imidazol-5-yl)-5-(methylthio)benzoic acid

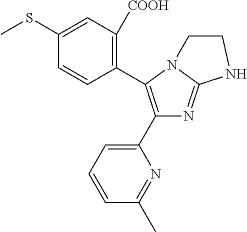 [SMILES: CSc1ccc(-c2c(-c3cccc(C)n3)nc3n2CCN3)c(C(=O)O)c1]

To a solution of Example 40 (10 mg, 0.026 mmol) in EtOH (3 mL), aqueous NaOH 2M (2 mL, 4.00 mmol) was added and the solution was stirred for 12 h. Volatiles were removed under reduced pressure. Purification by RP flash chromatography (gradient of elution from 0% to 50% of B in A; A: water/MeCN 95:5+0.1% HCOOH, B: MeCN/water 95:5+0.1% HCOOH) yielded the title compound (2 mg, 5.46 μmol, 21% yield).

LC-MS (ESI): m/z (M+1) 367.3, rt=0.62 min (Method E).

### Example 53:6-(6-(6-Methylpyridin-2-yl)-2,3-dihydro-1H-imidazo[1,2-a]imidazol-5-yl)benzo[d] thiazol-2-ol

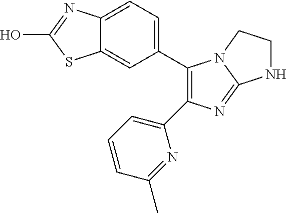 [SMILES: Cc1cccc(-c2nc3n(c2-c2ccc4nc(O)sc4c2)CCN3)n1]

To a solution of Intermediate 77 (30 mg, 0.074 mmol) in dry DCM (2 mL), BBr3 (0.740 mL, 0.740 mmol) was added and the reaction was stirred at RT for 12 h. Then, the reaction was cooled to 0° C. and EtOH (10 mL) was added. The resulting clear solution was concentrated to half volume and it was heated to 60° C. for 8 h. Volatiles were removed under reduced pressure. Purification by RP flash chromatography (gradient of elution from 0% to 70% of B in A; A: water/MeCN 95:5+0.1% HCOOH, B: MeCN/water 95:5+0.1% HCOOH) yielded the title compound (10 mg, 0.029 mmol, 39% yield).

LC-MS (ESI): m/z (M+1) 350.3, rt=0.48 min (Method E).

¹H-NMR (400 MHZ, DMSO-d6) δ ppm 8.27 (s, 1H) 7.89 (d, J=1.32 Hz, 1H) 7.50-7.55 (m, 1H) 7.45-7.50 (m, 2H) 7.05 (d, J=8.33 Hz, 1H) 6.92 (d, J=7.02 Hz, 1H) 6.13 (br s, 1H) 3.91-4.11 (m, 2H) 3.77-3.84 (m, 2H) 2.23 (s, 3H).

### Example 54:5-(6-(6-Methylpyridin-2-yl)-2,3-dihydro-1H-imidazo[1,2-a]imidazol-5-yl)-2-(methylthio) aniline

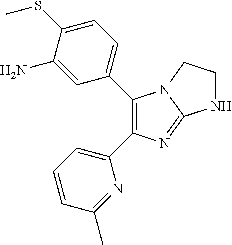 [SMILES: CSc1ccc(-c2c(-c3cccc(C)n3)nc3n2CCN3)cc1N]

Prepared according to procedure D, starting from Intermediate 80 (8.0 mg, 0.021 mmol). Purification by SCX (1 g cartridge, elution with 7N NH₃ in MeOH) yielded the title compound (5.0 mg, 0.015 mmol, 70% yield).

LC-MS (ESI): m/z (M+1) 338.3, rt=0.59 min (Method E).

¹H NMR (400 MHZ, DMSO-d6) δ ppm 7.55 (t, J=7.72 Hz, 1H), 7.40 (br d, J=7.72 Hz, 1H), 7.15 (d, J=8.16 Hz, 1H), 6.99 (d, J=1.10 Hz, 1H), 6.97 (br d, J=7.50 Hz, 1H), 6.78 (dd, J=8.05, 1.43 Hz, 1H), 5.04-5.22 (m, 2H), 3.94-4.09 (m, 2H), 3.78-3.93 (m, 2H), 2.34 (br s, 3H), 2.33 (br s, 3H).

### Example 55:4-[6-(6-Methylpyridin-2-yl)-1H,2H,3H-imidazo[1,2-a][1,3]diazol-5-yl]phenol

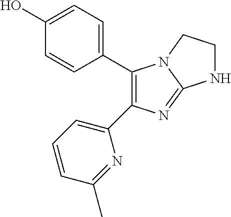 [SMILES: Cc1cccc(-c2nc3n(c2-c2ccc(O)cc2)CCN3)n1]

Prepared following the procedure for the synthesis of Example 53, starting from Intermediate 81 (120 mg, 0.344 mmol). Reaction mixture was dried under reduced pressure, suspended in MeOH (2 mL) and alkalized to pH=12 using 1M aq. NaOH solution. The solution was stirred for 3 h at 90° C. The mixture was dried under reduced pressure and the residue was dissolved in water and neutralized using 1M aq. HCl and sat. aq. NaHCO₃ solution. The resulting suspension was extracted with DCM. The combined organic layers were dried over Na₂SO₄, filtered and dried under reduced pressure. The residue was triturated with small amount of MeOH yielding the title compound (12.5 mg, 0.042 mmol, 13% yield).

LC-MS (ESI): m/z (M+1) 293.32, rt=2.913 min (Method C).

¹H NMR (400 MHZ, DMSO-d6) δ 9.46 (s, 1H), 7.52 (t, J=7.7 Hz, 1H), 7.44 (dd, J=8.2, 6.2 Hz, 3H), 6.91 (d, J=7.5 Hz, 1H), 6.73 (d, J=8.2 Hz, 2H), 6.06 (d, J=2.9 Hz, 1H), 3.98 (t, J=7.7 Hz, 2H), 3.82 (d, J=9.0 Hz, 2H), 2.27 (s, 3H).

### Example 56:4-[6-(6-Methylpyridin-2-yl)-1H,2H,3H-imidazo[1,2-a][1,3]diazol-5-yl]pyridin-2-amine

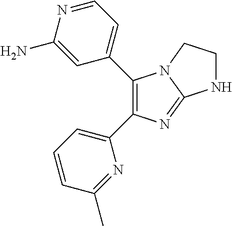 [SMILES: Cc1cccc(-c2nc3n(c2-c2ccnc(N)c2)CCN3)n1]

Prepared according to procedure D, starting from Intermediate 82 (60.0 mg, 0.180 mmol). Volatiles were removed under reduced pressure. Purification by flash chromatography eluting with DCM:MeOH:water (90:10:0.5) yielded the title compound (45 mg, 0.154 mmol, 86% yield).

LC-MS (ESI): m/z (M+1): 293.3 (Method F).

¹H NMR (400 MHZ, DMSO-d6) δ ppm 7.82 (d, J=5.5 Hz, 1H), 7.55-7.63 (m, 1H), 7.48 (d, J=7.9 Hz, 1H), 7.00 (d, J=7.2 Hz, 1H), 6.71 (d, J=0.7 Hz, 1H), 6.64 (dd, J=5.5, 1.5 Hz, 1H), 6.24 (t, J=2.4 Hz, 1H), 5.76 (s, 2H), 4.01-4.13 (m, 2H), 3.80-3.91 (m, 2H), 2.33 (s, 3H).

### Example 57:6-[6-(6-Methoxypyridin-2-yl)-1H,2H,3H-imidazo[1,2-a][1,3]diazol-5-yl]-1,3-benzothiazole

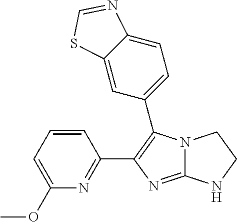 [SMILES: COc1cccc(-c2nc3n(c2-c2ccc4ncsc4c2)CCN3)n1]

Prepared according to procedure D, starting from Intermediate 86 (223.0 mg, 0.352 mmol). Purification by RP flash chromatography (gradient of elution from 0% to 30% of B in A; A: water/MeCN 95:5+0.1% HCOOH, B: MeCN/water 95:5+0.1% HCOOH) yielded the title compound (60 mg, 0.172 mmol, 49% yield).

LC-MS (ESI): m/z (M+1): 350.2 (Method E).

¹H NMR (500 MHZ, DMSO-d6) δ ppm 9.37 (s, 1H), 8.39 (d, J=1.2 Hz, 1H), 8.06 (d, J=8.4 Hz, 1H), 7.70 (dd, J=8.4, 1.7 Hz, 1H), 7.57-7.64 (m, 1H), 7.41 (dd, J=7.5, 0.8 Hz, 1H), 6.49 (dd, J=8.1, 0.8 Hz, 1H), 6.24 (s, 1H), 3.97-4.13 (m, 2H), 3.85 (s, 2H), 3.22 (s, 3H).

### Example 58:6-[5-(1,3-Benzothiazol-6-yl)-1H,2H,3H-imidazo[1,2-a][1,3]diazol-6-yl]pyridin-2-ol

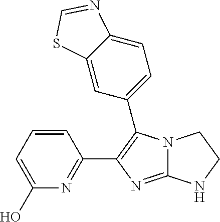 [SMILES: Oc1cccc(-c2nc3n(c2-c2ccc4ncsc4c2)CCN3)n1]

To a solution of Example 57 (16.0 mg, 0.050 mmol) in 1,4-Dioxane (0.4 mL), aq. 4N HCl (0.4 mL, 1.6 mmol) was added and the mixture was stirred at 85° C. for 16 h. Volatiles were removed under reduced pressure. Purification by RP flash chromatography (gradient of elution from 0% to 35% of B in A; A: water/MeCN 95:5+0.1% NH₄OH, B: MeCN/water 95:5+0.1% NH₄OH) yielded the title compound (4.5 mg, 0.013 mmol, 29% yield).

LC-MS (ESI): m/z (M+1): 336.1 (Method E).

¹H NMR (400 MHZ, DMSO-d6) δ ppm 10.21 (br s, 1H), 9.45 (s, 1H), 8.34 (d, J=1.5 Hz, 1H), 8.15 (d, J=8.6 Hz, 1H), 7.62 (dd, J=8.4, 1.6 Hz, 1H), 7.20-7.29 (m, 1H), 6.49 (br s, 1H), 5.96-6.18 (m, 2H), 4.02-4.14 (m, 2H), 3.83-3.93 (m, 2H).

### Pharmacological Activity of the Compounds of the Invention

### In Vitro Assay

The enzymatic activity of compounds of the present invention was monitored measuring the formation of ADP using the ADP-GLO Kinases assay. Following the incubation of the purified enzyme, a substrate and ATP, the produced ADP was converted into ATP, which in turn was converted into light by Ultra-Glo Luciferase. The luminescent signal positively correlated with ADP amount and kinase activity. Briefly, the kinase reaction was performed by incubating 2.6 nM of the purified, commercially available human ALK5 (recombinant TGF β1 N-term GST-tagged, 80-end), a final concentration of TGFβ1 peptide 94.5 μM (Promega, T36-58) and ultra-pure ATP (Promega V915B). The ATP concentration was set at the Km value (concentration of substrate which permits the enzyme to achieve half maximal velocity (Vmax)) of ALK5 (5 μM). All reactions/incubations were performed at 25° C. Compound and ALK5 kinase were mixed and incubated for 15 mins. Reactions were initiated by addition of ATP at a final concentration in the assay of 0.83 μM. After an incubation of 150 min, the reaction was stopped, and ADP production detected with ADP-Glo kit according to manufacturer's indications. The assay was performed in 384-well format and was validated using a selection of reference compounds that was tested in 11 point concentration-response curve.

The results for individual compounds are provided below in Table 8 wherein the compounds are classified in term of potency (nM) with respect to their inhibitory activity on ALK5 receptor.

TABLE 8

| Example No | ALK5 IC50 (nM) |
|---|---|
| 1-4, 7, 12-14, 17-19, 22, 31, 33, 57 | <1 |
| 9, 20, 21, 26, 29, 30, 35, 42, 43, 48, 55 | 1-5 |
| 8, 15, 56 | 5-10 |

As it can be appreciated, all the compounds of Table 8 show a good activity as antagonists of ALK5 receptor.

1. A compound of formula (I) 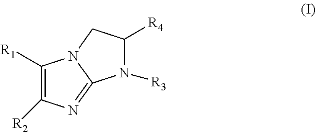
wherein
R₁ is selected from the group consisting of aryl optionally substituted by one or more groups selected from halogen atoms, NH₂, —OH, —O(C₁-C₆)alkyl, —S—(C₁-C₆)alkyl, —S(O)(C₁-C₆)alkyl, —S(O)₂—(C₁-C₆)alkyl, —S(O)₂—NH₂, —C(O)OH and —C(O)O—(C₁-C₆)alkyl; heterocycloalkyl optionally substituted by a oxo group; pyridyl substituted by NH₂; phenyl, pyridyl or thienyl fused with a structural moiety, which together with two ring members of said phenyl, pyridyl or thienyl, forms a 5-7 membered aromatic or non-aromatic ring, wherein said ring optionally contains up to three heteroatoms selected from N, O and S;
R₂ is aryl optionally substituted by one or more groups selected from halogen atoms and —(C₁-C₆)alkyl; or R₂ is heteroaryl optionally substituted by one or more groups selected from —(C₁-C₆)alkyl, —O—(C₁-C₆)alkyl, —OH and halogen atoms;
R₃ is H or is independently selected from the group consisting of —(C₁-C₆)alkyl and —C(O)—(C₁-C₆)alkyl;
R₄ is H or is selected from the group consisting of —(C₁-C₆)hydroxyalkyl, —C(O)OR₅, —C(O)O—(C₁-C₆)alkyl, —C(O)—NR₅R₆, —(C₁-C₆)alkylene-C(O)OH and —(C₁-C₆)alkylene-C(O)O—(C₁-C₆)alkyl;
R₅ is H or —(C₁-C₆)alkyl; and
R₆ is —(C₁-C₆)alkyl;
or a pharmaceutically acceptable salt thereof.

2. The compound or pharmaceutically acceptable salt according to claim 1, wherein
R₁ is aryl optionally substituted by one or more groups selected from halogen atoms, —NH₂, —OH, —O(C₁-C₆)alkyl, —S—(C₁-C₆)alkyl, —S(O)(C₁-C₆) alkyl, —S(O)₂—(C₁-C₆) alkyl, —S(O)₂—NH₂, —C(O)OH and —C(O)O—(C₁-C₆)alkyl;
R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H or —(C₁-C₆)alkyl; and
R₄ is H.

3. The compound or pharmaceutically acceptable salt according to claim 2, selected from the group consisting of:
6-(1-Methyl-1H-pyrazol-3-yl)-5-(4-(methylthio)phenyl)-2,3-dihydro-1H-imidazo[1,2-a]imidazole;
6-(6-methylpyridin-2-yl)-5-(4-(methylsulfonyl)phenyl)-2,3-dihydro-1H-imidazo[1,2-a]imidazole;
6-(6-Methylpyridin-2-yl)-5-(4-(methylsulfinyl)phenyl)-2,3-dihydro-1H-imidazo[1,2-a]imidazole;
6-(6-Methylpyridin-2-yl)-5-(3-(methylsulfinyl)phenyl)-2,3-dihydro-1H-imidazo[1,2-a]imidazole;
1-6-(6-Methylpyridin-2-yl)-5-(3-(methylsulfonyl)phenyl)-2,3-dihydro-1H-imidazo[1,2-a]imidazole;
6-(6-Methylpyridin-2-yl)-5-(3-(methylthio)phenyl)-2,3-dihydro-1H-imidazo[1,2-a]imidazole;
3-(6-(6-Methylpyridin-2-yl)-2,3-dihydro-1H-imidazo[1,2-a]imidazol-5-yl)benzenesulfonamide;
Methyl 2-(6-(6-methylpyridin-2-yl)-2,3-dihydro-1H-imidazo[1,2-a]imidazol-5-yl)-5-(methylthio)benzoate;
Methyl 2-methoxy-5-(6-(6-methylpyridin-2-yl)-2,3-dihydro-1H-imidazo[1,2-a]imidazol-5-yl)benzoate;
2-Fluoro-5-(6-(6-methylpyridin-2-yl)-2,3-dihydro-1H-imidazo[1,2-a]imidazol-5-yl) aniline;
5-Fluoro-2-(6-(6-methylpyridin-2-yl)-2,3-dihydro-1H-imidazo[1,2-a]imidazol-5-yl) aniline;
2-Methoxy-5-(6-(6-methylpyridin-2-yl)-2,3-dihydro-1H-imidazo[1,2-a]imidazol-5-yl)benzoic acid;
2-Methoxy-5-(6-(6-methylpyridin-2-yl)-2,3-dihydro-1H-imidazo[1,2-a]imidazol-5-yl) aniline;
5-(1-Ethyl-6-(6-methylpyridin-2-yl)-2,3-dihydro-1H-imidazo[1,2-a]imidazol-5-yl)-2-fluoroaniline;
2-(6-(6-Methylpyridin-2-yl)-2,3-dihydro-1H-imidazo[1,2-a]imidazol-5-yl)-5-(methylthio) aniline;
Methyl 5-(6-(6-methylpyridin-2-yl)-2,3-dihydro-1H-imidazo[1,2-a]imidazol-5-yl)-2-(methylthio)benzoate;
5-Methoxy-2-(6-(6-methylpyridin-2-yl)-2,3-dihydro-1H-imidazo[1,2-a]imidazol-5-yl) aniline;
2-(6-(6-methylpyridin-2-yl)-2,3-dihydro-1H-imidazo[1,2-a]imidazol-5-yl)-5-(methylthio)benzoic acid;
5-(6-(6-Methylpyridin-2-yl)-2,3-dihydro-1H-imidazo[1,2-a]imidazol-5-yl)-2-(methylthio) aniline; and
4-[6-(6-Methylpyridin-2-yl)-1H,2H,3H-imidazo[1,2-a][1,3]diazol-5-yl]phenol.

4. The compound or pharmaceutically acceptable salt according to claim 1, wherein R₁ is group R₁ₓ 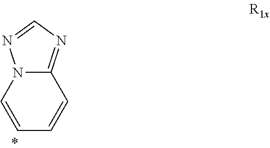
represented by the formula (Ix) 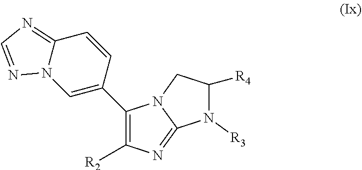
R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H or ethyl; and
R₄ is H.

5. The compound or pharmaceutically acceptable salt according to claim 4, selected from the group consisting of:
6-(6-(5-Chloro-2-fluorophenyl)-2,3-dihydro-1H-imidazo[1,2-a]imidazol-5-yl)-[1,2,4]triazolo[1,5-a]pyridine;
2-(5-([1,2,4]Triazolo[1,5-a]pyridin-6-yl)-2,3-dihydro-1H-imidazo[1,2-a]imidazol-6-yl)-4-methylthiazole;
6-(6-(6-Chloropyridin-2-yl)-2,3-dihydro-1H-imidazo[1,2-a]imidazol-5-yl)-[1,2,4]triazolo[1,5-a]pyridine; and
6-(1-Ethyl-6-(6-methylpyridin-2-yl)-2,3-dihydro-1H-imidazo[1,2-a]imidazol-5-yl)-[1,2,4]triazolo[1,5-a]pyridine.

6. The compound or pharmaceutically acceptable salt according to claim 1, wherein R₁ is group R_{1y} 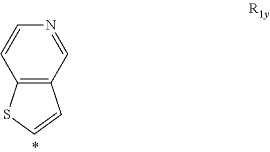
represented by the formula (Iy) 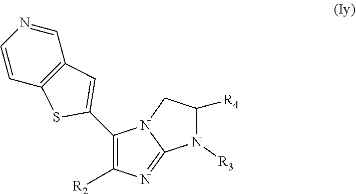
R₂ is aryl optionally substituted by one or more groups selected from halogen atoms and —(C₁-C₆)alkyl; or R₂ is heteroaryl optionally substituted by one or more groups selected from —(C₁-C₆)alkyl and halogen atoms;
R₃ is H; and
R₄ is H or is selected from the group consisting of hydroxymethyl, —C(O)OH, and N-methylacetamide.

7. The compound of formula (!) or pharmaceutically acceptable salt according to claim 6, selected from the group consisting of:
[6-(6-Methylpyridin-2-yl)-5-{thieno[3,2-c]pyridin-2-yl}-1H,2H,3H-imidazo[1,2-a][1,3]diazol-2-yl]methanol;
6-(6-Methylpyridin-2-yl)-5-{thieno[3,2-c]pyridin-2-yl}-1H,2H,3H-imidazo[1,2-a][1,3]diazole-2-carboxylic acid;
2-(6-(4-Methylthiazol-2-yl)-2,3-dihydro-1H-imidazo[1,2-a]imidazol-5-yl)thieno[3,2-c]pyridine;
2-(6-(6-Chloropyridin-2-yl)-2,3-dihydro-1H-imidazo[1,2-a]imidazol-5-yl)thieno[3,2-c]pyridine;
2-(6-(5-Chloro-2-fluorophenyl)-2,3-dihydro-1H-imidazo[1,2-a]imidazol-5-yl)thieno[3,2-c]pyridine; and
N-methyl-6-(6-methylpyridin-2-yl)-5-{thieno[3,2-b]pyridin-2-yl}-1H,2H,3H-imidazo[1,2-a][1,3]diazole-2-carboxamide.

8. A pharmaceutical composition comprising a compound of or pharmaceutically acceptable salt according to claim 1, in admixture with one or more pharmaceutically acceptable carriers, excipients, or both.

9. The pharmaceutical composition according to claim 8, formulated for administration by inhalation.

10. (canceled)

11. A method for treating a disease, disorder or condition mediated by ALK5 signaling pathway in a mammal, comprising administering the pharmaceutical composition of claim 8 to the mammal.

12. The method according to claim 11, wherein the disease, disorder or condition mediated by ALK5 signaling pathway comprises fibrosis and/or a disease, disorder or condition that involves fibrosis.

13. The method according to claim 12, wherein the fibrosis comprises at least one selected from the group consisting pulmonary fibrosis, idiopathic pulmonary fibrosis (IPF), hepatic fibrosis, renal fibrosis, ocular fibrosis, cardiac fibrosis, arterial fibrosis and systemic sclerosis.

14. The method according to claim 13, wherein the fibrosis comprises idiopathic pulmonary fibrosis (IPF).
